(19) United States
(12) Patent Application Publication
(10) Pub. No.: US 2020/0395099 A1
(43) Pub. Date: Dec. 17, 2020

(54) TECHNIQUES FOR PROTEIN IDENTIFICATION USING MACHINE LEARNING AND RELATED SYSTEMS AND METHODS

(71) Applicant: Quantum-Si Incorporated, Guilford, CT (US)

(72) Inventors: Michael Meyer, Guilford, CT (US); Brian Reed, Madison, CT (US); Zhizhuo Zhang, Guilford, CT (US); Sabrina Rashid, New Haven, CT (US); Bradley Robert Parry, Branford, CT (US)

(73) Assignee: Quantum-Si Incorporated, Guilford, CT (US)

(21) Appl. No.: 16/900,582

(22) Filed: Jun. 12, 2020

Related U.S. Application Data
(60) Provisional application No. 62/860,750, filed on Jun. 12, 2019.

(51) Int. Cl. G16B 40/30; G06N 3/04; G06N 3/08; G16B 5/00

(52) U.S. Cl. CPC G16B 40/30; G16B 5/00; G06N 3/088; G06N 3/0454

(57) ABSTRACT

Described herein are systems and techniques for identifying polypeptides using data collected by a protein sequencing device. The protein sequencing device may collect data obtained from detected light emissions by luminescent labels during binding interactions of reagents with amino acids of the polypeptide. The light emissions may result from application of excitation energy to the luminescent labels. The device may provide the data as input to a trained machine learning model to obtain output that may be used to identify the polypeptide. The output may indicate, for each of a plurality of locations in the polypeptide, one or more likelihoods that one or more respective amino acids is present at the location. The output may be matched to an amino acid sequence that specifies a protein.

### CROSS-REFERENCE TO RELATED APPLICATIONS

The present application claims the benefit under 35 U.S.C. § 119(e) of U.S. Provisional Patent Application No. 62/860,750, filed Jun. 12, 2019, titled “Machine Learning Enabled Protein Identification,” which is hereby incorporated by reference in its entirety.

### BACKGROUND

The present disclosure relates generally to identification of proteins. Proteomics has emerged as an important and necessary complement to genomics and transcriptomics in the study of biological systems. The proteomic analysis of an individual organism can provide insight into cellular processes and response patterns, which lead to improved diagnostic and therapeutic strategies. The complexity of protein structure, composition, and modification presents challenges in identification of proteins.

### SUMMARY

Described herein are systems and techniques for identifying proteins using data collected by a protein sequencing device. The protein sequencing device may collect data for binding interactions of reagents with amino acids of the protein. For example, the data may include data detected from light emissions resulting from application of excitation energy to the luminescent labels. The device may provide the data as input to a trained machine learning model to obtain output that may be used to identify a polypeptide. The output may indicate, for each of a plurality of locations in the polypeptide, one or more likelihoods that one or more respective amino acids is present at the location. The output may be matched to an amino acid sequence that specifies a protein.

According to some aspects, a method is provided for identifying a polypeptide, the method comprising using at least one computer hardware processor to perform accessing data for binding interactions of one or more reagents with amino acids of the polypeptide, providing the data as input to a trained machine learning model to obtain output indicating, for each of a plurality of locations in the polypeptide, one or more likelihoods that one or more respective amino acids is present at the location, and identifying the polypeptide based on the output obtained from the trained machine learning model.

According to some aspects, a system is provided for identifying a polypeptide, the system comprising at least one processor, and at least one non-transitory computer-readable storage medium storing instructions that, when executed by the at least one processor, cause the at least one processor to perform a method comprising accessing data for binding interactions of one or more reagents with amino acids of the polypeptide, providing the data as input to a trained machine learning model to obtain output indicating, for each of a plurality of locations in the polypeptide, one or more likelihoods that one or more respective amino acids is present at the location, and identifying the polypeptide based on the output obtained from the trained machine learning model.

According to some aspects, at least one non-transitory computer-readable storage medium is provided storing instructions that, when executed by at least one processor, cause the at least one processor to perform a method, the method comprising accessing data for binding interactions of one or more reagents with amino acids of a polypeptide, providing the data as input to a trained machine learning model to obtain output indicating, for each of a plurality of locations in the polypeptide, one or more likelihoods that one or more respective amino acids is present at the location, and identifying the polypeptide based on the output obtained from the trained machine learning model.

According to some aspects, a method is provided of training a machine learning model for identifying amino acids of polypeptides, the method comprising using at least one computer hardware processor to perform accessing training data obtained for binding interactions of one or more reagents with amino acids and training the machine learning model using the training data to obtain a trained machine learning model for identifying amino acids of polypeptides.

According to some aspects, a system is provided for training a machine learning model for identifying amino acids of polypeptides, the system comprising at least one processor, and at least one non-transitory computer-readable storage medium storing instructions that, when executed by the at least one processor, cause the at least one processor to perform accessing training data obtained for binding interactions of one or more reagents with amino acids, and training the machine learning model using the training data to obtain a trained machine learning model for identifying amino acids of polypeptides.

According to some aspects, at least one non-transitory computer-readable storage medium is provided storing instructions that, when executed by at least one processor, cause the at least one processor to perform accessing training data obtained for binding interactions of one or more reagents with amino acids, and training a machine learning model using the training data to obtain a trained machine learning model for identifying amino acids of polypeptides.

The foregoing apparatus and method embodiments may be implemented with any suitable combination of aspects, features, and acts described above or in further detail below. These and other aspects, embodiments, and features of the present teachings can be more fully understood from the following description in conjunction with the accompanying drawings.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear. For purposes of clarity, not every component may be labeled in every drawing.

FIG. 1A shows example configurations of labeled affinity reagents, including labeled enzymes and labeled aptamers which selectively bind with one or more types of amino acids, in accordance with some embodiments of the technology described herein;

FIG. 1B shows a degradation-based process of polypeptide sequencing using labeled affinity reagents, in accordance with some embodiments of the technology described herein;

FIG. 1C shows a process of polypeptide sequencing using a labeled polypeptide, in accordance with some embodiments of the technology described herein;

FIGS. 2A-2B illustrate polypeptide sequencing by detecting a series of signal pulses produced by light emission from association events between affinity reagents labeled with luminescent labels, in accordance with some embodiments of the technology described herein;

FIG. 2C depicts an example of polypeptide sequencing by iterative terminal amino acid detection and cleavage, in accordance with some embodiments of the technology described herein;

FIG. 2D shows an example of polypeptide sequencing in real-time using labeled exopeptidases that each selectively binds and cleaves a different type of terminal amino acid, in accordance with some embodiments of the technology described herein;

FIG. 3 shows an example of polypeptide sequencing in real-time by evaluating binding interactions of terminal amino acids with labeled affinity reagents and a labeled non-specific exopeptidase, in accordance with some embodiments of the technology described herein;

FIG. 4 shows an example of polypeptide sequencing in real-time by evaluating binding interactions of terminal and internal amino acids with labeled affinity reagents and a labeled non-specific exopeptidase, in accordance with some embodiments of the technology described herein;

FIG. 5A shows an illustrative system in which aspects of the technology described herein may be implemented, in accordance with some embodiments of the technology described herein;

FIGS. 5B-C shows components of the protein sequencing device 502 shown in FIG. 5A, in accordance with some embodiments of the technology described herein;

FIG. 6A is an example process for training a machine learning model for identifying amino acids, in accordance with some embodiments of the technology described herein;

FIG. 6B is an example process for using the machine learning model obtained from the process of FIG. 6A for identifying a polypeptide, in accordance with some embodiments of the technology described herein;

FIG. 7 is an example process for providing input to a machine learning model, in accordance with some embodiments of the technology described herein;

FIG. 8 is an example of an output obtained from a machine learning model for use in identifying a polypeptide, in accordance with some embodiments of the technology described herein;

FIG. 9A shows exemplary data that may be obtained from binding interactions of reagents with amino acids, in accordance with some embodiments of the technology described herein;

FIG. 9B shows an example data structure for arranging the data of FIG. 9A, in accordance with some embodiments of the technology described herein;

FIG. 10A shows a plot of clustered data points for identification of clusters of a machine learning model, in accordance with some embodiments of the technology described herein;

FIG. 10B shows a plot of clusters identified from the clustered data points of FIG. 10A, in accordance with some embodiments of the technology described herein;

FIG. 10C shows a plot of example Gaussian mixture models (GMM) for each of the clusters of FIG. 10A, in accordance with some embodiments of the technology described herein;

FIG. 11 is a structure of an exemplary convolutional neural network (CNN) for identifying amino acids, in accordance with some embodiments of the technology described herein;

FIG. 12 is a block diagram of an exemplary connectionist temporal classification (CTC)-fitted model for identifying amino acids, in accordance with some embodiments of the technology described herein;

FIG. 13 is a block diagram of an illustrative computing device that may be used to implement some embodiments of the technology described herein;

FIGS. 14A-14C depict an illustrative approach for identifying regions of interest (ROIs) by calculating wavelet coefficients for a signal trace, in accordance with some embodiments of the technology described herein;

FIG. 15 is a flowchart of a method of identifying ROIs using the wavelet approach outlined above, in accordance with some embodiments of the technology described herein;

FIGS. 16A-16B depict illustrative approaches for fitting data produced from known affinity reagents to a parameterized distribution, in accordance with some embodiments of the technology described herein;

FIGS. 17A-17B depict an approach in which pulse duration values are fit to a sum of three exponential functions, wherein each fitted distribution includes a common exponential function, in accordance with some embodiments of the technology described herein;

FIG. 18 depicts a number of signal traces representing data obtained by measuring light emissions from a sample well, according to some embodiments, in accordance with some embodiments of the technology described herein;

FIGS. 19A-19E depict a process of training a GMM-based machine learning model based on signal traces for three amino acids, in accordance with some embodiments of the technology described herein; and

FIGS. 20A-20D depict a two-step approach to identifying amino acids, in accordance with some embodiments of the technology described herein.

### DETAILED DESCRIPTION

The inventors have developed a protein identification system that uses machine learning techniques to identify proteins. In some embodiments, the protein identification system operates by: (1) collecting data about a polypeptide of a protein using a real-time protein sequencing device; (2) using a machine learning model and the collected data to identify probabilities that certain amino acids are part of the polypeptide at respective locations; and (3) using the identified probabilities, as a “probabilistic fingerprint” to identify the protein. In some embodiments, data about the polypeptide of the protein may be obtained using reagents that selectively bind with amino acids. As an example, the reagents and/or amino acids may be labelled with luminescent labels (e.g., luminescent molecules) that emit light in response to application of excitation energy. In this example, a protein sequencing device may apply excitation energy to a sample of a protein (e.g., a polypeptide) during binding interactions of reagents with amino acids in the sample. In some embodiments, one or more sensors in the sequencing device (e.g., a photodetector, an electrical sensor, and/or any other suitable type of sensor) may detect binding interactions. In turn, the data collected and/or derived from the detected light emissions may be provided to the machine learning model.

The inventors have recognized that some conventional protein identification systems require identification of each amino acid in a polypeptide to identify the polypeptide. However, it is difficult to accurately identify each amino acid in a polypeptide. For example, data collected from an interaction in which a first labeled reagent selectively binds with a first amino acid may not be sufficiently different from data collected from an interaction in which a second labeled reagent selectively binds with a second amino acid to differentiate between the two amino acids. The inventors have solved this problem by developing a protein identification system that, unlike conventional protein identification systems, does not require (but does not preclude) identification of each amino acid in the protein.

As referred to herein, a polypeptide may include a polypeptide of a protein, a modified version of a protein, a mutated protein, a fusion protein, or a fragment thereof. Some embodiments are not limited to a particular type of protein. A polypeptide may comprise one or more peptides (also referred to as “peptide fragments”).

Some embodiments described herein address all of the above-described issues that the inventors have recognized with conventional protein identification systems. However, it should be appreciated that not every embodiment described herein addresses every one of these issues. It should also be appreciated that embodiments of the technology described herein may be used for purposes other than addressing the above-discussed issues of conventional protein identification systems.

In some embodiments, the protein identification system may access data (e.g., by a sensor part of a sequencing device) for binding interactions (e.g., detected light emissions, electrical signals, and/or any other type of signals) of one or more reagents with amino acids of a polypeptide. The protein identification system may provide the accessed data (with or without pre-processing) as input to a machine learning model to obtain respective output. The output may indicate, for each of multiple locations in the polypeptide, one or more likelihoods that one or more respective amino acids is present at the location. In some embodiments, the one or more likelihoods that the one or more respective amino acids is present at the location includes a first likelihood that a first amino acid is present at the location; and a second likelihood that the second amino acid is present at the location. The multiple locations may include relative locations within the polypeptide (e.g., locations relative to other outputs) and/or absolute locations within the polypeptide. The output may identify, for example, for each of the multiple locations, probabilities of different types of amino acids being present at the location. The protein identification system may use the output of the machine learning model to identify the polypeptide.

In some embodiments, the protein identification system may be configured to identify the polypeptide by identifying a protein to which the polypeptide corresponds. For example, the protein identification system may match the polypeptide to a protein from a predetermined set of proteins (e.g., stored a database of known proteins). In some embodiments, the protein identification system may be configured to identify a protein to which the polypeptide corresponds by matching the obtained output to one of multiple amino acid sequences associated with respective proteins. As an example, the protein identification system may match the output to an amino acid sequence stored in the UniProt database and/or the Human Proteome Project (HPP) database. In some embodiments, the protein identification system may be configured to match the output to an amino acid sequence by (1) generating a hidden Markov model (HMM) based on the output obtained from the machine learning model; and (2) matching the HMM to the amino acid sequence. As an example, the protein identification system may identify an amino acid sequence from the UniProt database that the HMM most closely aligns with as the matched amino acid sequence. The matched amino acid sequence may specify a protein of which the polypeptide forms a part. In some embodiments, the protein identification system may be configured to identify the polypeptide based on the output obtained from the machine learning model by matching the obtained output to multiple amino acid sequences in a database. For example, the protein identification system may determine that the output obtained from the machine learning model aligns with a first amino acid sequence and a second amino acid sequence in a database. In some embodiments, the protein identification system may be configured to identify the polypeptide based on the output obtained from the trained machine learning model by identifying likelihoods that the polypeptide aligns with respective one or more amino acid sequences in a database. For example, the protein identification system may determine that there is a 50% probability that the polypeptide aligns with a first amino acid sequence, and a 50% probability that the polypeptide aligns with a second amino acid sequence.

In some embodiments, the protein identification system may be configured to identify the polypeptide based on the output obtained from the trained machine learning model by eliminating one or more proteins that the polypeptide could be a part of. The protein identification system may be configured to determine, using the output obtained from the machine learning model, that it is not possible for the polypeptide to be part of one or more proteins, and thus eliminate the protein(s) from a set of candidate proteins. For example, the protein identification system may: (1) determine, using the output obtained from the machine learning model, that the polypeptide includes a set of one or more amino acids; and (2) eliminate amino acid sequences from a database (e.g., Uniprot and/or HPP) that do not include the set of amino acid(s).

In some embodiments, the protein identification system may be configured to identify the polypeptide by sequencing de novo to obtain a sequence of one or more portions (e.g., peptides) of the polypeptide. The protein identification system may be configured to use the output of the machine learning model to obtain a sequence of peptides of the polypeptide. In some embodiments, the protein identification system may be configured to identify the polypeptide based on the output obtained from the machine learning model by determining a portion or all of an amino acid sequence of the polypeptide. In some instances, the protein identification system may not identify an amino acid at one or more locations in the determined sequence. For example, the protein identification system may determine a portion or all of the amino acid sequence of the polypeptide where amino acids at one or more locations in the amino acid sequence are not identified. In some instances, the protein identification system may identify an amino acid at each location in the amino acid sequence or portion thereof. In some embodiments, the protein identification system may be configured to identify the polypeptide based on the output obtained from the machine learning model by determining multiple portions of an amino acid sequence of the polypeptide. In some instances, the protein identification system may determine non-contiguous portions of the amino acid sequence of the polypeptide. For example, the protein identification system may determine a first portion of the amino acid sequence, and a second portion of the amino acid sequence where the first portion is separated from the second portion by at least one amino acid in the amino acid sequence. In some instances, the protein identification system may determine contiguous portions of the amino acid sequence of the polypeptide. For example, the protein identification system may determine a first portion of the amino acid sequence and a second portion of the amino acid sequence where the first and second portions are contiguous. In some instances, the protein identification system may determine both contiguous and non-contiguous portions of an amino acid sequence of the polypeptide. For example, the protein identification system may determine three portion of the amino acid sequence where: (1) the first and second portions are contiguous portions; and (2) a third portion is separated from the first and second portions by a least one amino acid in the amino acid sequence.

In some embodiments, the protein identification system may be configured to obtain the sequence of peptides by identifying a natural pattern of amino acid sequences that occur in the polypeptide. For example, the protein identification system may be configured to determine that an identified amino acid sequence conforms to a natural patterns of amino acid sequences (e.g., in a database). In some embodiments, the protein identification system may be configured to obtain the sequence of peptides by identifying a learned pattern of amino acids. For example, the protein identification system may learn patterns of amino acids from one or more protein databases (e.g., Uniprot database and/or HPP database). The protein identification system may be configured to learn which peptides amino acid sequence patterns are likely to occur in, and use the information to obtain the sequence of peptides.

In some embodiments, the machine learning model may be configured to output, for each of multiple locations in a polypeptide, a probability distribution indicating, for each of multiple amino acids, a probability that the amino acid is present at the location. As an example, the machine learning model may output, for each of fifteen locations in the polypeptide, probabilities that each of twenty different amino acids is present at the location in the polypeptide. In some embodiments, the locations in the polypeptide for which the machine learning model is configured to generate an output may not necessarily correspond to actual locations in an amino acid sequence of the polypeptide. As an example, the first location for which the machine learning model generates an output may correspond to a second location in an amino acid sequence of the polypeptide, and a second location for which the machine learning model generates an output may correspond to a fifth amino acid location in the amino acid sequence of the polypeptide.

In some embodiments, data describing binding interactions of reagent(s) with amino acids of the polypeptide may include a plurality of light intensity values (e.g., values measured over time). Data indicating such measured light intensity values over time is referred to herein as a “signal trace,” and illustrative examples of signal traces are described further below. In some cases, the data describing binding interactions of reagent(s) with amino acids of the polypeptide may include values describing properties of a signal trace, such as one or more light pulse durations, pulse widths, pulse intensities, inter-pulse duration, or combinations thereof. For instance, a pulse duration value may indicate a duration of a signal pulse detected for a binding interaction of a reagent with an amino acid, whereas an inter-pulse duration value may indicate a duration of time between consecutive signal pulses detected for a binding interaction.

In some embodiments, the protein identification system may be configured to identify one or more proteins and/or polypeptides as follows. Initially, data describing binding interactions of reagent(s) with amino acids of the protein/polypeptide may be input to the trained machine learning model by: (1) identifying a plurality of portions of the data, each portion corresponding to a respective one of the binding interactions; and (2) providing each one of the plurality of portions as input to the trained machine learning model to obtain an output corresponding to the portion. Output produced by the machine learning model that corresponds to each portion of data may indicate one or more likelihoods that one or more respective amino acids is present at a respective location in a polypeptide. The output may in some cases indicate likelihoods for a single location within the polypeptide based on a single portion of the data. In other cases, the output may indicate that a single portion of the data is associated with more than one location within the polypeptide, either because there are consecutive identical amino acids represented by the portion (e.g., homopolymer), or because multiple indistinguishable amino acids may be represented by the portion. In the latter case, the output may comprise a probabilistic uncertainty in the specific number and/or identity of the amino acids in the polypeptide at the more than one location. With respect to the case of consecutive identical amino acids, it will be appreciated that sometimes the output may not explicitly indicate that a single portion of the data is associated with more than one location within the polypeptide, as in at least some cases it may not be possible to distinguish between a portion of the data that corresponds to two or more indistinguishable amino acids versus a portion of the data that corresponds to a single amino acid.

In some embodiments, the protein identification system may be configured to identify the plurality of portions of the data that each corresponds to one of the binding interactions, as follows: (1) identifying one or more points in the data corresponding to cleavage of one or more of the amino acids (e.g., from a polypeptide); and (2) identifying the plurality of portions of the data based on the identified one or more points corresponding to the cleavage of the one or more amino acids. In some embodiments, the protein identification system may be configured to identify the plurality of portions of the data by: (1) determining, from the data, a value of a summary statistic for one or more properties of the binding interactions (e.g., pulse duration, inter-pulse duration, luminescence intensity, and/or luminescence lifetime) by the luminescent labels; (2) identifying one or more points in the data at which a value of the at least one property deviates from the value of the summary statistic (e.g., mean) by a threshold amount; and identifying the plurality of portions of the data based on the identified one or more points.

In some embodiments, the data for the binding interactions of reagent(s) with amino acids of the polypeptide may include detected light emissions by one or more luminescent labels (e.g., that result from the binding interactions). In some embodiments, the luminescent label(s) may be associated with the reagent(s). As an example, the luminescent label(s) may be molecules that are linked to the reagent(s). In some embodiments, the luminescent label(s) may be associated with at least some amino acids of the polypeptide. As an example, the luminescent label(s) may be molecules that are linked to one or more classes of amino acids.

In some embodiments, the data for the binding interactions may be generated during the interactions. For example, a sequencing device sensor may detect the binding interactions as they occur, and generate the data from the detected interactions. In some embodiments, the data for the binding interactions may be generated before and/or after the interactions. For example, a sequencing device sensor may collect information before and/or after binding interactions occur, and generate the data using the collected information. In some embodiments, the data for the binding interactions may be generated before, during, and after the binding interactions.

In some embodiments, the data for the binding interactions may include luminescence intensity values and/or luminescence lifetime values of light emissions by the luminescent label(s). In some embodiments, the data may include wavelength values of light emissions by the luminescent label(s). In some embodiments, the data may include one or more light emission pulse duration values, one or more light emission inter-pulse duration values, one or more light emission luminescence lifetime values, one or more light emission luminescence intensity values, and/or one or more light emission wavelength values.

In some embodiments, luminescent labels may emit light in response to excitation light, which may for instance comprise a series of pulses of excitation light. As an example, a laser emitter may apply laser light that cause luminescent labels to emit light. Data collected from light emissions by the luminescent labels may include, for each of multiple pulses of excitation light, a respective number of photons detected in each of the plurality of time intervals, which are part of a time period after the pulse of excitation light. The data collected from light emissions may form a signal trace as discussed above.

In some embodiments, the protein identification system may be configured to arrange the data into a data structure to provide the data as input to a machine learning model. In some embodiments, the data structure may include: (1) a first column that holds a respective number of photons in each of a first and second time interval which are part of a first time period after a first light pulse in the series of light pulses; and (2) a second column that holds a respective number of photons in each of a first and second time interval which are part of a second time period after a second light pulse in the series of light pulses. In some embodiments, the data structure may include rows wherein each of the rows holds numbers of photons in a respective time interval corresponding to the light pulses. In some embodiments, the rows and columns may be interchanged. As an example, in some embodiments, the data structure may include: (1) a first column that holds a respective number of photons in each of a first and second time interval which are part of a first time period after a first light pulse in the series of light pulses; and (2) a second column that holds a respective number of photons in each of a first and second time interval which are part of a second time period after a second light pulse in the series of light pulses. In this example, the data structure may include columns where each of the columns holds numbers of photons in a respective time interval corresponding to the light pulses.

In some embodiments, the protein identification system may be configured to input data for binding interactions of reagent(s) with amino acids of the polypeptide into the trained machine learning model by arranging the data in an image, wherein each pixel of the image specifies a number of photons detected in a respective time interval of a time period after a light pulse of multiple light pulses. In some embodiments, the protein identification system may be configured to provide the data as input into the trained machine learning model by arranging the data in an image, wherein a first pixel of the image specifies a first number of photons detected in a first time interval of a first time period after a first pulse of multiple pulses. In some embodiments, a second pixel of the image specifies a second number of photons detected in a second time interval of the first time period after the first pulse of the multiple pulses. In some embodiments, a second pixel of the image specifies a second number of photons in a first time interval of a second time period after a second pulse of the multiple pulses.

In some embodiments, the data for binding interactions of reagent(s) with amino acids of the polypeptide may include electrical signals detected by an electrical sensor (e.g., an ammeter, a voltage sensor, etc.). As an example, a protein sequencing device may include one or more electrical sensors that detect electrical signals resulting from binding interactions of reagent(s) with amino acids of a polypeptide. The protein identification system may be configured to determine pulse duration values to be durations of electrical pulses detected for the binding interactions, and to determine inter-pulse durations values to be durations between consecutive electrical pulses detected for a binding interaction.

In some embodiments, the data for binding interactions of reagent(s) with amino acids of the polypeptide may be detected using a nanopore sensor. One or more probes (e.g., electrical probes) may be embedded in a nanopore. The probe(s) may detect signals (e.g., electrical signals) resulting from binding interactions of reagent(s) with amino acids of a polypeptide. As an example, the nanopore sensor may be a biological nanopore that measures voltage and/or electrical current changes resulting from binding interactions of reagent(s) with amino acids of the polypeptide. As another example, the nanopore sensor may be a solid state nanopore that measures voltage and/or electrical current changes resulting from binding interactions of reagent(s) with amino acids of the polypeptide. Examples of nanopore sensors are described in “Nano pore Sequencing Technology: A Review,” published in the International Journal of Advances in Scientific Research, Vol. 3, August 2017, and in “The Evolution of Nanopore Sequencing,” published in Frontiers in Genetics, Vol. 5, January 2015, both of which are incorporated herein by reference. In some embodiments, an affinity reagent may by a ClpS protein. For example, an affinity reagent may be a ClpS1 or ClpS2 protein from Agrobacterium tumefaciens or Synechococcus elongates. In another example, an affinity reagent may be a ClspS protein from Escherichia coli, Caulobacter crescentus, or Plasmodium falciparum. In some embodiments, an affinity reagent may be a nucleic acid aptamer.

It should be appreciated that aspects of the technology described herein are not limited to a particular technique of obtaining data for binding interactions of reagents with amino acids of a polypeptide, as the machine learning techniques described herein may be applied with data obtained through a variety of techniques.

In addition to the protein identification system described above, embodiments of a system for training a machine learning model for use in identifying a protein are also described herein. The training system may be configured to access training data obtained for binding interactions of one or more reagents with amino acids. The training system may train a machine learning model using the training data to obtain a trained machine learning model for identifying amino acids of polypeptides. Wherein the trained machine learning model is provided to a protein identification system as described above, the protein identification system and the training system may be the same system, or may be different systems.

In some embodiments, the training system may be configured to train the machine learning model by applying a supervised learning to the training data. As an example, training data may be input to the training system wherein each of multiple sets of data is labelled with an amino acid involved in a binding interaction corresponding to the set of data. In some embodiments, the training system may be configured to train the machine learning model by applying an unsupervised training algorithm to the training data. As an example, the training system may identify clusters for classification of data. Each of the clusters may be associated with one or more amino acids. In some embodiments, the training system may be configured to train the machine learning model by applying a semi-supervised learning algorithm to the training data. An unsupervised learning algorithm may be used to label unlabeled training data. The labelled training data may then be used to train the machine learning model by applying a supervised learning algorithm to the labelled training data.

In some embodiments, training data may include one or more pulse duration values, one or more inter-pulse duration values, and/or one or more luminescence lifetime values.

In some embodiments, the machine learning model may include multiple groups (e.g., clusters or classes), each associated with one or more amino acids. The training system may be configured to train a machine learning model for each class to distinguish between amino acid(s) of the class. As an example, the training system may train a mixture model (e.g., a Gaussian mixture model (GMM)) for each of the classes that represents multiple different amino acids associated with the class. The machine learning model may classify data into a class, and then output an indication of likelihoods that each of the amino acids associated with the class was involved in a binding interaction represented by the data. In some embodiments, the machine learning model may comprise a clustering model, wherein each class is defined by a cluster of the clustering model. Each of the clusters of the clustering model may be associated with one or more amino acids.

In some embodiments, the machine learning model may be, or may include, a deep learning model. In some embodiments, the deep learning model may be a convolution neural network (CNN). As an example, the convolution neural network may be trained to identify an amino acid based on a set of input data. In some embodiments, the deep learning model may be a connectionist temporal classification (CTC)-fitted neural network. The CTC-fitted neural network may be trained to output an amino acid sequence based on a set of input data. As an example, the CTC-fitted neural network may output a sequence of letters identifying the amino acid sequence.

In some embodiments, the training system may be configured to train the machine learning model based on data describing binding interactions of reagent(s) with amino acids of the polypeptide by: (1) identifying a plurality of portions of the data, each portion corresponding to a respective one of the binding interactions; (2) providing each one of the plurality of portions as input to the machine learning model to obtain an output corresponding to the each one portion of data; and (3) training the machine learning model using outputs corresponding to the plurality of portions. In some embodiments, the output corresponding to the portion of data indicates one or more likelihoods that one or more respective amino acids is present at a respective one of the plurality of locations.

In some embodiments, the training data obtained for binding interactions of reagent(s) with amino acids comprises data from detected light emissions by one or more luminescent labels. In some embodiments, the luminescent label(s) may be associated with the reagent(s). As an example, the luminescent label(s) may be molecules that are linked to the reagent(s). In some embodiments, the luminescent label(s) may be associated with at least some amino acids. As an example, the luminescent label(s) may be molecules that are linked to one or more classes of amino acids.

In some embodiments, the training data obtained from detected light emissions by luminescent labels may include luminescence lifetime values, luminescence intensity values, and/or wavelength values. A wavelength value may indicate a wavelength of light emitted by a luminescent label (e.g., during a binding interaction). In some embodiments, the light emissions are responsive to a series of light pulses, and the data includes, for each of at least some of the light pulses, a respective number of photons (also referred to as “counts”) detected in each of a plurality of time intervals which are part of a time period after the light pulse.

In some embodiments, the training system may be configured to train the machine learning model by providing the data as input to the machine learning model by arranging the data into a data structure having columns wherein: a first column holds a respective number of photons in each of a first and second time interval which are part of a first time period after a first light pulse in the series of light pulses; and a second column holds a respective number of photons in each of a first and second time interval which are part of a second time period after a second light pulse in the series of light pulses. In some embodiments, the training system may be configured to train the machine learning model by providing the data as input to the machine learning model by arranging the data into a data structure having rows wherein each of the rows holds numbers of photons in a respective time interval corresponding to the at least some light pulses. In some embodiments, the rows of the data structure may be interchanged with columns.

In some embodiments, the training system may be configured to provide the data as input into the machine learning model by arranging the data in an image, wherein each pixel of the image specifies a number of photons detected in a respective time interval of a time period after one of multiple light pulses. In some embodiments, the training system may be configured to provide the data as input to the machine learning model by arranging the data in an image, wherein a first pixel of the image specifies a first number of photons detected in a first time interval of a first time period after a first pulse of multiple light pulses. In some embodiments, a second pixel of the image specifies a second number of photons detected in a second time interval of the first time period after the first pulse of the multiple pulses. In some embodiments, a second pixel of the image specifies a second number of photons in a first time interval of a second time period after a second pulse of the multiple pulses.

In some embodiments, the training data for binding interactions of reagents with amino acids may include detected electrical signals detected by an electrical sensor (e.g., an ammeter, and/or a voltage sensor) for known proteins. As an example, a protein sequencing device may include one or more electrical sensors that detect electrical signals resulting from binding interactions of reagents with amino acids.

Some embodiments may not utilize machine learning techniques for identification of amino acids of a polypeptide. The protein identification system may be configured to access data for binding interactions of reagents with amino acids, and use the accessed data to identify a polypeptide. As an example, the protein identification system may use reagents that selectively bind to specific amino acids. The reagents may also be referred to as “tight-binding probes.” The protein identification system may use values of one or more properties (e.g., pulse duration, inter-pulse duration) of the binding interactions to identify an amino acid by determining which reagent was involved in a binding interaction. In some embodiments, the protein identification system may be configured to identify the amino acid by identifying a luminescent label associated with a reagent that selectively binds to the amino acid. As an example, the protein identification system may identify the amino acid using pulse duration values, and/or inter-pulse duration values. As another example, in embodiments in which the protein identification system detects light emissions of luminescent labels, the protein identification system may identify the amino acid using luminescent intensity values, and/or luminescent lifetime values of light emissions.

In some embodiments, the protein identification system may be configured to identify a first set of one or more amino acids using machine learning techniques and a second set of one or more amino acids without using machine learning techniques. In some embodiments, the protein identification system may be configured to use reagents that bind with multiple ones of the first set of amino acid(s). These reagents may be referred to herein as “weak-binding probes.” The protein identification system may be configured to use machine learning techniques described herein for identifying an amino acid from the first set. The protein identification system may be configured to use tight-binding probes for the second set of amino acid(s). The protein identification system may be configured to identify an amino acid from the second set without using machine learning techniques. As an example, the protein identification system may identify an amino acid from the second set based on pulse duration values, inter-pulse duration values, luminescent intensity values, luminescence lifetime values, wavelength values, and/or values derived therefrom.

Although the techniques are described herein primarily with reference to identification of proteins, in some embodiments, the techniques may be used for identification of nucleotides. As an example, the techniques described herein may be used to identify a DNA and/or RNA sample. The protein identification system may access data obtained from detected light emissions by luminescent labels during a degradation reaction in which affinity reagents are mixed with a nucleic acid sample that is to be identified. The protein identification system may provide the accessed data (with or without pre-processing) as input to a machine learning model to obtain a respective output. The output may indicate, for each of multiple locations in the nucleic acid, one or more likelihoods that one or more respective nucleotides was incorporated into the location of the nucleic acid. In some embodiments, the one or more likelihoods that the one or more respective nucleotides was incorporated at the location in the nucleic acid includes a first likelihood that a first nucleotide is present at the location; and a second likelihood that a second nucleotide is present at the location. As an example, the output may identify, for each of the multiple locations, probabilities of different nucleotides being present at the location. The protein identification system may use the output of the machine learning model to identify the nucleic acid.

In some embodiments, the protein identification system may be configured to match the obtained output to one of multiple nucleotide sequences associated with respective nucleic acids. As an example, the protein identification system may match the output to a nucleotide sequence stored in the GenBank database. In some embodiments, the protein identification system may be configured to match the output to match the output to a nucleotide sequence by (1) generating an HMM based on the output obtained from the machine learning model; and (2) matching the HMM to the nucleotide sequence. As an example, the protein identification system may identify a nucleotide sequence from the GenBank database that the HMM most closely aligns with as the matched nucleotide sequence. The matched nucleotide sequence may specify an identity of the nucleic acid to be identified.

Sequencing with Reagents

As discussed above, the protein identification system may be configured to identify one or more proteins and/or polypeptides based on data describing binding interactions of reagent(s) with amino acids of the proteins and/or polypeptides. In this section, an illustrative approach for producing such data is described.

In some embodiments, a polypeptide may be contacted with a labeled affinity reagent that selectively binds one or more types of amino acids. An affinity reagent may also be referred to herein as a “reagent.” In some embodiments, labeled affinity reagents may selectively bind with terminal amino acids. As used herein, in some embodiments, a terminal amino acid may refer to an amino-terminal amino acid of a polypeptide or a carboxy-terminal amino acid of a polypeptide. In some embodiments, a labeled affinity reagent selectively binds one type of terminal amino acid over other types of terminal amino acids. In some embodiments, a labeled affinity reagent selectively binds one type of terminal amino acid over an internal amino acid of the same type. In yet other embodiments, a labeled affinity reagent selectively binds one type of amino acid at any position of a polypeptide, e.g., the same type of amino acid as a terminal amino acid and an internal amino acid.

As used herein, a “type” of amino acid may refer to one of the twenty naturally occurring amino acids, a subset of types thereof, a modified variant of one of the twenty naturally occurring amino acids, or a subset of unmodified and/or modified variants thereof. Examples of modified amino acid variants include, without limitation, post-translationally-modified variants, chemically modified variants, unnatural amino acids, and proteinogenic amino acids such as selenocysteine and pyrrolysine. In some embodiments, a subset of types of amino acids may include more than one and fewer than twenty amino acids having one or more similar biochemical properties. As an example, in some embodiments, a type of amino acid refers to one type selected from amino acids with charged side chains (e.g., positive and/or negatively charged side chains), amino acids with polar side chains (e.g., polar uncharged side chains), amino acids with nonpolar side chains (e.g., nonpolar aliphatic and/or aromatic side chains), and amino acids with hydrophobic side chains.

In some embodiments, data is collected from detected light emissions (e.g., luminescence) of a luminescent label of an affinity reagent. In some embodiments, a labeled or tagged affinity reagent comprises (1) an affinity reagent that selectively binds with one or more types of amino acids; and (2) a luminescent label having a luminescence that is associated with the affinity reagent. In this way, the luminescence (e.g., luminescence lifetime, luminescence intensity, and other light emission properties described herein) may characteristic of the selective binding of the affinity reagent to identify an amino acid of a polypeptide. In some embodiments, a plurality of types of labeled affinity reagents may be used, wherein each type comprises a luminescent label having a luminescence that is uniquely identifiable from among the plurality. Suitable luminescent labels may include luminescent molecules, such as fluorophore dyes.

In some embodiments, data is collected from detected light emissions (e.g., luminescence) of a luminescent label of an amino acid. In some embodiments, a labeled amino acid comprises (1) an amino acid; and (2) a luminescent label having a luminescence that is associated with the amino acid. The luminescence may be used to identify an amino acid of a polypeptide. In some embodiments, a plurality of types of amino acids may be labeled, where each luminescent label has a luminescence that is uniquely identifiable from among the plurality of types.

As used herein, the terms “selective” and “specific” (and variations thereof, e.g., selectively, specifically, selectivity, specificity) may refer to a preferential binding interaction. As an example, in some embodiments, a labeled affinity reagent that selectively binds one type of amino acid preferentially binds the one type over another type of amino acid. A selective binding interaction will discriminate between one type of amino acid (e.g., one type of terminal amino acid) and other types of amino acids (e.g., other types of terminal amino acids), typically more than about 10- to 100-fold or more (e.g., more than about 1,000- or 10,000-fold). In some embodiments, a labeled affinity reagent selectively binds one type of amino acid with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M) without significantly binding to other types of amino acids. In some embodiments, a labeled affinity reagent selectively binds one type of amino acid (e.g., one type of terminal amino acid) with a K_{D} of less than about 100 nM, less than about 50 nM, less than about 25 nM, less than about 10 nM, or less than about 1 nM. In some embodiments, a labeled affinity reagent selectively binds one type of amino acid with a K_{D} of about 50 nM.

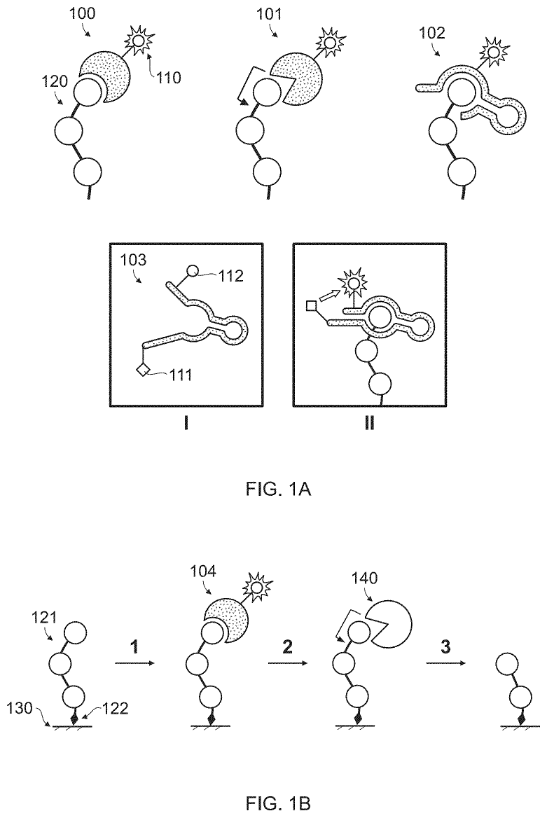

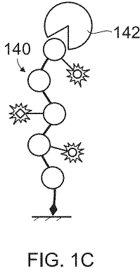

FIG. 1A shows various example configurations and uses of labeled affinity reagents, in accordance with some embodiments of the technology described herein. In some embodiments, a labeled affinity reagent 100 comprises a luminescent label 110 (e.g., a label) and an affinity reagent (shown as stippled shapes) that selectively binds one or more types of terminal amino acids of a polypeptide 120. In some embodiments, an affinity reagent may be selective for one type of amino acid or a subset (e.g., fewer than the twenty common types of amino acids) of types of amino acids at a terminal position or at both terminal and internal positions.

As described herein, an affinity reagent may be any biomolecule capable of selectively or specifically binding one molecule over another molecule (e.g., one type of amino acid over another type of amino acid). Affinity reagents include, as an example, proteins and nucleic acids. In some embodiments, an affinity reagent may be an antibody or an antigen-binding portion of an antibody, or an enzymatic biomolecule, such as a peptidase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.” A peptidase, also referred to as a protease or proteinase, may be an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. In some embodiments, an affinity reagent may be an N-recognin involved in an N-degron pathway in prokaryotes and eukaryotes as described in “The N-end rule pathway: From Recognition by N-recognins, to Destruction by AAA+Proteases,” published in Biochimica et Biophysica Acta (BBA)-Molecular Cell Research, Vol. 1823, Issue 1, January 2012.

In some embodiments, labeled affinity reagent 100 comprises a peptidase that has been modified to inactivate exopeptidase or endopeptidase activity. In this way, labeled affinity reagent 100 selectively binds without also cleaving the amino acid from a polypeptide. In some embodiments, a peptidase that has not been modified to inactivate exopeptidase or endopeptidase activity may be used. As an example, in some embodiments, a labeled affinity reagent comprises a labeled exopeptidase 101.

In some embodiments, protein sequencing methods may comprise iterative detection and cleavage at a terminal end of a polypeptide. In some embodiments, labeled exopeptidase 101 may be used as a single reagent that performs both steps of detection and cleavage of an amino acid. As generically depicted, in some embodiments, labeled exopeptidase 101 has aminopeptidase or carboxypeptidase activity such that it selectively binds and cleaves an N-terminal or C-terminal amino acid, respectively, from a polypeptide. It should be appreciated that, in certain embodiments, labeled exopeptidase 101 may be catalytically inactivated by one skilled in the art such that labeled exopeptidase 101 retains selective binding properties for use as a non-cleaving labeled affinity reagent 100, as described herein. In some embodiments, a labeled affinity reagent comprises a label having binding-induced luminescence. A binding interaction of the labeled affinity reagent with an amino acid may induce luminescence of a luminescent label that the reagent is labelled with.

In some embodiments, sequencing may involve subjecting a polypeptide terminus to repeated cycles of terminal amino acid detection and terminal amino acid cleavage. As an example, a protein sequencing device may collect data about an amino acid sequence of a polypeptide by contacting a polypeptide with one or more labeled affinity reagents.

FIG. 1B shows an example of sequencing using labeled affinity reagents, in accordance with some embodiments of the technology described herein. In some embodiments, sequencing comprises providing a polypeptide 121 that is immobilized to a surface 130 of a solid support (e.g., immobilized to a bottom or sidewall surface of a sample well) through a linker 122. In some embodiments, polypeptide 121 may be immobilized at one terminus (e.g., an amino-terminal amino acid) such that the other terminus is free for detecting and cleaving of a terminal amino acid. Accordingly, in some embodiments, the reagents interact with terminal amino acids at the non-immobilized (e.g., free) terminus of polypeptide 121. In this way, polypeptide 121 remains immobilized over repeated cycles of detecting and cleaving. To this end, in some embodiments, linker 122 may be designed according to a desired set of conditions used for detecting and cleaving, e.g., to limit detachment of polypeptide 121 from surface 130 under chemical cleavage conditions.

In some embodiments, sequencing comprises a step (1) of contacting polypeptide 121 with one or more labeled affinity reagents that selectively bind one or more types of terminal amino acids. As shown, in some embodiments, a labeled affinity reagent 104 interacts with polypeptide 121 by selectively binding the terminal amino acid. In some embodiments, step (1) further comprises removing any of the one or more labeled affinity reagents that do not selectively bind the terminal amino acid (e.g., the free terminal amino acid) of polypeptide 121. In some embodiments, sequencing comprises a step (2) of removing the terminal amino acid of polypeptide 121. In some embodiments, step (2) comprises removing labeled affinity reagent 104 (e.g., any of the one or more labeled affinity reagents that selectively bind the terminal amino acid) from polypeptide 121.

In some embodiments, sequencing comprises a step (3) of washing polypeptide 121 following terminal amino acid cleavage. In some embodiments, washing comprises removing protease 140. In some embodiments, washing comprises restoring polypeptide 121 to neutral pH conditions (e.g., following chemical cleavage by acidic or basic conditions). In some embodiments, sequencing comprises repeating steps (1) through (3) for a plurality of cycles.

FIG. 1C shows an example of sequencing using a labeled protein sample, in accordance with some embodiments of the technology described herein. As illustrated in the example embodiment of FIG. 1C, the labeled protein sample comprises a polypeptide 140 with labeled amino acids. In some embodiments, the labeled polypeptide 140 comprises a polypeptide with one or more amino acids that are labelled with a luminescent label. In some embodiments, one or more types of amino acids of the polypeptide 140 may be labeled, while one or more other types of amino acids of the polypeptide 140 may not be labeled. In some embodiments, all the amino acids of the polypeptide 140 may be labeled.

In some embodiments, sequencing comprises detecting a luminescence of a labeled polypeptide, which is subjected to repeated cycles of contact with one or more reagents. In the example embodiment of FIG. 1C, the sequencing comprises a step of contacting the polypeptide 140 with a reagent 142 that binds to one or more amino acids of the polypeptide 140. As an example, the reagent 142 may interact with a terminal amino acid of the labeled polypeptide. In some embodiments, the sequencing comprises a step of removing the terminal amino acid after contacting the polypeptide 140 with the reagent 142. In some embodiments, the reagent 142 may cleave the terminal amino acid after making contact with the polypeptide 140. The interaction of the reagent 142 with a labeled amino acid of the polypeptide 142 gives rise to one or more light emissions (e.g., pulses) which may be detected by a protein sequencing device.

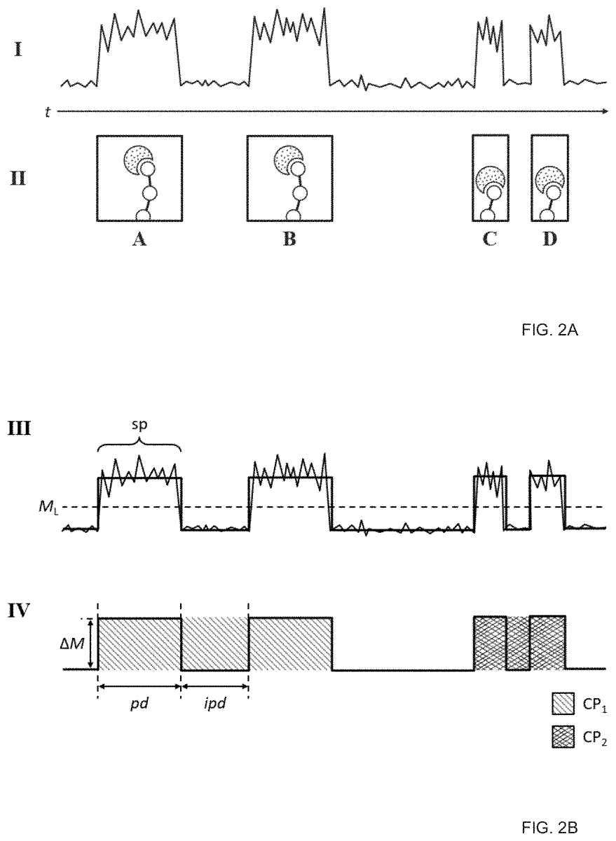

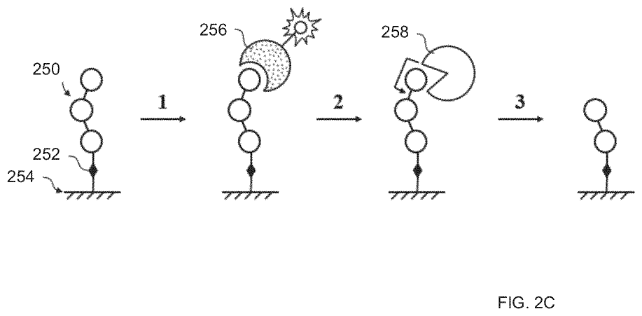

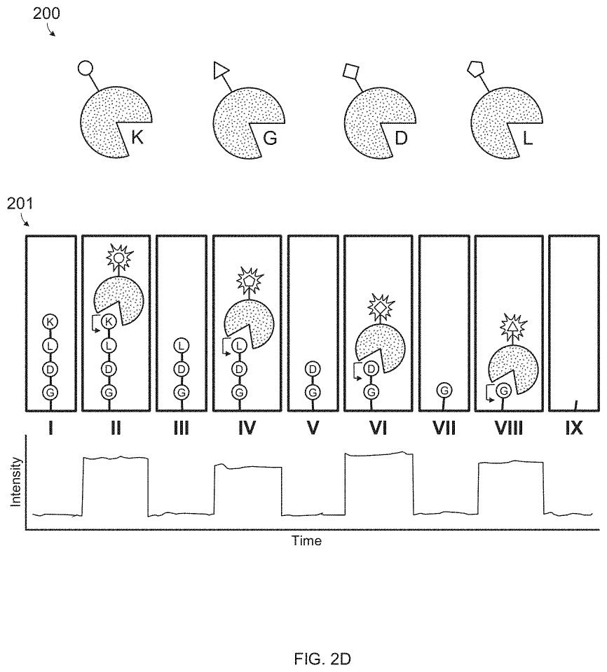

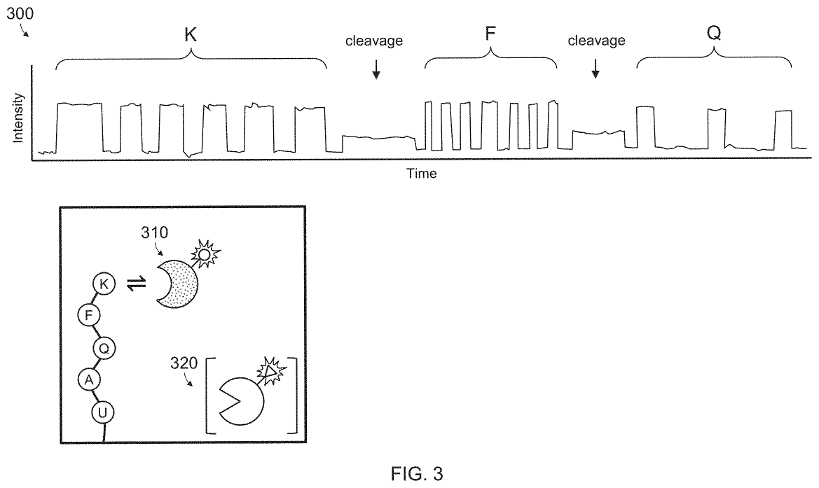

The above-described process of producing light emissions is further illustrated in FIG. 2A. An example signal trace (I) is shown with a series of panels (II) that depict different association events at times corresponding to changes in the signal. As shown, an association event between an affinity reagent (stippled shape) and an amino acid at the terminus of a polypeptide (shown as beads-on-a-string) produces a change in magnitude of the signal trace, being measurements of received excitation light, that persists for a duration of time.

As discussed above, an affinity reagent labeled with a luminescent label may emit light in response to excitation light being applied to the affinity reagent. When an affinity reagents associates with an amino acid, this light may be emitted proximate to the amino acid. If the affinity reagent subsequently is no longer associated with the amino acid, while its luminescent label may still emit light in response to excitation light, this light may be emitted from different spatial location and thereby may not be measured with the same intensity (or may not be measured at all) as the light emitted during association. As a result, by measuring light emitted from the amino acid, association events may be identified within the signal trace.

For instance, as shown in panels (A) and (B) of FIG. 2A, two different association events between an affinity reagent and a first amino acid exposed at the terminus of the polypeptide (e.g., a first terminal amino acid) each produce separate light emissions. Each association event produces a “pulse” of light, which is measured in the signal trace (I) and is characterized by a change in magnitude of the signal that persists for the duration of the association event. The time duration between the association events of panels (A) and (B) may correspond to a duration of time within which the polypeptide is not detectably associated with an affinity reagent.

Panels (C) and (D) depict different association events between an affinity reagent and a second amino acid exposed at the terminus of the polypeptide (e.g., a second terminal amino acid). As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids). Accordingly, the first and second amino acids of the series of panels (II) provide an illustrative example of successive amino acids exposed at the terminus of the polypeptide, where the second amino acid became the terminal amino acid upon removal of the first amino acid.

As generically depicted, the association events of panels (C) and (D) produce distinct light pulses, which are measured in the signal trace (I) and are characterized by changes in magnitude that persist for time durations that are relatively shorter than that of panels (A) and (B), and the time duration between the association events of panels (C) and (D) is relatively shorter than that of panels (A) and (B). As noted above, in some embodiments, such distinctive changes in signal may be used to determine characteristic patterns in the signal trace (I) which can discriminate between different types of amino acids.

In some embodiments, a transition from one characteristic pattern to another is indicative of amino acid cleavage. As used herein, in some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected. In some embodiments, signal pulse data can be analyzed as illustrated in FIG. 2B.

In some embodiments, a signal trace may be analyzed to extract signal pulse information by applying threshold levels to one or more parameters of the signal data. For example, panel (III) depicts a threshold magnitude level (“M_{L}”) applied to the signal data of the example signal trace (I). In some embodiments, M_{L} is a minimum difference between a signal detected at a point in time and a baseline determined for a given set of data. In some embodiments, a signal pulse (“sp”) is assigned to each portion of the data that is indicative of a change in magnitude exceeding M_{L} and persisting for a duration of time. In some embodiments, a threshold time duration may be applied to a portion of the data that satisfies M_{L} to determine whether a signal pulse is assigned to that portion. For example, experimental artifacts may give rise to a change in magnitude exceeding M_{L} that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a pulse may be identified from a signal trace based on a threshold magnitude level and a threshold time duration.

Extracted signal pulse information is shown in panel (III) with the example signal trace (I) superimposed for illustrative purposes. In some embodiments, a peak in magnitude of a signal pulse is determined by averaging the magnitude detected over a duration of time that persists above M_{L}. It should be appreciated that, in some embodiments, a “signal pulse,” or “pulse” as used herein can refer to a change in signal data that persists for a duration of time above a baseline (e.g., raw signal data, as illustrated by the example signal trace (I)), or to signal pulse information extracted therefrom (e.g., processed signal data, as illustrated in panel (IV)).

Panel (IV) shows the pulse information extracted from the example signal trace (I). In some embodiments, signal pulse information can be analyzed to identify different types of amino acids in a sequence based on different characteristic patterns in a series of signal pulses. For example, as shown in panel (IV), the signal pulse information is indicative of a first type of amino acid based on a first characteristic pattern (“CP₁”) and a second type of amino acid based on a second characteristic pattern (“CP₂”). By way of example, the two signal pulses detected at earlier time points provide information indicative of the first amino acid at the terminus of the polypeptide based on CP₁, and the two signal pulses detected at later time points provide information indicative of the second amino acid at the terminus of the polypeptide based on CP₂.

Also as shown in panel (IV), each signal pulse comprises a pulse duration (“pd”) corresponding to an association event between the affinity reagent and the amino acid of the characteristic pattern. In some embodiments, the pulse duration is characteristic of a dissociation rate of binding. Also as shown, each signal pulse of a characteristic pattern is separated from another signal pulse of the characteristic pattern by an interpulse duration (“ipd”). In some embodiments, the interpulse duration is characteristic of an association rate of binding. In some embodiments, a change in magnitude (“ΔM”) can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, as illustrated by FIGS. 2A-2B, in some embodiments, polypeptide sequencing may be performed by detecting a series of signal pulses produced by light emission from association events between affinity reagents labeled with luminescent labels. The series of signal pulses can be analyzed to determine characteristic patterns in the series of signal pulses, and the time course of characteristic patterns can be used to determine an amino acid sequence of the polypeptide.

In some embodiments, a protein or polypeptide can be digested into a plurality of smaller polypeptides and sequence information can be obtained from one or more of these smaller polypeptides (e.g., using a method that involves sequentially assessing a terminal amino acid of a polypeptide and removing that amino acid to expose the next amino acid at the terminus). In some embodiments, methods of peptide sequencing may involve subjecting a polypeptide terminus to repeated cycles of terminal amino acid detection and terminal amino acid cleavage.

A non-limiting example of polypeptide sequencing by iterative terminal amino acid detection and cleavage is depicted in FIG. 2C. In some embodiments, polypeptide sequencing comprises providing a polypeptide 250 that is immobilized to a surface 254 of a solid support (e.g., attached to a bottom or sidewall surface of a sample well) through a linkage group 252. In some embodiments, linkage group 252 is formed by a covalent or non-covalent linkage between a functionalized terminal end of polypeptide 250 and a complementary functional moiety of surface 254. For example, in some embodiments, linkage group 252 is formed by a non-covalent linkage between a biotin moiety of polypeptide 250 (e.g., functionalized in accordance with the disclosure) and an avidin protein of surface 254. In some embodiments, linkage group 252 comprises a nucleic acid.

In some embodiments, polypeptide 250 is immobilized to surface 254 through a functionalization moiety at one terminal end such that the other terminal end is free for detecting and cleaving of a terminal amino acid in a sequencing reaction. Accordingly, in some embodiments, the reagents used in certain polypeptide sequencing reactions preferentially interact with terminal amino acids at the non-immobilized (e.g., free) terminus of polypeptide 250. In this way, polypeptide 250 remains immobilized over repeated cycles of detecting and cleaving. To this end, in some embodiments, linkage group 252 may be designed according to a desired set of conditions used for detecting and cleaving, e.g., to limit detachment of polypeptide 250 from surface 254. Suitable linker compositions and techniques for functionalizing polypeptides (e.g., which may be used for immobilizing a polypeptide to a surface) are described in detail elsewhere herein.

In some embodiments, as shown in FIG. 2C, polypeptide sequencing can proceed by (1) contacting polypeptide 250 with one or more affinity reagents that associate with one or more types of terminal amino acids. As shown, in some embodiments, a labeled affinity reagent 256 interacts with polypeptide 250 by associating with the terminal amino acid.

In some embodiments, the method further comprises identifying the amino acid (terminal or internal amino acid) of polypeptide 250 by detecting labeled affinity reagent 256. In some embodiments, detecting comprises detecting a luminescence from labeled affinity reagent 256. In some embodiments, the luminescence is uniquely associated with labeled affinity reagent 256, and the luminescence is thereby associated with the type of amino acid to which labeled affinity reagent 256 selectively binds. As such, in some embodiments, the type of amino acid is identified by determining one or more luminescence properties of labeled affinity reagent 256.

In some embodiments, polypeptide sequencing proceeds by (2) removing the terminal amino acid by contacting polypeptide 250 with an exopeptidase 258 that binds and cleaves the terminal amino acid of polypeptide 250. Upon removal of the terminal amino acid by exopeptidase 258, polypeptide sequencing proceeds by (3) subjecting polypeptide 250 (having n−1 amino acids) to additional cycles of terminal amino acid recognition and cleavage. In some embodiments, steps (1) through (3) occur in the same reaction mixture, e.g., as in a dynamic peptide sequencing reaction. In some embodiments, steps (1) through (3) may be carried out using other methods known in the art, such as peptide sequencing by Edman degradation.

Edman degradation involves repeated cycles of modifying and cleaving the terminal amino acid of a polypeptide, wherein each successively cleaved amino acid is identified to determine an amino acid sequence of the polypeptide. Referring to FIG. 2C, peptide sequencing by conventional Edman degradation can be carried out by (1) contacting polypeptide 250 with one or more affinity reagents that selectively bind one or more types of terminal amino acids. In some embodiments, step (1) further comprises removing any of the one or more labeled affinity reagents that do not selectively bind polypeptide 250. In some embodiments, step (2) comprises modifying the terminal amino acid (e.g., the free terminal amino acid) of polypeptide 250 by contacting the terminal amino acid with an isothiocyanate (e.g., PITC) to form an isothiocyanate-modified terminal amino acid. In some embodiments, an isothiocyanate-modified terminal amino acid is more susceptible to removal by a cleaving reagent (e.g., a chemical or enzymatic cleaving reagent) than an unmodified terminal amino acid.

In some embodiments, Edman degradation proceeds by (2) removing the terminal amino acid by contacting polypeptide 250 with an exopeptidase 258 that specifically binds and cleaves the isothiocyanate-modified terminal amino acid. In some embodiments, exopeptidase 258 comprises a modified cysteine protease. In some embodiments, exopeptidase 258 comprises a modified cysteine protease, such as a cysteine protease from Trypanosoma cruzi (see, e.g., Borgo, et al. (2015) Protein Science 24:571-579). In yet other embodiments, step (2) comprises removing the terminal amino acid by subjecting polypeptide 250 to chemical (e.g., acidic, basic) conditions sufficient to cleave the isothiocyanate-modified terminal amino acid. In some embodiments, Edman degradation proceeds by (3) washing polypeptide 250 following terminal amino acid cleavage. In some embodiments, washing comprises removing exopeptidase 258. In some embodiments, washing comprises restoring polypeptide 250 to neutral pH conditions (e.g., following chemical cleavage by acidic or basic conditions). In some embodiments, sequencing by Edman degradation comprises repeating steps (1) through (3) for a plurality of cycles.

In some embodiments, peptide sequencing can be carried out in a dynamic peptide sequencing reaction. In some embodiments, referring again to FIG. 2C, the reagents required to perform step (1) and step (2) are combined within a single reaction mixture. For example, in some embodiments, steps (1) and (2) can occur without exchanging one reaction mixture for another and without a washing step as in conventional Edman degradation. Thus, in this embodiments, a single reaction mixture comprises labeled affinity reagent 256 and exopeptidase 258. In some embodiments, exopeptidase 258 is present in the mixture at a concentration that is less than that of labeled affinity reagent 256. In some embodiments, exopeptidase 258 binds polypeptide 250 with a binding affinity that is less than that of labeled affinity reagent 256.

FIG. 2D shows an example of polypeptide sequencing using a set of labeled exopeptidases 200, wherein each labeled exopeptidase selectively binds and cleaves a different type of terminal amino acid.

As illustrated in the example of FIG. 2D, labeled exopeptidases 200 include a lysine-specific exopeptidase comprising a first luminescent label, a glycine-specific exopeptidase comprising a second luminescent label, an aspartate-specific exopeptidase comprising a third luminescent label, and a leucine-specific exopeptidase comprising a fourth luminescent label. In some embodiments, each of labeled exopeptidases 200 selectively binds and cleaves its respective amino acid only when that amino acid is at an amino- or carboxy-terminus of a polypeptide. Accordingly, as sequencing by this approach proceeds from one terminus of a peptide toward the other, labeled exopeptidases 200 are engineered or selected such that all reagents of the set will possess either aminopeptidase or carboxypeptidase activity.

As further shown in FIG. 2D, process 201 schematically illustrates a real-time sequencing reaction using labeled exopeptidases 200. Panels (I) through (IX) illustrate a progression of events involving iterative detection and cleavage at a terminal end of a polypeptide in relation to a signal trace shown below, and corresponding to, the event depicted in each panel. For illustrative purposes, a polypeptide is shown having an arbitrarily selected amino acid sequence of “KLDG . . . ” (proceeding from one terminus toward the other).

Panel (I) depicts the start of a sequencing reaction, wherein a polypeptide is immobilized to a surface of a solid support, such as a bottom or sidewall surface of a sample well. In some embodiments, sequencing methods in accordance with the application comprise single molecule sequencing in real-time. In some embodiments, a plurality of single molecule sequencing reactions are performed simultaneously in an array of sample wells. In such embodiments, polypeptide immobilization prevents diffusion of a polypeptide out of a sample well by anchoring the polypeptide within the sample well for single molecule analysis.

Panel (II) depicts a detection event, wherein the lysine-specific exopeptidase from the set of labeled affinity reagents 200 selectively binds the terminal lysine residue of the polypeptide. As shown in the signal trace below panels (I) and (II), the signal indicates on this binding event by displaying an increase in signal intensity, which may be detected a sensor (e.g., a photodetector). Panel (III) illustrates that, after selectively binding a terminal amino acid, a labeled peptidase cleaves the terminal amino acid. As a result, these components are free to diffuse away from an observation region for luminescence detection, which is reported in the signal output by a drop in signal intensity, as shown in the trace below panel (III). Panels (IV) through (IX) proceed analogously to the process as described for panels (I) through (III). That is, a labeled exopeptidase binds and cleaves a corresponding terminal amino acid to produce a corresponding increase and decrease, respectively, in signal output.

The examples of FIGS. 2A-2D include recognition of terminal amino acids, internal amino acids and modified amino acids. It may be appreciated that a signal trace may allow for recognition of any combination these types of amino acids as well as each type individually. For instance, a terminal amino acid and the following internal amino acid may interact with one or more affinity reagents simultaneously and produce light indicative of the pair of amino acids.

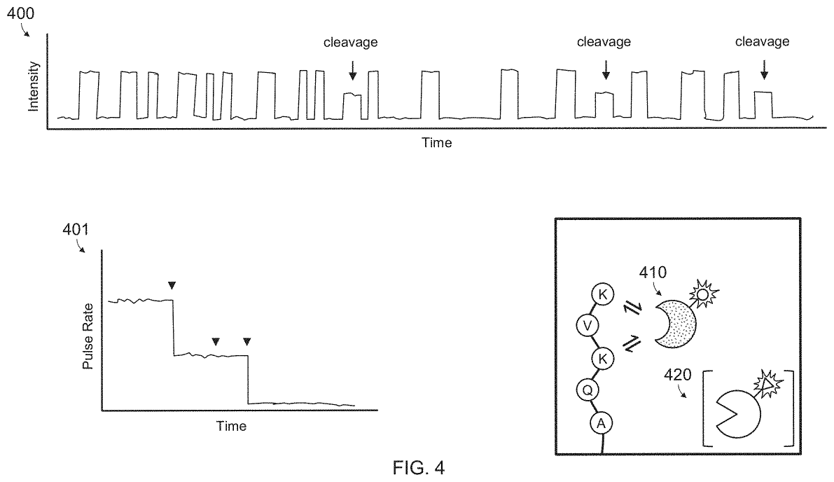

In some aspects, the application provides methods of polypeptide sequencing in real-time by evaluating binding interactions of terminal amino acids with affinity reagents and a labeled non-specific exopeptidase. In some embodiments, affinity reagents may be labeled (e.g., with a luminescent label). In some embodiments, affinity reagents may not be labeled. Example affinity reagents are described herein. FIG. 3 shows an example of a method of sequencing in which discrete binding events give rise to signal pulses of a signal trace 300. The inset panel of FIG. 3 illustrates a general scheme of real-time sequencing by this approach. As shown, a labeled affinity reagent 310 selectively binds to and dissociates from a terminal amino acid (shown here as lysine), which gives rise to a series of pulses in signal trace 300 which may be detected by a sensor. In some embodiments, the reagent(s) can be engineered to have target properties of binding. As an example, the reagents can engineered to achieve target values of pulse duration, inter-pulse duration, luminescence intensity, and/or luminescence lifetime.

Numbers of pulses, pulse duration values, and/or inter-pulse duration values described herein are for illustrative purposes. Some embodiments are not limited to particular numbers of pulses, pulse duration values, and/or inter-pulse duration values described herein. Further, amino acids described herein are for illustrative purposes. Some embodiments are not limited to any particular amino acid.

As shown in the inset panel, a sequencing reaction mixture further comprises a labeled non-specific exopeptidase 320 comprising a luminescent label that is different than that of labeled affinity reagent 310. In some embodiments, labeled non-specific exopeptidase 320 is present in the mixture at a concentration that is less than that of labeled affinity reagent 310. In some embodiments, labeled non-specific exopeptidase 320 displays broad specificity such that it cleaves most or all types of terminal amino acids.

As illustrated by the progress of signal trace 300, in some embodiments, terminal amino acid cleavage by labeled non-specific exopeptidase 320 gives rise to a signal pulse, and these events occur with lower frequency than the binding pulses of a labeled affinity reagent 310. As further illustrated in signal trace 300, in some embodiments, a plurality of labeled affinity reagents may be used, each with a diagnostic pulsing pattern, which may be used to identify a corresponding terminal amino acid.

FIG. 4 shows an example technique of sequencing in which the method described and illustrated for the approach in FIG. 3 is modified by using a labeled affinity reagent 410 that selectively binds to and dissociates from one type of amino acid (shown here as lysine) at both terminal and internal positions (FIG. 4, inset panel). As described in the previous approach, the selective binding gives rise to a series of pulses in signal trace 400. In this approach, however, the series of pulses occur at a rate that may be determined by the number of the type of amino acid throughout the polypeptide. Accordingly, in some embodiments, the rate of pulsing corresponding to binding events would be diagnostic of the number of cognate amino acids currently present in the polypeptide.

As in the previous approach, a labeled non-specific peptidase 420 would be present at a relatively lower concentration than labeled affinity reagent 410, e.g., to give optimal time windows in between cleavage events (FIG. 4, inset panel). In some embodiments, a uniquely identifiable luminescent label of labeled non-specific peptidase 420 may indicate when cleavage events have occurred. As the polypeptide undergoes iterative cleavage, the rate of pulsing corresponding to binding by labeled affinity reagent 410 would drop in a step-wise manner whenever a terminal amino acid is cleaved by labeled non-specific peptidase 420. This concept is illustrated by plot 401, which generally depicts pulse rate as a function of time, with cleavage events in time denoted by arrows. Thus, in some embodiments, amino acids may be identified—and polypeptides thereby sequenced—in this approach based on a pulsing pattern and/or on the rate of pulsing that occurs within a pattern detected between cleavage events.

### Machine Learning Techniques for Protein Identification

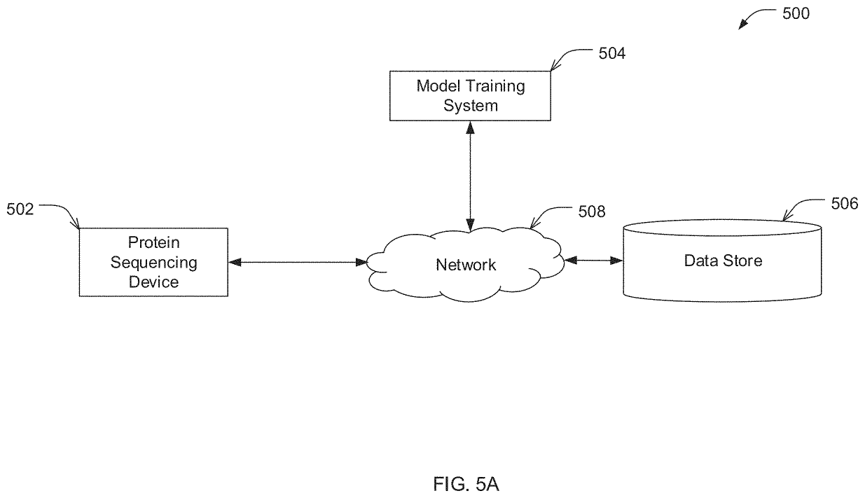

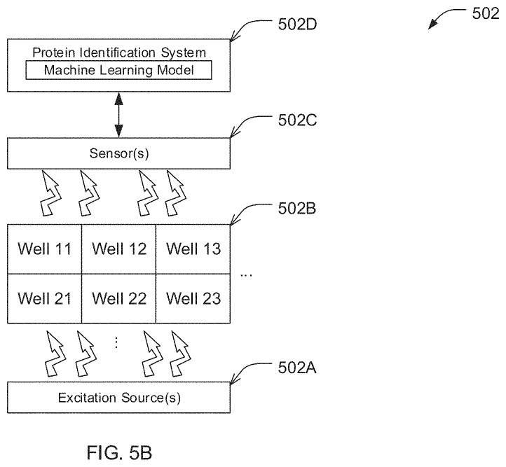

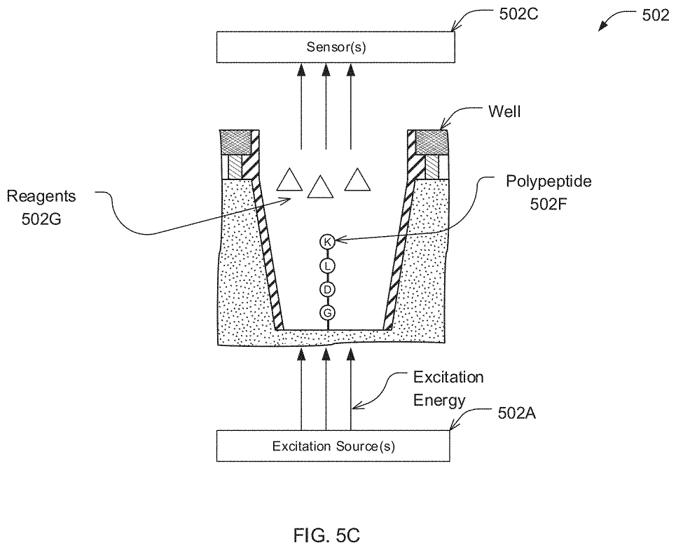

FIG. 5A shows a system 500 in which aspects of the technology described may be implemented. The system 500 includes a protein sequencing device 502, a model training system 504, and a data store 506, each of which is connected to a network 508.

In some embodiments, the protein sequencing device 502 may be configured to transmit data obtained from sequencing of polypeptides of proteins (e.g., as described above with reference to FIGS. 1-4) to the data store 506 for storage. Examples of data that may be collected by the protein sequencing device 502 are described herein. The protein sequencing device 502 may be configured to obtain a machine learning model from the model training system 504 via the network 508. In some embodiments, the protein sequencing device 502 may be configured to identify a polypeptide using the trained machine learning model. The protein sequencing device 502 may be configured to identify an unknown polypeptide by: (1) accessing data collected from amino acid sequencing of the polypeptide; (2) providing the data as input to the trained machine learning model to obtain an output; and (3) using the corresponding output to identify the polypeptide. Components of the protein sequencing device 502 are described herein with reference to FIGS. 5B-C.

Although the exemplary system 500 illustrated in FIG. 5A shows a single protein sequencing device, in some embodiments, the system 500 may include multiple protein sequencing devices.

In some embodiments, the model training system 504 may be a computing device configured to access the data stored in the data store 506, and use the accessed data to train a machine learning model for use in identifying polypeptides. In some embodiments, the model training system 504 may be configured to train a separate machine learning model for each of multiple protein sequencing devices. As an example, the model training system 504 may: (1) train a first machine learning model for a first protein sequencing device using data collected by the first protein sequencing device from amino acid sequencing; and (2) train a second machine learning model for a second protein sequencing device using data collected by the second protein sequencing device from amino acid sequencing. A separate machine learning model for each of the devices may be tailored to unique characteristics of the respective protein sequencing devices. In some embodiments, the model training system 504 may be configured to provide a single trained machine learning model to multiple protein sequencing devices. As an example, the model training system 504 may aggregate data collected from amino acid sequencing performed by multiple protein sequencing devices, and train a single machine learning model. The single machine learning model may be normalized for multiple protein sequencing devices to mitigate model parameters resulting from device variation.

In some embodiments, the model training system 504 may be configured to periodically update a previously trained machine learning model. In some embodiments, the model training system 504 may be configured to update a previously trained model by updating values of one or more parameters of the machine learning model using new training data. In some embodiments, the model training system 504 may be configured update the machine learning model by training a new machine learning model using a combination of previously-obtained training data and new training data.

The model training system 504 may be configured to update a machine learning model in response to any one of different types of events. For example, in some embodiments, the model training system 504 may be configured to update the machine learning model in response to a user command. As an example, the model training system 504 may provide a user interface via which the user may command performance of a training process. In some embodiments, the model training system 504 may be configured to update the machine learning model automatically (i.e., not in response to a user command), for example, in response to a software command. As another example, in some embodiments, the model training system 504 may be configured to update the machine learning model in response to detecting one or more conditions. For example, the model training system 504 may update the machine learning model in response to detecting expiration of a period of time. As another example, the model training system 504 may update the machine learning model in response to receiving a threshold amount of new training data.

In some embodiments, the model training system 504 may be configured to train the machine learning model by applying a supervised learning training algorithm to labelled training data. As an example, the model training system 504 may be configured to train a deep learning model (e.g., a neural network) by using stochastic gradient descent. As another example, the model training system 504 may train a support vector machine (SVM) to identify decision boundaries of the SVM by optimizing a cost function. In some embodiments, the model training system 504 may be configured to train the machine learning model by applying an unsupervised learning algorithm to training data. As an example, the model training system 504 may identify clusters of a clustering model by performing k-means clustering. In some embodiments, the model training system 504 may be configured to train the machine learning model by applying a semi-supervised learning algorithm to training data. As an example, the model training system 504 may (1) label a set of unlabeled training data by applying an unsupervised learning algorithm (e.g., clustering) to training data; and (2) applying a supervised learning algorithm to the labelled training data.

In some embodiments, the machine learning model may include a deep learning model (e.g., a neural network). As an example, the deep learning model may include a convolutional neural network (CNN), a recurrent neural network (RNN), a multi-layer perceptron, an autoencoder and/or a CTC-fitted neural network model. In some embodiments, the machine learning model may include a clustering model. As an example, the clustering model may include multiple clusters, each of the clusters being associated with one or more amino acids.

In some embodiments, the machine learning model may include one or more mixture models. The model training system 504 may be configured to train a mixture model for each of the groups (e.g., classes or groups) of the machine learning model. As an example, the machine learning model may include six different groups. The model training system 504 may train a Gaussian mixture model (GMM) for each of the groups. The model training system 504 may train a GMM for a respective group using training data for binding interactions involving amino acid(s) associated with the respective group. It should be appreciated that the foregoing examples of machine learning models are non-limiting examples and that any other suitable type of machine learning model may be used in other embodiments, as aspects of the technology described herein are not limited in this respect.

In some embodiments, the data store 506 may be a system for storing data. In some embodiments, the data store 506 may include one or more databases hosted by one or more computers (e.g., servers). In some embodiments, the data store 508 may include one or more physical storage devices. As an example, the physical storage device(s) may include one or more solid state drives, hard disk drives, flash drives, and/or optical drives. In some embodiments, the data store 506 may include one or more files storing data. As an example, the data store 506 may include one or more text files storing data. As another example, the data store 506 may include one or more XML files. In some embodiments, the data store 506 may be storage (e.g., a hard drive) of a computing device. In some embodiments, the data store 506 may be a cloud storage system.

In some embodiments, the network 508 may be a wireless network, a wired network, or any suitable combination thereof. As one example, the network 508 may be a Wide Area Network (WAN), such as the Internet. In some embodiments, the network 508 may be a local area network (LAN). The local area network may be formed by wired and/or wireless connections between the protein sequencing device 502, model training system 504, and the data store 506. Some embodiments are not limited to any particular type of network described herein.

FIG. 5B shows components of the protein sequencing device 502 shown in FIG. 5A, in accordance with some embodiments of the technology described herein. The protein sequencing device 502 includes one or more excitation sources 502A, one or more wells 502B, one or more sensors 502C, and a protein identification system 502D.

In some embodiments, the excitation source(s) 502A are configured to apply excitation energy (e.g., pulses of light) to multiple different wells 502B. In some embodiments, the excitation source(s) 502A may be one or more light emitters. As an example, the excitation source(s) 502A may include one or more laser light emitters that emit pulses of laser light. As another example, the excitation source(s) 502A may include one or more light emitting diode (LED) light sources that emit pulses of light. In some embodiments, the excitation source(s) 502A may be one or more devices that generate radiation. As an example, the excitation source(s) 502A may emit ultra violet (UV) rays.

In some embodiments, the excitation source(s) 502A may be configured to generate excitation pulses that are applied to the wells 502B. In some embodiments, the excitation pulses may be pulses of light (e.g., laser light). The excitation source(s) 502A may be configured to direct the excitation pulses the wells 502B. In some embodiments, the excitation source(s) 502A may be configured to repeatedly apply excitation pulses to a respective well. As an example, the excitation source(s) 502A may emit laser pulses at a frequency of 100 MHz. Application of a light pulse to a luminescent label may cause the luminescent label to emit light. As an example, the luminescent label may absorb one or more photons of applied light pulses and, in response, emit one or more photons. Different types of luminescent labels (e.g., luminescent molecules) may respond differently to application of excitation energy. As an example, different types of luminescent labels may release different numbers of photons in response to a pulse of light and/or release photons at different frequencies in response to a pulse of light.

In some embodiments, each of the well(s) 502B may include a container configured to hold one or more samples of a specimen (e.g., samples of protein polypeptides). In some embodiments, binding interactions of one or more reagents with amino acids of a polypeptide may take place in the well(s) 502B (e.g., as described above with reference to FIGS. 1-4). The reagent(s) may be labeled with luminescent labels. In response to the excitation energy applied by the excitation source(s) 502A, the luminescent labels may emit light.

As shown in the example embodiment of FIG. 5B, in some embodiments, the well(s) 502B may be arranged into a matrix of wells. Each well in the matrix may include a container configured to hold one or more samples of a specimen. In some embodiments, the well(s) 502B may be placed in an arrangement different from one illustrated in FIG. 5B. As an example, the well(s) 502B may be arranged radially around a central axis. Some embodiments are not limited to a particular arrangement of the well(s) 502B.

In some embodiments, the sensor(s) 502C may be configured to detect light emissions (e.g., by luminescent labels) from the well(s) 502B. In some embodiments, the sensor(s) 502C may be one or more photodetectors configured to convert the detected light emissions in to electrical signals. As an example, the sensor(s) 502C may convert the light emissions into an electrical voltage or current. The electrical voltage or current may further may converted into a digital signal. The generated signal may be used (e.g., by the protein identification system 502C) for identification of a polypeptide. In some embodiments, the signals generated by the sensor(s) 502C may be processed to obtain values of various properties of the light emissions. As an example, the signals may be processed to obtain values of intensities of light emission, duration of light emission, durations between light emissions, and lifetime of light emissions.

In some embodiments, the sensor(s) 502C may be configured to measure light emissions by luminescent labels over a measurement period. As an example, the sensor(s) 502C may measure a number of photons over a 10 ms measurement period. In some embodiments, a luminescent label may emit photons in response to excitation with a respective probability. As an example, a luminescent label may emit 1 photon in every 10,000 excitations. If the luminescent label is excited 1 million times within a 10 ms measurement period, approximately 100 photons may be detected by the sensor(s) 502C in this example. Different luminescent labels may emit photons with different probabilities. Some embodiments are not limited to any particular probability of photon emission described herein, as values described herein are for illustrative purposes.

In some embodiments, the sensor(s) 502C may be configured to determine the number of photons (a “photon count”) detected in each of multiple time intervals of a time period following application of an excitation pulse (e.g., a laser pulse). A time interval may also be referred to herein as an “interval”, a “bin” or a “time bin.” As an example, the sensor(s) 502C may determine the number of photons detected in a first time interval of approximately 3 ns after application of an excitation pulse, and the number of photons detected in a second interval of approximately 3 ns after application of the laser pulse. In some embodiments, the time intervals may have substantially the same duration. In some embodiments, the time intervals may have different durations. In some embodiments, the sensor(s) 502C may be configured to determine the number of detected photons in 2, 3, 4, 5, 6, or 7 time intervals of a time period following application of an excitation pulse. Some embodiments are not limited to any number of time intervals for which the sensor(s) 502C are configured to determine the number of detected photons.

In some embodiments, the protein identification system 502D may be a computing device configured to identify a polypeptide based on data collected by the sensor(s) 502C. The protein identification system 502D includes a machine learning model that is used by the protein identification system 502D for identifying a polypeptide. In some embodiments, the trained machine learning model may be obtained from the model training system 504 described above with reference to FIG. 5A. Examples of machine learning models that may be used by the protein identification system 502D are described herein. In some embodiments, the protein identification system 502D may be configured to generate an input to the machine learning model using data collected by the sensor(s) 502C to obtain an output for use in identifying a polypeptide.

In some embodiments, the protein identification system 502D may be configured to process data collected by the sensor(s) 502C to generate data to provide as input (with or without additional pre-processing) to the machine learning model. As an example, the protein identification system 502D may generate data to provide as input to the machine learning model by determining values of one or more properties of binding interactions detected by the sensor(s) 502C. Example properties of binding interactions are described herein. In some embodiments, the protein identification system 502D may be configured to generate data to provide as input to the machine learning model by arranging the data into a data structure (e.g., a matrix or image). As an example, the protein identification system 502D may identify photon counts detected in time intervals of time periods following application of one or more excitation pulses (e.g., laser pulses). The protein identification system 502D may be configured to arrange the photon counts into a data structure for inputting into the machine learning model. As an example, the protein identification system 502D may arrange the photon counts following excitation pulses into columns or rows of a matrix. As another example, the protein identification system 502D may generate an image for input into the machine learning model, wherein the pixels of the image specify respective photon counts.

In some embodiments, the protein identification system 502D may be configured to determine an indication of intensity of light emissions by a luminescent label, which may be referred to herein as “luminescence intensity.” The luminescence intensity may be the number of photons emitted per unit of time by a luminescent label in response to application of excitation energy (e.g., laser pulses). As an example, if the protein identification system 502D determines that 5 total photons were detected in a 10 ns measurement time period after application of an excitation pulse, the protein identification system 502D may determine the luminescence intensity value to be 0.5 photons/ns. In some embodiments, protein identification system 502D may be configured to determine an indication of luminescence intensity based on a total number of photons detected after application of each of multiple excitation pulses. In some embodiments, the protein identification system 502D may determine a mean number of photons detected after application of multiple excitation pulses to be the indication of luminescence intensity.

In some embodiments, the protein identification system 502D may be configured to determine an indication of a lifetime of light emissions by a luminescent label, which may be referred to herein as “luminescence lifetime.” The luminescence lifetime may be a rate at which probability of photon emission decays over time. As an example, if the protein identification system 502D determines a number of photons detected in two intervals of a time period after application of an excitation pulse, then the protein identification system 502D may determine a ratio of the number of photons in the second interval to the number of photons in the first interval to be an indication of decay of photon emissions over time.

In some embodiments, the protein identification system 502D may be configured to determine an indication of a duration of each of one or more signal pulses detected for a binding interaction of a reagent with an amino acid. A duration of a signal pulse may also be referred to herein as “pulse duration.” For example, during a binding interaction of a reagent with an amino acid, a luminescent label that the reagent and/or amino acid is labeled with may emit one or more pulses of light. In some embodiments, the protein identification system 502D may be configured to determine the duration of a light pulse to be a pulse duration value. As an example, FIG. 3 discussed above illustrates a series of pulses of light emitted during a binding interaction of a labeled reagent 310 with an amino acid (K). The protein identification system 502D may be configured to determine pulse duration values to be the durations of the pulses of light for the binding interaction involving the amino acid (K) shown in FIG. 3. In some embodiments, the protein identification system 502D may be configured to determine a pulse duration value to be a duration of an electrical pulse detected by an electrical sensor (e.g., a voltage sensor). Some embodiments are not limited to a particular technique of detecting pulse duration.

In some embodiments, the protein identification system 502D may be configured to determine an indication of a duration of time between consecutive signal pulses detected for a binding interaction of a reagent with an amino acid. A duration of time between consecutive signal pulses may also be referred to herein as “inter-pulse duration.” During each of the binding interactions, a luminescent label may emit multiple pulses of light. In some embodiments, the protein identification system 502D may be configured to determine an inter-pulse duration value to be a duration of time between two consecutive pulses of light. As an example, the protein identification system 502D may determine the inter-pulse duration values to be durations of time between the light pulses for the binding interaction of a reagent with amino acid (K) shown in FIG. 3. In some embodiments, the protein identification system 502D may be configured to determine an inter-pulse duration value to be a duration between electrical pulses detected by an electrical sensor (e.g., a voltage sensor). Some embodiments are not limited to a particular technique of detecting pulse duration.

In some embodiments, the protein identification system 502D may be configured to determine values of one or more parameters determined from one or more properties of binding interactions described herein. In some embodiments, the protein identification system 502D may be configured to determine a summary statistic across a set of values of a property. As an example, the system may determine a mean, median, standard deviation, and/or range of a set of pulse duration values, inter-pulse duration values, luminescence intensity values, luminescence lifetime values, and/or wavelength values. In some embodiments, the protein identification system 502D may be configured to determine a mean pulse duration value for a binding reaction. As an example, the protein identification system 502D may determine the mean pulse duration value of the binding interaction of amino acid (K) shown in FIG. 3 to be a mean duration of alight pulse emitted during the binding interaction. In some embodiments, the protein identification system 502D may be configured to determine a mean inter-pulse duration value for a binding reaction. As an example, the protein identification system 502D may determine the mean inter-pulse duration value for the binding interaction of amino acid (K) shown in FIG. 3 to be a mean of duration between consecutive light pulses emitted during the binding interaction. In some embodiments, the parameters may include properties of reagents and/or luminescent labels. In some embodiments, the properties may include kinetic constants of reagents and/or luminescent labels using values of the properties. As an example, the system may determine a binding affinity (K_{D}), an on rate of binding (kₒₙ), and/or an off rate of binding (k_{off}) using pulse duration and/or interpulse duration values.

In some embodiments, the protein identification system 502D may be configured to determine values indicating a ratio of pulse duration to inter-pulse duration, a ratio of luminescence lifetime to luminescence intensity, and/or any other value that can be determined from the values of the properties.

In some embodiments, the protein identification system 502D may be configured to obtain output from the trained machine learning model in response to a provided input. The protein identification system 502D may be configured to use the output to identify a polypeptide. In some embodiments, the output may indicate, for each of multiple locations in the polypeptide, one or more likelihoods that one or more amino acids are at the location in the polypeptide. As an example, the output may indicate, for each of the locations, a likelihood that each of twenty naturally occurring amino acids is present at the location. In some embodiments, the protein identification system 502D may be configured to normalize likelihoods may be normalized or un-normalized. In some embodiments, a normalized likelihood may be referred to as a “probability” or a “normalized likelihood.” In some embodiments the probabilities may sum to 1. For example, the likelihoods of four amino acids being present at a location may be 5, 5, 5 and 5. The probabilities (or normalized likelihoods) of this example may be 0.25, 0.25, 0.25, and 0.25.

In some embodiments, for each of the multiple locations in the polypeptide, the output may be a probability distribution indicating, for each of the amino acid(s), a probability that the amino acid is present at the location. The output may indicate a probability for each amino acid as a location relative to the other amino acids, or may indicate a probability for an absolute location of the amino acid within the polypeptide. For each location, for example, the output specifies a value for each of twenty amino acids indicating a probability that the amino acid is present at the location. In some embodiments, the protein identification system 502D may be configured to obtain an output that identifies an amino acid sequence of the polypeptide. As an example, the output of the machine learning model may be a sequence of letters identifying a chain of amino acids that form a portion of the polypeptide.

In some embodiments, the protein identification system 502D may be configured to use the output obtained from the machine learning model to identify the polypeptide. In some embodiments, the protein identification system 502D may be configured to match an output obtained from the machine learning model to a protein in a database of proteins. In some embodiments, the protein identification system 502D may access a data store of known amino acid sequences specifying respective proteins. The protein identification system 502D may be configured to match the output of the machine learning model to a protein by identifying an amino acid sequence from the data store that the output from the machine learning model best aligns with. As an example, when the output indicates likelihoods that various amino acids are present at locations in the polypeptide, the system may identify an amino acid sequence with which the output aligns with most closely from the sequences in the data store. The protein identification system 502D may identify the respective protein specified by the identified amino acid sequence to be the protein.

In some embodiments, the protein identification system 502D may be configured to generate a hidden Markov model (HMM) based on the obtained output from the machine learning system, and match the HMM against known amino acid sequences. The protein identification system 502D may identify the protein as the one associated with the amino acid sequence with which the HMM is matched. As another example, the output of the machine learning system may identify an amino acid sequence. The protein identification system 502D may select an amino acid sequence from the data store that most closely matches the amino acid sequence identified by the output of the machine learning system. The protein identification system 502D may determine the closet match by determining which known amino acid sequence has the fewest discrepancies from the amino acid sequence identified by the output of the machine learning system. The protein identification system 502D may identify the protein as one associated with the amino acid sequence selected from the data store.

In some embodiments, the protein identification system 502D may be configured to calibrate the protein sequencing device 502. In some embodiments, the protein identification system 502D may be configured to calibrate the protein sequencing device 502 by training the machine learning model. The protein identification system 502D may be configured to train the machine learning model using one or more of the approaches described with reference to the model training system 504.

In some embodiments, the protein identification system 502D may be configured to calibrate the protein sequencing device 502 by training the machine learning model using data associated with one or more known polypeptides (e.g., for which the amino acid sequence(s) are known either in part or in whole). By performing training with data associated with known polypeptide sequences, the protein identification system 502D may obtain a machine learning model that provides output that more accurately distinguishes between different amino acids and/or proteins. In some embodiments, the protein identification system 502D may be configured to use data obtained from detected light emissions by luminescent labels during binding interactions of reagents with amino acids of polypeptides for which the amino acid sequences are known either in part or in whole. In some embodiments, the protein identification system 502D may be configured to apply a training algorithm to the data to identify one or more groups (e.g., classes and/or clusters) that can be used by the machine learning model to generate an output.

In some embodiments, the machine learning model may include a clustering model, and the protein identification system 502D may be configured to calibrate the protein sequencing device 502 by applying an unsupervised learning algorithm (e.g., k-means) to identify clusters of the clustering model. The identified clusters may then be used by the machine learning model to generate outputs for use in identifying unknown polypeptides. As an example, the protein identification system 502D may identify centroids of the clusters, which may be used by the machine learning model to generate an output for data input to the machine learning model. As another example, the protein identification system 502D may identify boundaries between different groups of amino acids (e.g., based on pulse duration, inter-pulse duration, wavelength, luminescence intensity, luminescence lifetime, and/or any other value derived from these and/or other properties). A position of a data point relative to the boundaries may then be used by the machine learning model to generate an output for a respective input to the machine learning model.

In some embodiments, the protein identification system 502D may be configured to calibrate the protein sequencing device 502 for each of the wells 502B. The protein identification system 502D may be configured to train, for each individual well, a respective machine learning model using data obtained for binding interactions that have taken place in the individual well. This would provide a protein sequencing device 502 that is fine-tuned to individual wells 502B. In some embodiments, the protein identification system 502D may be configured to calibrate the protein sequencing device 502 for multiple wells. The protein identification system 502D may be configured to train a machine learning model using data obtained for binding interactions that have taken place across multiple wells of the sequencer. In some embodiments, the protein identification system 502D may be configured to obtain a generalized model that may be used for multiple wells. The generalized model may average or otherwise smooth out idiosyncrasies in the data obtained from an individual well and may have good performance across multiple wells, whereas a model tailored to a particular well may perform better on future data obtained from the particular well, but may not perform better on future data from multiple different wells.

In some embodiments, the protein identification system 502D may be configured to adapt, to a particular individual well, a generalized model created for multiple wells, by using data obtained from the individual well. As an example, the protein identification system 502D may modify cluster centroids of the generalized model for a respective well based on data obtained for binding interactions in the well.

Calibrating a single model for multiple wells may have the advantage of requiring less data from each individual well, and thus may require less run time to collect data to use for calibration than required for training a separate model for each individual well. Another advantage of using a generalized model is that storing a single model may require less memory than required for storing separate models for each well of the protein sequencing device 502. Since each well may contain a single molecule, given the above approaches, a single model may be calibrated for a single molecule or for a number of molecules by considering multiple wells. According to some embodiments, calibration of a single model may be based on a number of molecules that is equal to or greater than 1, 10, 100, 1000, 10000, 100000, or 1000000. According to some embodiments, calibration of a single model may be based on a number of molecules that is less than or equal to 1000000, 100000, 10000, 1000, 100, 10 or 1. Any suitable combinations of the above-referenced ranges are also possible (e.g., a number of molecules that is equal to or greater than 1 and less than or equal to 10000).

Calibration may be performed at any suitable time. For example, calibration may be desirable prior to first using the protein sequencing device 502, upon using a new set of labels, upon a change in environmental conditions in which the protein sequencing device 502 is used, or after a period of use to account for aging of components of the protein sequencing device 502. The calibration may also be performed in response to a request from a user, such as by pressing a button on the instrument or sending a calibration command to the instrument from another device, or automatically based on a schedule or on an as-needed basis in response to a software command.

FIG. 5C illustrates an example well of the wells 502B part of the protein sequencing device 502. In the illustrated example of FIG. 5C, the well holds a sample 502F of a protein that is being sequenced, and reagents 502G that bind with amino acids of the sample 502F.

In some embodiments, the sample 502F of the protein may include one or more polypeptides of the protein. The polypeptide(s) may be immobilized to a surface of the well as illustrated in FIG. 5C. In some embodiments, the sample 502F data may be collected by the sensor(s) based on consecutive binding and cleavage interactions of one or more of the reagents 502G with a terminal amino acid of the sample 502F. In some embodiments, the reagents 502G may bind with amino acids of the sample 502F at substantially the same time. In some embodiments, multiple types of reagents may be engineered to bind with all or a subset of amino acids. The combination of one or more reagents that bind with an amino acid may result in detected values of properties of binding interactions (e.g., luminescence intensity, luminescence lifetime, pulse duration, inter-pulse duration, wavelength, and/or any value derived therefrom) that may be used for identifying the polypeptide. In some embodiments, the each of the combination of reagents (e.g., molecules) may have different properties. As an example, each of the reagents may have different binding affinities (K_{D}), rates of binding (kₒₙ), and/or off rate of binding (k_{off}). As another example, luminescent labels associated with reagents and/or amino acids may have different fluorescence properties. Examples of reagents and binding interactions of reagents with amino acids are described herein with reference to FIGS. 1-4.

In some embodiments, the reagents 502G may be tagged with luminescent labels. The reagents may be engineered to selectively bind to one or more amino acids as described above with reference to FIGS. 1-4. In some embodiments, one or more amino acids of the polypeptide 502F may be tagged with luminescent labels. As an example, one or more types of amino acids may be tagged with luminescent labels. The excitation source(s) 502A may apply excitation energy (e.g., light pulses) to the well as binding interactions occur between one or more of the reagents 502G and amino acids of the polypeptide 502F. The application of the excitation energy may result in light emissions by the luminescent labels that the reagents 502G and/or amino acids are tagged with. The light emissions may be detected by the sensor(s) 502C to generate data. The data may then be used to identify a polypeptide as described herein.

Although the example embodiment of FIGS. 5A-C describe use of binding interaction data obtained from detection of light emissions by luminescent labels, some embodiments may obtain binding interaction data using other techniques. In some embodiments, a protein sequencing device may be configured to access binding interaction data obtained from detection of electrical signals detected for binding interactions. For example, the protein sequencing device may include electrical signals that detect a voltage signal that is sensitive to binding interactions. The protein identification system 502D may be configured to use the voltage signal to determine pulse duration values and/or interpulse duration values. Some embodiments are not limited to a particular technique of detecting binding interactions of reagents with amino acids.

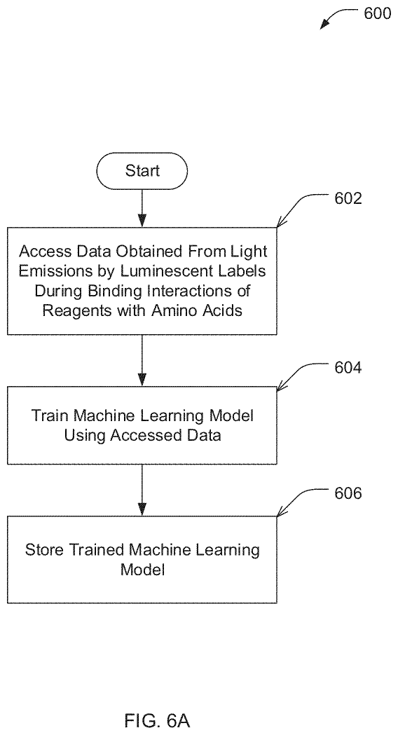

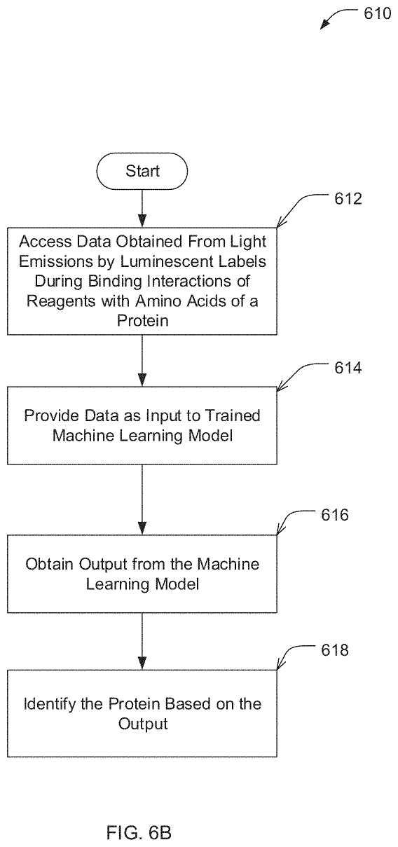

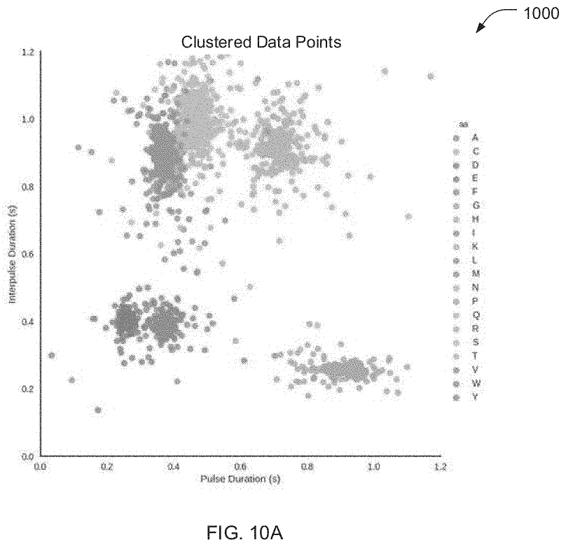

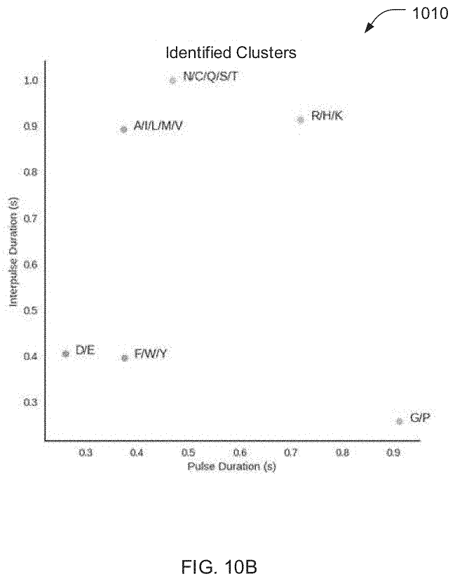

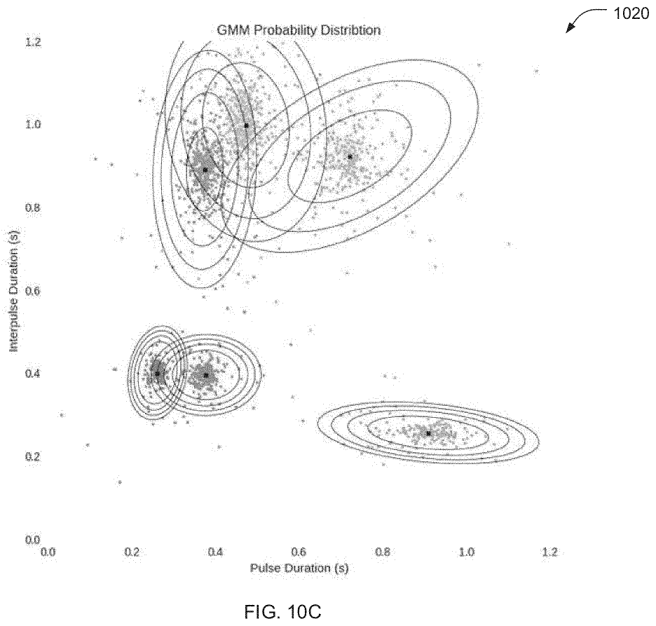

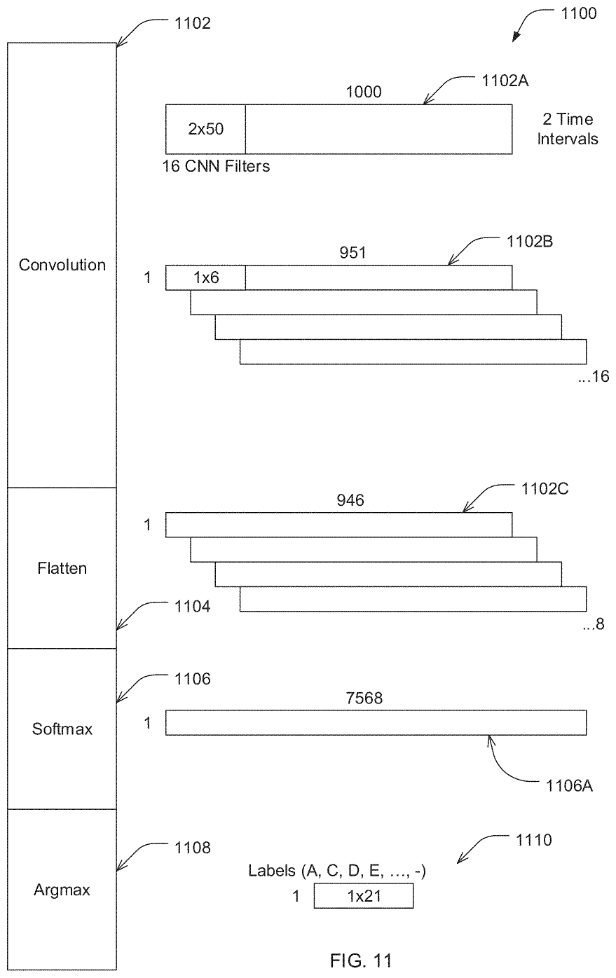

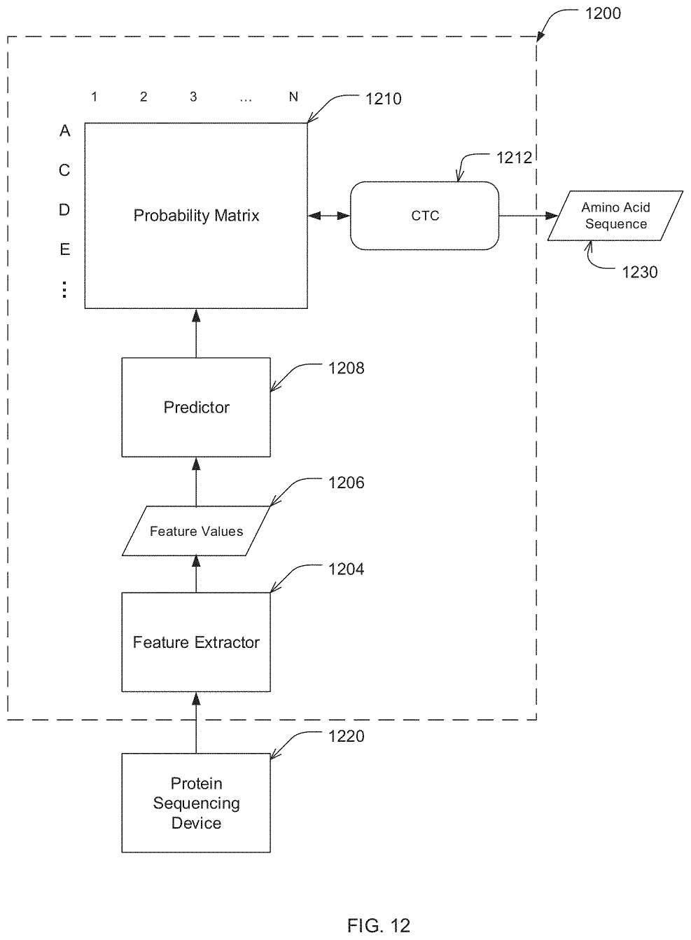

FIG. 6A illustrates an example process 600 for training a machine learning model for identifying a polypeptide, according to some embodiments of the technology described herein. Process 600 may be performed by any suitable computing device(s). As an example, process 600 may be performed by model training system 504 described with reference to FIG. 5A. Process 600 may be performed to train machine learning models described herein. As an example, process 600 may be performed to train a clustering model and/or a Gaussian mixture model (GMM) as described with reference to FIGS. 10A-C. As another example, the process 600 may be performed to train convolutional neural network (CNN) 1100 described with reference to FIG. 11. As another example, the process 600 may be performed to train a connectionist temporal classification (CTC)-fitted neural network model 1200 described with reference to FIG. 12.

In some embodiments, the machine learning model may be a clustering model. In some embodiments, each cluster of the model may be associated with one or more amino acids. As an illustrative example, the clustering model may include 5 clusters, where each cluster is associated with a respective set of amino acids. For example, the first cluster may be associated with alanine, isoleucine, leucine, methionine, and valine; the second cluster may be associated with the asparagine, cysteine, glutamine, serine, and threonine; the third cluster may be associated with arginine, histidine, and lysine; the fourth cluster may be associated with aspartic acid and glutamic acid; and the fifth cluster may be associated with phenylalanine, tryptophan, and tyrosine. Example numbers of clusters and associated amino acids are described herein for illustrative purposes. Some embodiments are not limited to any particular number of clusters or associations with particular sets of amino acids described herein.

In some embodiments, the machine learning model may be a deep learning model. In some embodiments, the deep learning model may be a neural network. As an example, the machine learning model may be a convolutional neural network (CNN) that generates an output identifying one or more amino acids of a polypeptide for a set of data provided as input to the CNN. As another example, the machine learning model may be a CTC-fitted neural network. In some embodiments, portions of the deep learning model may be trained separately. As an example, the deep learning model may have a first portion which encodes input data in values of one or more features, and a second portion which receives the values of the feature(s) as input to generate an output identifying one or more amino acids of the polypeptide.

In some embodiments, the machine learning model may include multiple groups (e.g., classes or clusters), and the machine learning model may include a separate model for each group. In some embodiments, the model for each group may be a mixture model. As an example, the model may include a Gaussian mixture model (GMM) for each of the groups for determining likelihoods that amino acids associated with the group are present at a location in the polypeptide. Each component distribution of a GMM for a respective group may represent amino acids associated with the respective group. As an example, the GMM for the first cluster described in the above example may include five component distributions: a first distribution for alanine, a second distribution for isoleucine, a third distribution for leucine, a fourth distribution for methionine, and a fifth distribution for threonine.

Process 600 begins at block 602, where the system executing process 600 accesses training data obtained from light emissions by luminescent labels during binding interactions of reagents with amino acids of a polypeptide. In some embodiments, the data may be collected by one or more sensors (e.g., sensor(s) 502C described with reference to FIG. 5B) for binding interactions of the reagents with amino acids in one or more wells of a protein sequencing device (e.g., device 502). In some embodiments, the light emissions may be emitted in response to one or more light pulses (e.g., laser pulses).

In some embodiments, the system may be configured to access the training data by determining values of one or more properties of binding interactions from data collected by the sensor(s). Examples of properties of binding interactions are described herein. In some embodiments, the system may be configured to use the one or more properties of the binding interactions as input features for the machine learning model. In some embodiments, the system may be configured to access the training data by accessing a number of photons detected in multiple time intervals of a time period after each of the light pulses. In some embodiments, the system may be configured to arrange the data in one or more data structures (e.g., a matrix, or an image), illustrative examples of which are described herein.

Next, process 600 proceeds to block 604 where the system trains a machine learning model using the training data accessed at block 602.

In some embodiments, the data accessed at block 602 may be unlabeled and the system may be configured to apply an unsupervised training algorithm to training data to train the machine learning model. In some embodiments, the machine learning model may be a clustering model and the system may be configured to identify clusters of the clustering model by applying an unsupervised learning algorithm to training data. Each cluster may be associated with one or more amino acids. As an example, the system may perform k-means clustering to identify clusters (e.g., cluster centroids) using the training data accessed at block 602.

In some embodiments, the system may be configured to perform supervised training. The system may be configured to train the model using information specifying one or more predetermined amino acids associated with the data accessed at block 602. In some embodiments, the system may be configured to train the machine learning model by: (1) providing the data accessed at block 602 as input to the machine learning model to obtain output identifying one or more amino acids; and (2) training the machine learning model based on a difference between the amino acid(s) identified by the output and predetermined amino acids. As an example, the system may be configured to update one or more parameters of the machine learning model based on the determined difference. In some embodiments, the information specifying one or more amino acids may be labels for the data obtained at block 602. In some embodiments, a portion of the data obtained at block 602 may be provided as input to the machine learning model and the output of the machine learning model corresponding to the portion of data may be compared to a label for the portion of data. In turn, one or more parameters of the machine learning model may be updated based on the difference between the output of the machine learning model and the label for the portion of data provided as input to the machine learning model. The difference may provide a measure of how well the machine learning model performs in reproducing the label when configured with its current set of parameters. As an example, the parameters of the machine learning model may be updated using stochastic gradient descent and/or any other iterative optimization technique suitable for training neural networks.

In some embodiments, the system may be configured to apply a semi-supervised learning algorithm to training data. The model training system 504 may (1) label a set of unlabeled training data by applying an unsupervised learning algorithm (e.g., clustering) to training data; and (2) applying a supervised learning algorithm to the labelled training data. As an example, the system may apply k-means clustering to the training data accessed at block 602 to cluster the data. The system may then label sets of data with a classification based on cluster membership. The system may then train the machine learning model by applying a stochastic gradient descent algorithm and/or any other iterative optimization technique to the labelled data.

In some embodiments, the machine learning model may classify data input into multiple groups (e.g., classes or clusters), where each group is associated with one or more amino acids. In some embodiments, the system may be configured to train a model for each group. In some embodiments, the system may be configured to train a mixture model for each group. The system may be configured to train a mixture model for a respective group by using training data obtained for binding interactions involving amino acid(s) associated with the respective group. As an example the system may train a Gaussian mixture model (GMM) for a respective group, for example, by using expectation minimization or any other suitable maximum likelihood or approximate maximum likelihood algorithm to identify parameters of component distributions of the GMM based on training data obtained for binding interactions involving amino acid(s) associated with the respective group.

After training the machine learning model at block 604, process 600 proceeds to block 606 where the system stores the trained machine learning model. The system may store value(s) of one or more trained parameters of the machine learning model. As an example, the machine learning model may include a clustering model with one or more centroids. The system may store identifications (e.g., coordinates) of the centroids. As another example, the machine learning model may include mixture models (e.g., GMMs) for groups of the machine learning model. The system may store parameters defining the component models. As another example, the machine learning model may include one or more neural networks. The system may store values of trained weights of the neural network(s). In some embodiments, the system may be configured to store the trained machine learning model for use in identifying polypeptides according to techniques described herein.

In some embodiments, the system may be configured to obtain new data to update the machine learning model using new training data. In some embodiments, the system may be configured to update the machine learning model by training a new machine learning model using the new training data. As an example, the system may train a new machine learning model using the new training data. In some embodiments, the system may be configured to update the machine learning model by retraining the machine learning model using the new training data to update one or more parameters of the machine learning model. As an example, the output(s) generated by the model and corresponding input data may be used as training data along with previously obtained training data. In some embodiments, the system may be configured to iteratively update the trained machine learning model using data and outputs identifying amino acids (e.g., obtained from performing process 610 described below in reference to FIG. 6B). As an example, the system may be configured to provide input data to a first trained machine learning model (e.g., a teacher model), and obtain an output identifying one or more amino acids. The system may then retrain the machine learning model using the input data and the corresponding output to obtain a second trained machine learning model (e.g., a student model).

In some embodiments, the system may be configured to train a separate machine learning model for each well of a protein sequencing device (e.g., protein sequencing device 502). A machine learning model may be trained for a respective well using data obtained from the well. The machine learning model may be tuned for characteristics of the well. In some embodiments, the system may be configured to train a generalized machine learning model that is to be used for identifying amino acids in multiple wells of a sequencer. The generalized machine learning model may be trained using data aggregated from multiple wells.

FIG. 6B illustrates an example process 610 for using a trained machine learning model obtained from process 600 for identifying a polypeptide, according to some embodiments of the technology described herein. Process 610 may be performed by any suitable computing device. As an example, process 610 may be performed by protein identification system 502D described above with reference to FIG. 5B.

Process 610 begins at block 612 where the system accesses data obtained from light emissions by luminescent labels from binding interactions of reagents with amino acids of a polypeptide. In some embodiments, the data may be obtained from data collected by one or more sensors (e.g., photodetector(s)) during amino acid sequencing performed by a protein sequencing device (e.g., device 502). As an example, the system may process data collected by the sensor(s) to generate the data.

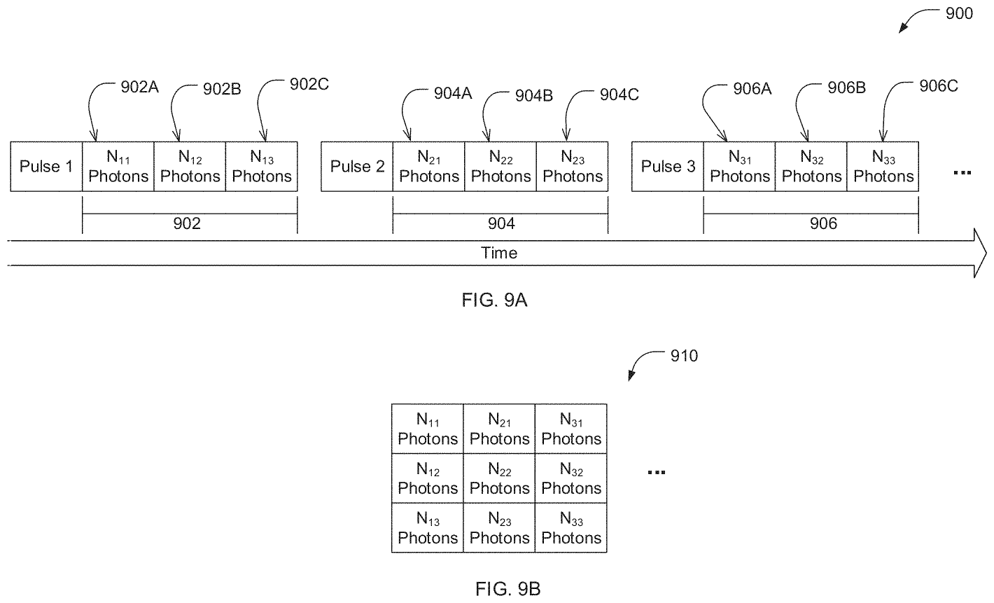

In some embodiments, the data may include values of one or more properties of binding interactions determined from data collected by the sensor(s) and values determined therefrom. Examples of properties and parameters determined therefrom are described herein. In some embodiments, the light emissions may be responsive to a series of light pulses. The data may include numbers of photons detected in one or more time intervals of time periods after the light pulses. As an example, the data may be data 900 described below with reference to FIG. 9A. In some embodiments, the system may be configured to arrange the data into a data structure 910 described below with reference to FIG. 9B.

In some embodiments, block 612 may comprise performing one or more signal processing operations on accessed data such as a signal trace. The signal processing operations may for instance include one or more filtering and/or subsampling operations, which may remove observed pulses within the data that are due to noise.

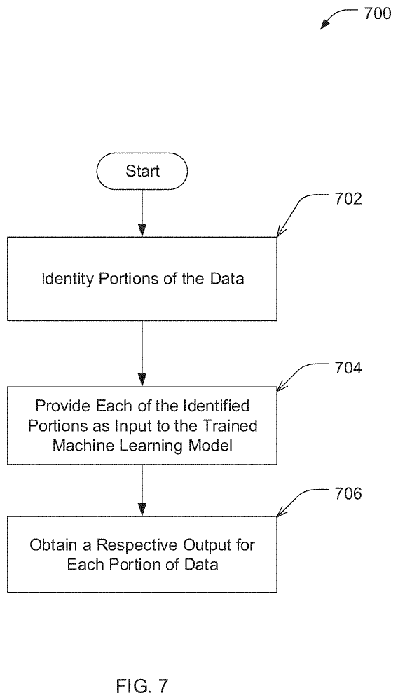

Next, process 600 proceeds to block 614 where the system provides the data accessed at block 606 as input to the trained machine learning model. In some embodiments, the system may be configured to provide the data as input, and obtain an output identifying amino acids of the polypeptide. As an example, the system may provide the data obtained at block 612 as input to a CTC-fitted neural network model, and obtain an output (e.g., a sequence of letters) identifying an amino acid sequence of the polypeptide. In some embodiments, the system may be configured to divide the data into multiple portions and provide the data for each of the portions as a separate input to the trained machine learning model to obtain a corresponding output (e.g., as described below with reference to FIG. 7). As an example, the system may identify portions of data associated with respective binding interactions of a reagent with an amino acid of the polypeptide.

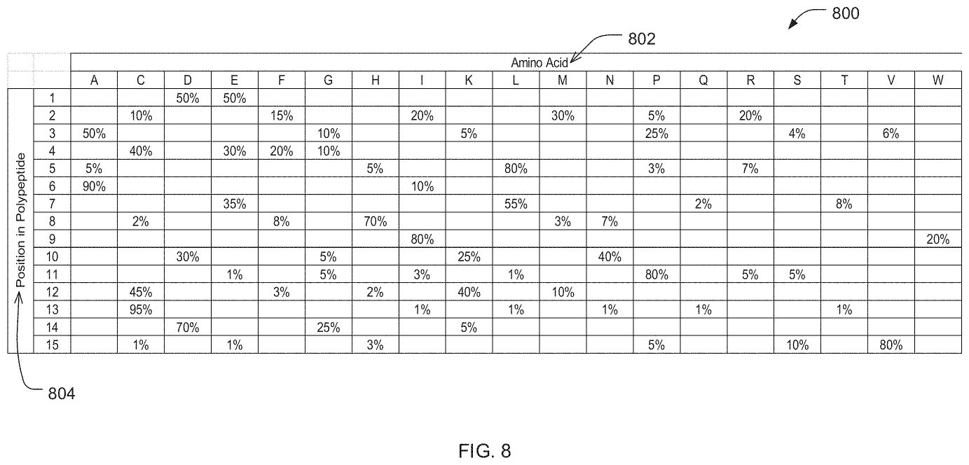

Next, process 600 proceeds to block 616 where the system obtains an output from the machine learning model. In some embodiments, the system may be configured to obtain an output indicating, for each of multiple locations in the polypeptide, one or more likelihoods that one or more respective amino acids is present at the location in the polypeptide. As an example, the output may indicate, for each location, likelihoods that each of twenty amino acids is present at the location. An example depiction of output obtained from the machine learning system is described below with reference to FIG. 8.

In some embodiments, the system may be configured to obtain an output for each of multiple portions of data provided to the machine learning model. An output for a respective portion of data may indicate an amino acid at a particular location in the polypeptide. In some embodiments, the output may indicate likelihoods that one or more respective amino acids are present at a location in the polypeptide associated with the portion of data. As an example, an output corresponding to a portion of data provided as input to the machine learning model may be a probability distribution specifying, for each of multiple amino acids, a probability that the amino acid is present at a respective location in the polypeptide.

In some embodiments, the system may be configured to identify an amino acid that is present at a location in the polypeptide associated with the portion of data. As an example, the system may determine a classification specifying an amino acid based on the output for data provided to the machine learning model. In some embodiments, the system may be configured to identify an amino acid based on likelihoods that respective amino acid(s) are present at a location in the polypeptide. As an example, the system may identify the amino acid to be the one of the respective amino acid(s) that has the greatest likelihood of being present at the location in the polypeptide. In some embodiments, the system may be configured to identify the amino acid based on value(s) of one or more properties of binding interactions and/or other parameters without using the machine learning model. As an example, the system may determine that a pulse duration and/or inter-pulse duration for the portion of data is associated with a reagent that selectively binds to a particular type of protein, and identify the amino acid that is present at the location to be an amino acid of that type.

In some embodiments, the system may be configured to obtain a single output identifying amino acids of the polypeptide. As an example, the system may receive a sequence of letters identifying the amino acids of the polypeptide. As another example, the system may receive a series of values for each of multiple locations in the polypeptide. Each value in a series may indicate a likelihood that a respective amino acid is present at a respective location in the polypeptide.

In some embodiments, the system may be configured to normalize output obtained from the machine learning model. In some embodiments, the system may be configured to receive a series of values from the machine learning model, where each value indicates a likelihood that a respective amino acid is present at a respective location in the polypeptide. The system may be configured to normalize the series of values. In some embodiments, the system may be configured to normalize the series of values by applying a softmax function to obtain a set of probability values that sum to 1. As an example, the system may receive a series of output values from a neural network, and apply a softmax function to the values to obtain a set of probability values that sum to 1. In some embodiments, the system may be configured to receive outputs from multiple models (e.g., GMMs), where each model is associated with a respective set of amino acids. The output from each model may be values indicating, for each of a set of amino acids associated with the model, a likelihood that the amino acid is present at a location in the polypeptide. The system may be configured to normalize the values received from all the multiple models to obtain the output. As an example, the system may (1) receive a first set of probability values for a first set of amino acids from a first GMM, and probability values for a second set of amino acids from a second GMM; and (2) apply a softmax function to the joint first and second sets of probability values to obtain a normalized output. In this example, the normalized output may indicate, for each amino acid in the first and second sets of amino acids, a probability that the amino acid is present at a location in the polypeptide, where the probability values sum to 1.

After obtaining the output from the trained machine learning model at block 616, process 610 proceeds to block 618 where the system identifies the polypeptide using the output obtained from the machine learning model. In some embodiments, the system may be configured to match the output obtained at block 616 to one of a known set of amino acid sequences and associated proteins stored in a data store (e.g., accessible by protein sequencing device 502). The system may identify the polypeptide to be a part of the protein associated with the amino acid sequence that the output is matched to. As an example, the data store may be a database of amino acid sequences from the human genome (e.g., UniProt and/or the HPP databases).

In some embodiments, the system may be configured to match the output to an amino acid sequence by (1) generating a hidden Markov model (HMM) based on the output; and (2) using the HMM to identify an amino acid sequence that the data most closely aligns to from amongst multiple amino acid sequences. In some embodiments, the output may indicate, for each of a plurality of locations in the polypeptide, likelihoods that respective amino acids are present at the location. An example depiction of output from the machine learning model is described below with reference to FIG. 8. The system may be configured to use the output to determine values of parameters of the HMM. As an example, each state of the HMM may represent a location in the polypeptide. The HMM may include probabilities of amino acids being at different locations. In some embodiments, the HMM may include insertion and deletion rates. In some embodiments, the insertions and deletion rates may be preconfigured values. In the HMM. In some embodiments, the system may be configured to determine the values of the insertion and deletion rates based on the output obtained from the machine learning model at block 616. In some embodiments, the system may be configured to determine the insertion and deletion rates based results of one or more previous polypeptide identification processes. As an example, the system may determine the insertion and deletion rates based on one or more previous polypeptide identifications and/or outputs of the machine learning model obtained from performing process 610.

In some embodiments, the system may be configured to identify the polypeptide using the output obtained from the machine learning model by (1) determining a sequence of amino acids based on the output obtained from the machine learning model; and (2) identifying the polypeptide based on the sequence of amino acids. The determined sequence of amino acids may be a portion (e.g., a peptide) of the polypeptide. In some embodiments, the output may indicate, for each of multiple locations in the polypeptide, likelihoods that respective amino acids are present at the location. The system may be configured to determine the sequence of amino acids by (1) identifying, for each of the locations, one of the respective amino acids that has the greatest likelihood of being present at the location; and (2) determining the sequence of amino acids to be the set of amino acids identified for the locations. As an example, the system may determine that, of a possible twenty amino acids, alanine (A) has a maximum likelihood of being present at a first location in the polypeptide, glutamic acid (E) has a maximum likelihood of being present at a second location in the polypeptide, and that aspartic acid (D) has a maximum likelihood of being present at a third location. In this example, the system may determine at least a portion of a sequence of amino acids to be alanine (A), glutamic acid (E), and aspartic acid (D). In some embodiments, the system may be configured to identify the polypeptide based on the determined sequence of amino acids by matching the amino acid sequence to one from a set of amino acid sequences specifying proteins. As an example, the system may match the determined sequence of amino acids to a sequence from the Uniprot and/or HPP databases, and identify the polypeptide to be part of the protein associated with the matched sequence.

In some embodiments, the system may identify the polypeptide using the output obtained from the machine learning model in block 618 by matching the determined sequence of amino acids to a pre-selected panel. In contrast to the approach in which the system matches the determined sequence of amino acids to a sequence from a database of known polypeptides, in some cases the system may match the sequence to a pre-selected panel that may for instance be a subset of such a database. For example, the polypeptide may be one of a set of polypeptides with known clinical significance, and consequently it may be more accurate and/or more efficient to match the determined sequence of amino acids to one of the set of polypeptides rather than search an entire database containing all possible polypeptides. In some embodiments, the data input to the machine learning model may be generated by measuring light emission from an affinity reagent interacting with a polypeptide that is known to be one of the pre-selected panel of polypeptides. That is, the experimental procedure to generate the data may ensure that the polypeptide used to generate the data is one of the set of polypeptides being considered for matching by the machine learning model.

In some embodiments, the system may produce a list of relative probabilities for a plurality of polypeptides using the output obtained from the machine learning model in block 618. Rather than identifying a particular polypeptide as described above, it may be preferable to produce a list of several polypeptides along with the probabilities of each being the correct match. In some embodiments, confidence scores relating to aspects of the data may be generated based on such probabilities, such as a confidence score that a particular protein is present in a sample, and/or that a particular protein comprises at least some threshold fraction of the sample.

In some embodiments, the system may identify a variant of a polypeptide using the output obtained from the machine learning model in block 618. In particular, in some cases the system may determine that the most likely sequence is a variant of a reference sequence (e.g., a sequence in a database). Such variants may include naturally occurring or natural variants of a polypeptide, and/or a polypeptide in which an amino acid has been modified (e.g., phosphorylated). As such, in block 618 variants of a plurality of reference sequences may be considered to match the output from the machine learning model in addition to consideration of the reference sequences themselves.

FIG. 7 illustrates an example process 700 for providing input to a machine learning model, according to some embodiments of the technology described herein. Process 700 may be performed by any suitable computing device. As an example, process 700 may be performed by protein identification system 502D described above with reference to FIG. 5B. Process 700 may be performed as part of block 616 of process 610 described above with reference to FIG. 6B.

Prior to performing process 700, the system performing process 700 may access data obtained from detected light emissions by luminescent labels from binding interactions of reagents with amino acids. As an example, the system may access data as performed at block 612 of process 610 described above with reference to FIG. 6B.

Process 700 begins at block 702, where the system identifies portions of the data, also referred to herein as regions of interest (ROIs). In some embodiments, the system may be configured to identify portions of data corresponding to respective binding interactions. As an example, each identified portion of data may include data from a respective binding interaction of a reagent with an amino acid of a polypeptide. In some embodiments, the system may be configured to identify the portions of the data by identifying data points corresponding to cleavage of amino acids from a polypeptide. As discussed above with reference to FIGS. 1-3, a protein sequencing device may sequence a sample by iteratively detecting and cleaving amino acids from a terminal end of a polypeptide (e.g., polypeptide 502F shown in FIG. 5C). In some embodiments, cleaving may be performed by a cleaving reagent tagged with a respective luminescent label. The system may be configured to identify the portions of the data by identifying data points corresponding to light emissions by the luminescent label that the cleaving reagent is tagged with. As an example, the system may identify one or more luminescence intensities, luminescence lifetime values, pulse duration values, inter-pulse duration values, and/or photon bin counts. The system may then segment the data into portions based on the identified data points. In some embodiments, cleaving may be performed by an untagged cleaving reagent. The system may be configured to identify the portions of the data by identifying data points corresponding to periods of cleaving. The system may then segment the data into portions based on the identified data points.

In some embodiments, the system may be configured to identify the portions of data by identifying time intervals between time periods of light emissions. As an example, the system may identify a time interval between two periods of time during which light pulses are emitted. The system may be configured to identify portions of data corresponding to respective binding interactions based on the identified time intervals. As an example, the system may identify a boundary between consecutive binding interactions by determining whether a duration of a time interval between light emission (e.g., light pulses) exceeds a threshold duration of time. The system may segment the data into portions based on boundaries determined from the identified time intervals.

In some embodiments, the system may be configured to identify portions of the data corresponding to respective binding interactions by (1) tracking a summary statistic in the data; and (2) identifying portions of the data based on points at which the summary statistic deviates. In some embodiments, the data may be time series data wherein each point represents values of one or more parameters taken at a particular point in time. The system may be configured to: (1) track the summary statistic in the data with respect to time; (2) identify data points at which the summary statistic deviates by a threshold amount; and (3) identify the portions of data based on the identified points. As an example, the system may track a moving mean pulse duration value relative to time in the data. The system may identify one or more points corresponding to a reaction with a binding interaction based on points at which the mean pulse duration value increases by a threshold amount. As another example, the system may track a moving mean luminescence intensity value relative to time in the data. The system may identify one or more points corresponding to a binding interaction based on points at which the mean luminescence intensity value increases by a threshold amount.

In some embodiments, the system may be configured to identify portions of the data by dividing the data into equally sized portions. In some embodiments, the data may include multiple frames, where each frame includes numbers of photons detected in each of one or more time intervals in a time period after application of an excitation pulse. The system may be configured to identify portions of the data by dividing the data into portions of equally sized frames. As an example, the system may divide the data into 1000, 5000, 10,000, 50,000, 100,000, 1,000,000 and/or any suitable number between 1000 and 1,000,000 frame portions. In some embodiments, the system may be configured to divide the data into frames based on determining a transition between two binding interactions. As an example, the system may identify values of photon counts in the bins that indicate a transition between two binding interactions. The system may allocate frames to portions based on the identified transitions in the data. In some embodiments, the system may be configured to reduce the size of each portion. As an example, the system may determine one or more summary statistics for strides (e.g., every 10 or 100 frames) of the portion of data.

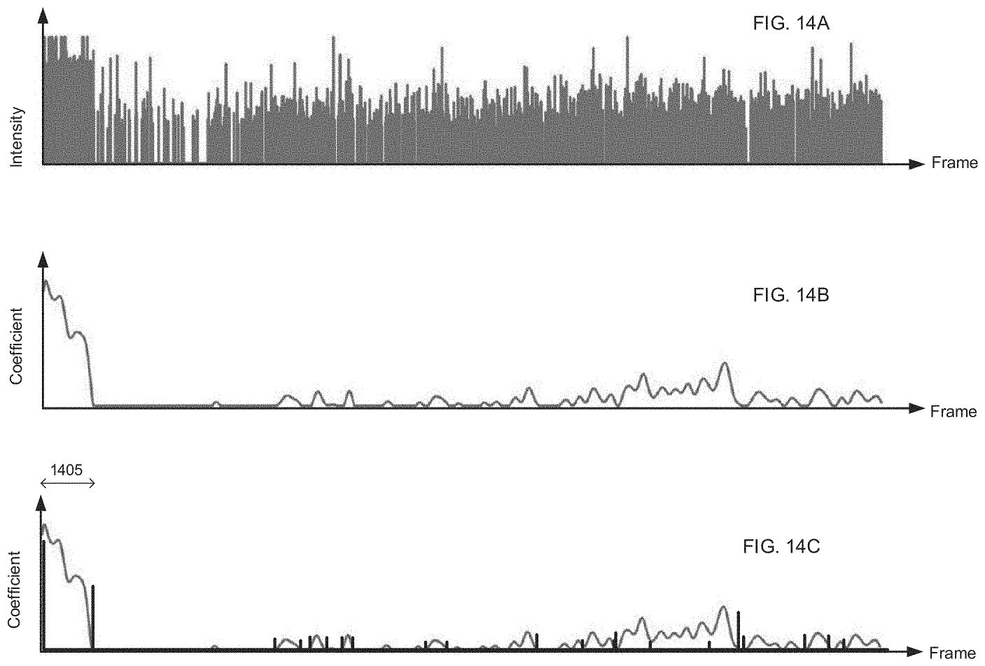

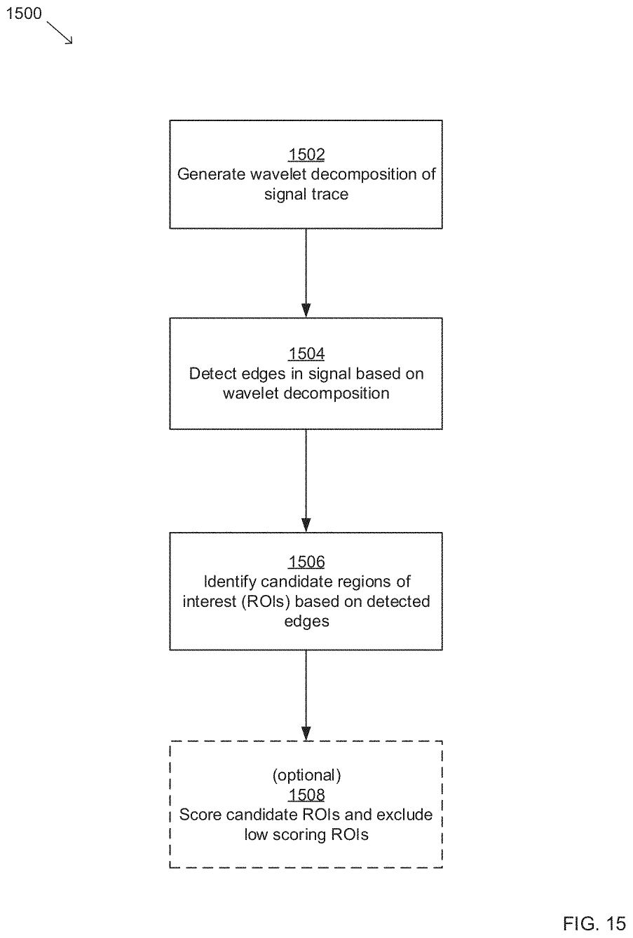

In some embodiments, the system may be configured to identify portions of the data by performing a wavelet transformation of the signal trace and identifying leading and/or falling edges of portions of the signal based on wavelet coefficients produced from the wavelet transformation. This process is discussed in greater detail below in relation to FIGS. 14A-14C and FIG. 15.

In some embodiments, the time intervals that are part of a time period are non-overlapping. In other embodiments, the time intervals that are part of a time period may overlap one another. Photon counts in an overlapping region of two time intervals may be added to the photon count for both time intervals. Data in overlapping time intervals may be statistically dependent on data in a neighboring time interval. In some embodiments, such a dependency may be used to process data (e.g., training data). As an example, the statistical dependency may be used to regularize and/or smooth the data.

After identifying portions of the data at block 702, process 700 proceeds to block 704 where the system provides input to a machine learning model based on the identified portions. In some embodiments, the system may be configured to determine values of one or more properties of detected binding interactions. These values may include any number of pulse parameters such as, but not limited to, pulse duration, inter-pulse duration, wavelength, luminescence intensity, luminescence lifetime values, pulse count per unit time, or combinations thereof. These values may be represent as a mean, medium, mode or by providing a plurality of measured pulse parameters for a given portion of the data. For instance, the input to the machine learning model in block 704 may comprise a mean pulse duration for an identified portion of the data.

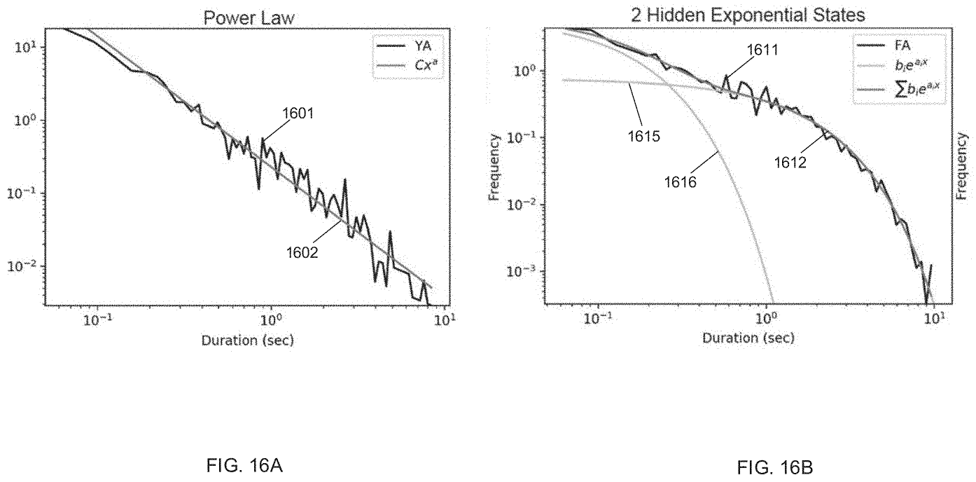

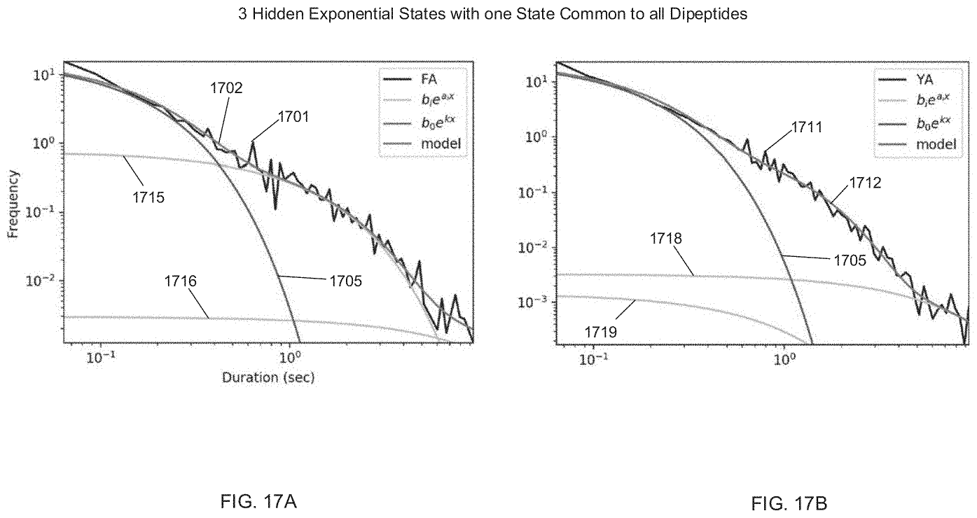

In some embodiments, values for input to the machine learning model may include any parameters derived from a portion of data identified in block 702. Parameters so derived may for instance include fitting suitable functions and/or distributions to measurements to pulse parameters. For example, the range of different pulse durations measured for a portion of the data identified in block 702 may be fit to an exponential function, a Gaussian distribution, a Poisson distribution, and the values describing those functions or distributions may be input to the machine learning model in block 704. As such, the values may for instance include a mean and variance of a Gaussian distribution that characterizes a number of different pulses observed with a portion of data identified in block 702. An example of fitting a plurality of exponential functions to a pulse parameter is described further below in relation to FIGS. 16A-16B and 17A-17B.

Irrespective of how the values are calculated in block 704, these values may also be provided as input to the machine learning model in block 704. The determined values may form a feature set of the respective binding interaction that is input to the machine learning model. In some cases, the portion of data may correspond to one or more frames and the determined values may form a feature set for the frame(s).

In some embodiments, the system may be configured to provide each identified portion of data as input to the machine learning model without determining values of properties of binding interactions and/or values of parameters determined from the properties. As an example, the system may provide each set of frames (e.g., each including one or more bin counts) that the data was divided into as input to the machine learning model.

Next, process 700 proceeds to block 706 where the system obtains an output corresponding to each portion of data input into the trained machine learning model. In some embodiments, each output may correspond to a respective location in the polypeptide. As an example, the output may correspond to a location in a polypeptide of a protein. In some embodiments, each output may indicate likelihoods of one or more amino acids being at the location in the polypeptide. As an illustrative example, each of the rows in the depiction 800 of the output of the machine learning system illustrated in FIG. 8 may be an output of the machine learning model corresponding to one of the identified portions of data. In some embodiments, each output may identify an amino acid involved in a respective binding interaction corresponding to the portion of data input into the machine learning model. In some embodiments, the system may be configured to use the outputs obtained at block 706 to identify a polypeptide. As an example, the system may use the outputs to identify a polypeptide as performed at block 618 of process 610 described above with reference to FIG. 6B.

FIG. 8 shows a table 800 depicting output obtained from a machine learning model, according to some embodiments of the technology described herein. As an example, the output depicted in FIG. 8 may be obtained at block 616 of process 610 described above with reference to FIG. 6B.

In the example table 800 of FIG. 8, the output obtained from the machine learning system includes, for each of multiple locations 804 in a polypeptide (e.g., of a protein), probabilities that respective amino acids 802 are present at the location. In the example depiction 800 of FIG. 8, the output includes probabilities for twenty amino acids. Each column of table 800 corresponds to a respective one of the twenty amino acids. Each amino acid is labelled with its respective single letter abbreviation in FIG. 8 (e.g., A, C, D, E, F, G, H, I, K, L, M, N, P, Q, R, S, T, V, W). Each row of table 800 specifies probabilities that each of the twenty amino acids is present at one of the locations in the polypeptide. As one example, for the location indexed by the number 1, the output indicates that there is a 50% probability that aspartic acid (D) is present at the location and a 50% probability that glutamic acid (E) is present at the location. As another example, for the location indexed by the number 10, the output indicates that there is a 30% probability that glutamic acid (D) is present at the location, a 5% probability that glycine (G) is present at the location, a 25% probability that lysine (K) is present at the location, and a 40% probability that asparagine (N) is present at the location.

Although the example embodiment of FIG. 8 shows likelihoods for twenty amino acids at 15 locations in a polypeptide, some embodiments are not limited to any number of positions or amino acids. Some embodiments may include likelihoods for any number of locations in a polypeptide, as aspects of the technology described herein are not limited in this respect. Some embodiments may include likelihoods for any number of amino acids, as aspects of the technology described herein are not limited in this respect.

FIG. 9A illustrates an example of data 900 that may be obtained from light emissions by luminescent labels, in accordance with some embodiments of the technology described herein. As an example, the data 900 may be obtained by the sensor(s) 502C of protein sequencing device 502 described above with reference to FIGS. 5A-C.

The data 900 indicates a number of photons detected in each of multiple time intervals after an excitation light pulse. A number of photons may also be referred to herein as a “photon count.” In the example illustrated in FIG. 9A, the data 900 includes numbers of photons detected during time intervals after three pulses of excitation light. In the example illustrated in FIG. 9A, the data 900 includes: (1) a number of photons detected in a first time interval 902A, a second time interval 902B, and a third time interval 902C of a time period 902 after the first excitation light pulse; (2) a number of photons detected in a first time interval 904A, a second time interval 904B, and a third time interval 904C of a time period 904 after the second excitation light pulse; and (3) a number of photons detected in a first time interval 906A, a second time interval 906B, and a third time interval 906C of a time period 906 after the third excitation light pulse.

In some embodiments, each of the time intervals in a period of time after a pulse of excitation light may be of equal or substantially equal duration. In some embodiments, the time intervals in the period of time after a pulse of excitation light may have varying duration. In some embodiments, the data may include numbers of photons detected in a fixed number of time intervals after each pulse of excitation light. Although the data includes three time intervals in each time period following a pulse of excitation light, the data may be binned into any suitable number of time intervals, as aspects of the technology described herein are not limited in this respect. Also, although the example of FIG. 9A shows data for three time periods following three pulses of excitation light, the data 900 may include data collected during time periods after any suitable number of excitation light pulses, as aspects of the technology described herein are not limited in this respect. Also, although the example of FIG. 9A shows that the intervals of a time period are disjointed, in some embodiments the intervals may overlap.

FIG. 9B illustrates an example arrangement of the data 900 from FIG. 9A which may be provided as input to a machine learning model, according to some embodiments of the technology described herein. As an example, the data structure 910 may be generated as input to a deep learning model (e.g., a neural network) to obtain an output identifying amino acids.

As illustrated in FIG. 9B, the numbers of photons from the data 900 may be arranged into a data structure 910 that includes multiple series of values. In some embodiments, the data structure 910 may be a two-dimensional data structure encoding a matrix (e.g., an array, a set of linked lists, etc.). Each of the series of values may form a row or column of the matrix. As may be appreciated, the data structure 910 may be considered as storing values of an image, where each “pixel” of the image corresponds to a respective time interval in a particular time period after a corresponding excitation light pulse and the value of the pixel indicates the number of photons detected during the time interval.

In the example illustrated in FIG. 9B, the data structure 910 includes multiple series of data in columns. Each column may also be referred to herein as a “frame.” The data structure 910 includes: (1) a first frame that specifies the numbers of photons N₁₁, N₁₂, N₁₃ detected in the time intervals 902A-C of the time period 902 after the first pulse of excitation light; (2) a second frame that specifies the numbers of photons N₂₁, N₂₂, N₂₃ detected in the time intervals 904A-C of the time period 904 after the second pulse of excitation light; and (3) a third frame that specifies the numbers of photons N₃₁, N₃₂, N₃₃ detected in the time intervals 906A-C of the time period 906 after the third pulse of excitation light. Although the example illustrated in FIG. 9B shows three frames, the data structure 910 may hold data from any suitable number of frames, as aspects of the technology described herein are not limited in this respect.

In the example illustrated in FIG. 9B, the data structure 910 includes multiple series of data in rows. Each row specifies numbers of photons detected in a particular bin for each pulse of excitation light. The data structure 910 includes a first series of values that includes: (1) the number of photons N₁₁ in the first interval 902A in the time period 902 after the first pulse of excitation light; (2) the number of photons N₂₁ in the first interval 904A in the time period 904 after the second pulse of excitation light; and (3) the number of photons N₃₁ in the first interval 906A in the time period 906 after the third pulse of excitation light. The data structure 910 includes a second series of values that includes: (1) the number of photons N₁₂ in the second interval 902B in the time period 902 after the first pulse of excitation light; (2) the number of photons N₂₂ in the second interval 904B in the time period 904 after the second pulse of excitation light; and (3) the number of photons N₃₂ in the second interval 906B in the time period 906 after the third pulse of excitation light. The data structure 910 includes a third series of values that includes: (1) the number of photons N₁₃ in the third interval 902C in the time period 902 after the first pulse of excitation light; (2) the number of photons N₂₃ in the third interval 904C in the time period 904 after the second pulse of excitation light; and (3) the number of photons N₃₃ in the third interval 906C in the time period 906 after the third pulse of excitation light.

FIGS. 10A-C illustrate steps for training a machine learning system, according to some embodiments of the technology described herein. As an example, FIGS. 10A-C illustrate various steps of training a machine learning model that may be performed as part of process 600 described above with reference to FIG. 6A by model training system 504 described above with reference to FIG. 5A.

FIG. 10A shows a plot 1000 of clustering of data accessed from detected light emissions by luminescence labels from binding interactions of reagents with amino acids. In the example of FIG. 10A, the plot 1000 shows results of clustering of data among six clusters. In some embodiments, the system (e.g., model training system 504) may be configured to cluster the data points to identify clusters (e.g., centroids and/or boundaries between clusters). In some embodiments, the clustering may be performed as part of process 600, described in reference to FIG. 6A, to train a clustering model. As an example, the system may apply an iterative algorithm (e.g., k-means) to the data points to obtain the clustering result shown in the example of FIG. 10 A.

In some embodiments, data clusters may be identified by sequencing a known peptide having a known sequence of amino acids and generating data (e.g., pulse duration and interpulse duration data) corresponding to each of the known amino acids. This process may be repeated numerous times to produce an understanding of where data for particular known amino acids will cluster with respect to the various pulse characteristics being evaluated.

FIG. 10B shows a plot 1010 of clusters (e.g., coordinates of cluster centroids) identified from the clustered points shown in plot 1000 of FIG. 10A. As an example, each of the centroids shown in plot 1010 may be determined to be a mean pulse duration and inter-pulse duration value of the data points in a respective cluster. In the example of FIG. 10A, each centroid is associated with a different set of amino acids. Plot 1010 shows (1) a first centroid associated with amino acids A, I, L, M, and V; (2) a second centroid associated with amino acids N, C, Q, S, and T; (3) a third centroid associated with amino acids R, H, and K; (4) a fourth centroid associated with amino acids D and E; (5) a fifth centroid associated with F, W, and Y; and (6) a sixth centroid associated with amino acids G and P.

FIG. 10C shows a plot 1020 of a result of training a Gaussian mixture model (GMM) for each of the clusters shown in plots 1000 and 1010. Each concentric circle shown in plot 1020 marks boundaries of equivalent probabilities. In some embodiments, each component of a GMM model trained for a respective cluster represents an amino acid associated with the respective cluster. The clustering model, with a GMM model trained for each cluster, may then be used for identifying a polypeptide as described above with reference to FIG. 6B. As an example, data accessed from detected light emissions by luminescent labels from binding interactions of reagents with amino acids of an unknown polypeptide may be input into the model. In some embodiments, each input to the machine learning model may correspond to a respective binding interaction of a reagent with an amino acid at a respective location in the polypeptide. A portion of data may be classified into one of the clusters shown in plot 1020, and the GMM trained for the cluster may be used to determine likelihoods that one or more amino acids associated with the cluster are at the location in the polypeptide. In some embodiments, the system may be configured to normalize likelihoods obtained from the GMMs in a joint probability space. As an example, the system may apply a softmax function to likelihoods obtained from the GMMs to obtain a probability value for each of multiple amino acids, where the probability values sum to 1.

As an alternative to training a GMM for each of the clusters as shown in FIG. 10C, in some embodiments a single GMM may be fit to a mixture of Gaussians for all of the clusters. In some cases, such a fit may be based on characteristics of the identified clusters such as the number of clusters and where their centroids are located. Alternatively, if labels are known for each of the data points, the parameters of a single GMM may be directly initialized using the measured variances and centroids of each cluster.

Although the examples of FIGS. 10A-C describe use of a GMM model for each cluster, some embodiments may use another type of model, as embodiments are not limited in this respect. As an example, a support vector machine (SVM) may be trained for each of the clusters (or a single SVM may be trained for all of the clusters together) and used to classify a portion of data as one of multiple amino acids associated with the cluster. As another example, a neural network may be trained for each of the clusters (or a single neural network may be trained for all of the clusters together) and used to obtain likelihoods that each of the amino acids associated with the cluster is present at a location in the polypeptide.

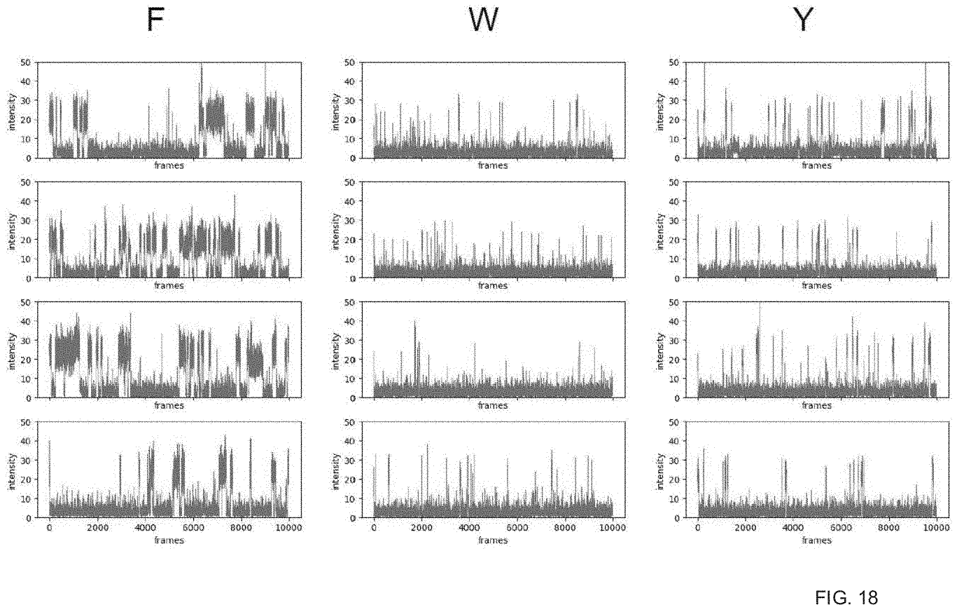

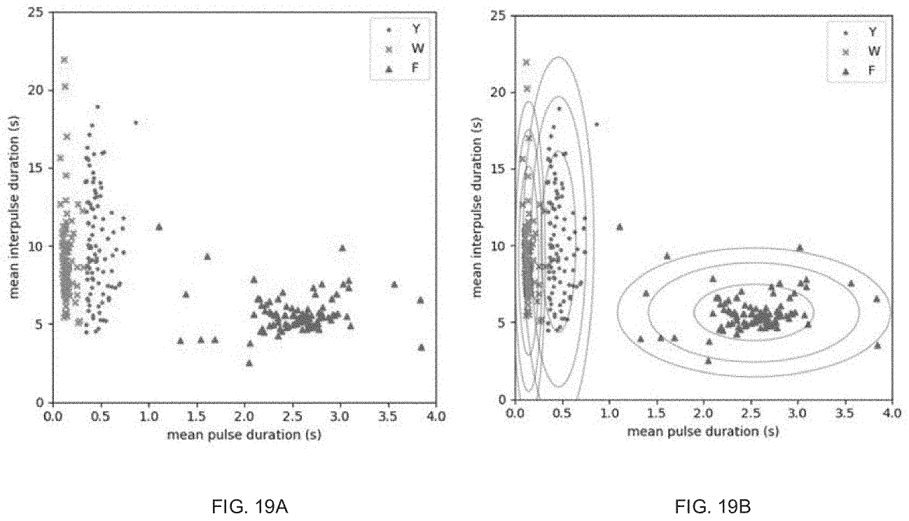

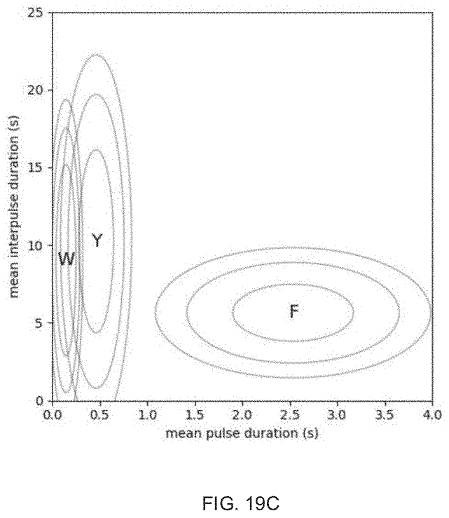

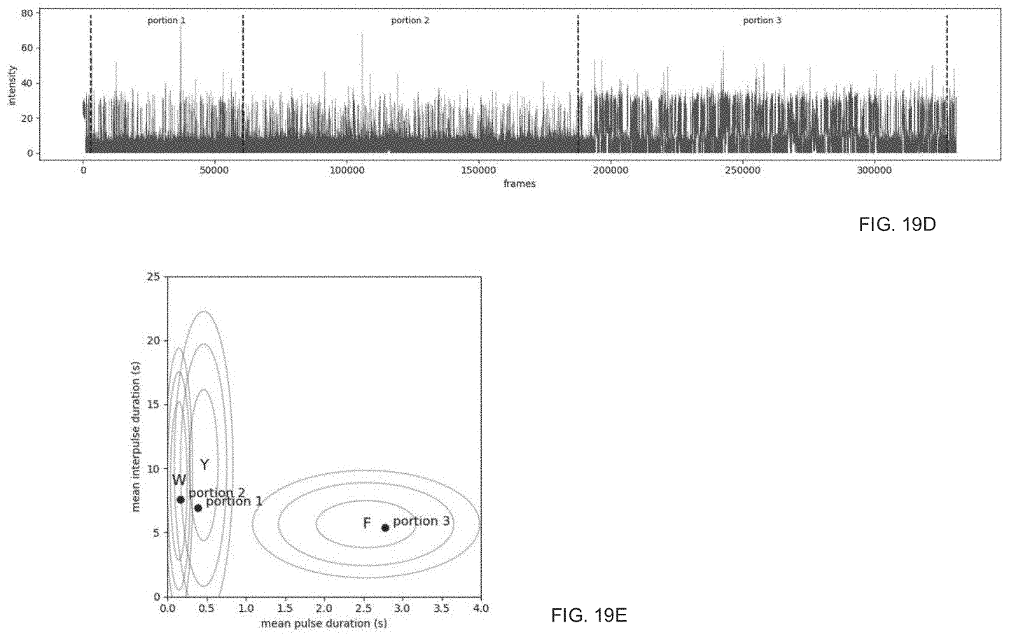

The above-described process of training a machine learning model using a GMM model, and utilizing the machine learning model to identify one or more amino acids, is further illustrated by FIGS. 18 and 19A-19E. FIG. 18 depicts a number of signal traces representing data obtained by measuring light emissions from a sample well as described above. In the example of FIG. 18, signal traces shown were produced by interaction of an affinity reagent with three different amino acid residues in the N-terminal position of a peptide: the first column of four signal traces are known to have been produced by interaction with the “F” amino acid, the second column by the “W” amino acid, and the third column by the “Y” amino acid. As a result, these signal traces may be used to train a machine learning model as described above in relation to FIG. 6. In general, many more signal traces than the few shown in FIG. 18 may be used as input to train the machine learning model.

FIGS. 19A-19E depict a process of training a GMM-based machine learning model based on signal traces for three amino acids such as those shown in FIG. 18. FIG. 19A depicts data obtained from signal traces that were produced from interaction of an affinity reagent with known amino acids, either F, W or Y, according to some embodiments. In particular, the data shown in FIG. 19A depicts characteristics of pulses from the signal traces, with the mean characteristics of pulses for each signal trace being represented by a data point. A data point for the Y amino acid (dark circles), for example, represents the mean pulse duration and mean interpulse duration for the pulses in a signal trace known to have been produced from reactions with the Y amino acid.

As shown in FIG. 19B, and as discussed above, a GMM may be generated for such data by identifying clusters corresponding to each dataset corresponding to a known amino acid. These three clusters are shown in FIG. 19B for the data shown in FIG. 19A, and are shown without these data points in FIG. 19C.

Once trained, a machine learning model that includes the GMM represented by FIGS. 19B and 19C may be applied to unlabeled data such as that shown in FIG. 19D. In the example of FIG. 19D, a signal trace is depicted that contains data that may have been produced from a number of different amino acids (or from affinity reagents associated therewith). As discussed above in relation to FIG. 7, portions of the data may be identified based on pulse characteristics or otherwise to identify portions that may have been produced through different interactions. Each of these portions (or characteristics thereof) may be input to the trained machine learning model to determine which amino acid is associated with each portion. As shown in FIG. 19E, this may result in a position in the two-dimensional space defined by mean pulse duration and mean interpulse duration being determined for each portion. An amino acid most likely to be associated with each position in the space can thereby be determined based on the trained machine learning model. For example, as shown in FIG. 19E, portion 3 may be determined to be highly likely to be associated with the F amino acid.

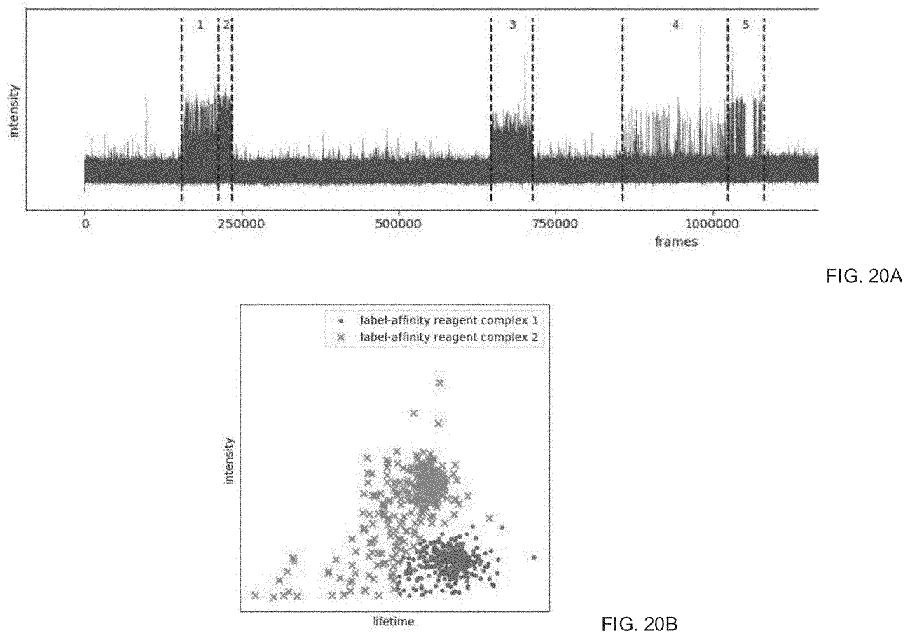

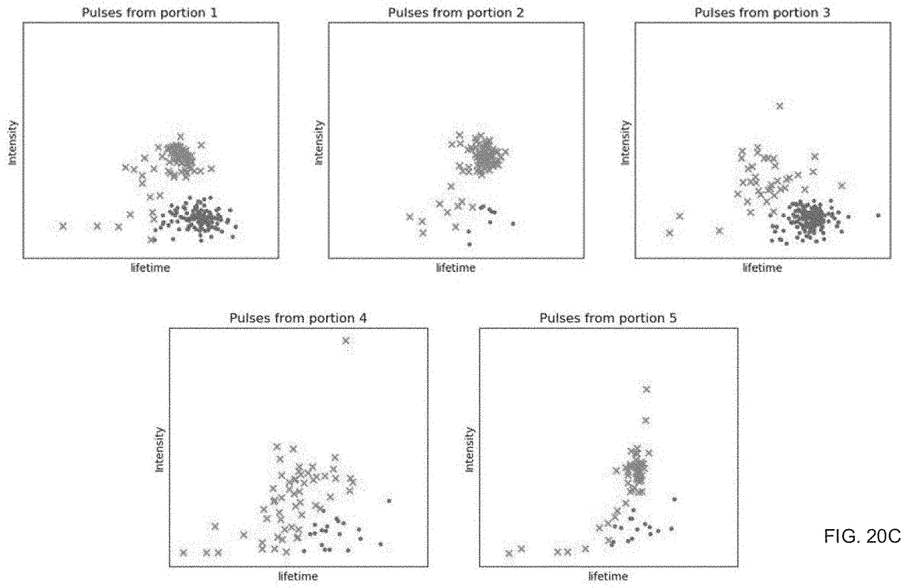

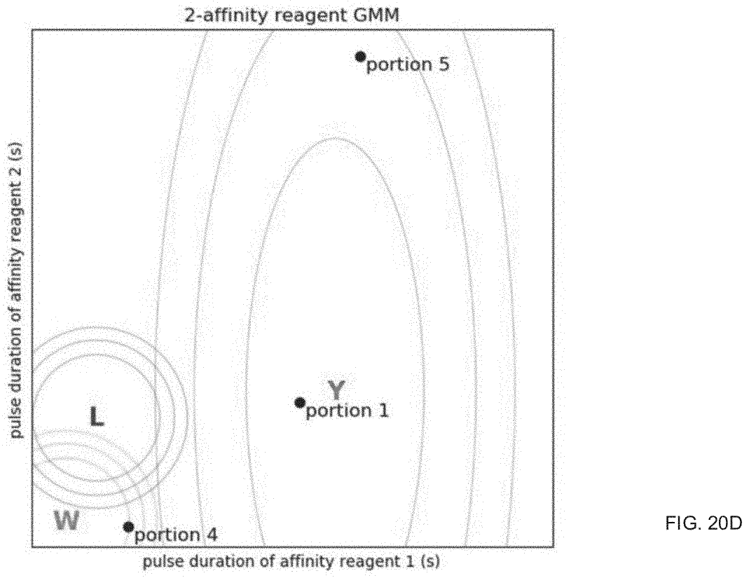

FIGS. 20A-20C depict an alternate two-step approach to identifying amino acids, according to some embodiments. In the example of FIGS. 20A-20C, a first clustering model may be developed to identify characteristic properties of data produced from affinity reagents, and to thereby allow for these reagents to be distinguished from one another. This technique may be beneficial if multiple affinity reagents are producing data at the same time in a signal trace. Subsequently, additional clustering models may be applied based on which portions of the data are determined to comprise data generated by the various affinity reagents.

As shown in FIG. 20A, a signal trace is analyzed and determined to include five portions that are labeled accordingly in the figure. In the case that at least some of these portions include data produced by more than one affinity reagent, a machine learning model trained on data from a single affinity reagent may not accurately categorize such portions of data. As such, initially a first clustering model is developed based on the data from all of the portions in the signal trace. This first clustering model is represented in FIG. 20B, which shows luminescence lifetime and pulse intensity for the pulses in all of the portions 1 through 5. The first clustering model may thereby identify characteristic properties of the affinity reagents—as shown in FIG. 20B, two different clusters are identified representing data from two different affinity reagents.

Subsequently, pulse lifetime and intensity data for pulses from each of the five portions of data shown in FIG. 20A may be arranged separately, as shown in FIG. 20C. In arranging this data, the clustering assignments of the pulses from the first clustering model are utilized. As may be noted, pulses from some portions—namely, portions 1, 3, 4 and 5—include data from both of the two clusters of the first clustering model. In contrast, portion 2 only primarily includes data from a single cluster.

By identifying which of the clusters are present in each portion utilizing the first clustering model, a different GMM model may be selected based on which clusters are present. For instance, data for portions 1, 3, 4 and 5 may be assigned an amino acid based on a GMM model trained specifically for properties of the affinity reagents corresponding to each cluster in the first clustering model. This result is shown in FIG. 20D, which plots the mean pulse duration for data points from the first cluster against the mean pulse duration for data points from the second cluster (the data point for portion 3 is not shown within the visible area shown in FIG. 20D). As such, each portion may be categorized appropriately. In contrast, portion 2 may instead be classified by separate GMM models that were trained on only the properties of their respective binders.

FIG. 11 illustrates an example structure of a convolutional neural network (CNN) 1100 for identifying amino acids, according to some embodiments of the technology described herein. In some embodiments, the CNN 1100 may be trained by performing process 600 described above with reference to FIG. 6A. In some embodiments, the trained CNN 1100 obtained from process 600 may be used to perform process 610 described above with reference to FIG. 6B.

In the example embodiment of FIG. 11, the CNN 1100 receives an input 1102A. In some embodiments, the input 1102A may be a collection of frames specifying numbers of photons in time intervals of time periods after light pulses. In some embodiments, the input 1102A may be arranged in a data structure such as data structure 910 described above with reference to FIG. 9B. In the example embodiment of FIG. 11, the input 1102A includes 1000 frames of data for two time intervals forming a 2×1000 input matrix. In some embodiments, the input 1102A may comprise a set of frames associated with a binding interaction of a reagent with an amino acid (e.g., as identified during process 700). In some embodiments, the input 1102A may be values of one or more properties of detected binding interactions (e.g., pulse duration, inter-pulse duration, wavelength, luminescence intensity, and/or luminescence lifetime), and/or values of one or more parameters derived from the properties.

In some embodiments, the CNN 1100 includes one or more convolutional layers 1102 in which the input 1102A is convolved with one or more filters. In the example embodiment of FIG. 11, the input 1102A is convolved with a first series of 16 2×50 filters in a first convolution layer. The convolution with 16 filters results in a 16×951 output 1102B. In some embodiments, the CNN 1100 may include a pooling layer after the first convolutional layer. As an example, the CNN 1100 may perform pooling by taking the maximum value in windows of the output of the first convolutional layer to obtain the output 1102B.

In the example embodiment of FIG. 11, the output 1102B of the first convolutional layer is then convolved with a second set of one or more filters in a second convolution layer. The output 1102B is convolved with a set of one or more 1×6 filters to obtain the output 1102C. In some embodiments, the CNN 1100 may include a pooling layer (e.g., a max pooling layer) after the second convolutional layer.

In the example embodiment of FIG. 11, the CNN 1100 includes a flattening step 1104 in which the output of the convolution 1102 is flattened to generate a flattened output 1106A. In some embodiments, the CNN 1100 may be configured to flatten the output 1102C by converting an 8×946 output matrix into a one dimensional vector. In the example embodiment of FIG. 11, the 8×43 output 1102C is converted into a 1×7568 vector 1106A. The vector 1106A may be inputted into a fully connected layer to generate a score for each possible class. In the example embodiment of FIG. 11, the possible classes are the twenty common amino acids, and blank (-). A softmax operation 1106 is then performed on the output of the fully connected layer to obtain the output 1110. In some embodiments, the softmax operation 1106 may convert the score for each of the classes into a respective probability. An argmax operation 1108 is then performed on the output 1110 to obtain a classification. The argmax operation 1108 may select the class having the highest probability in the output 1110. As an example, the output may identify an amino acid in a binding reaction with a reagent during a time period represented by the input 1102A. As another example, the output may identify that there was no binding interaction of a reagent with an amino acid during the time period by outputting a classification of blank (-).

FIG. 12 illustrates an example of a connectionist temporal classification (CTC)-fitted neural network model 1200 for identifying amino acids of a polypeptide, according to some embodiments of the technology described herein. In some embodiments, the CTC-fitted neural network model 1200 may be trained by performing process 600 described above with reference to FIG. 6A. In some embodiments, the trained CTC-fitted neural network model 1200 obtained from process 600 may be used to perform process 610 described above with reference to FIG. 6B.

In the example embodiment of FIG. 12, the model 1200 is configured to receive data collected by a protein sequencing device (e.g., protein sequencing device 502). As an example, the model 1200 may be a machine learning model used by the protein identification system 502C of protein sequencing device 502. The data may be accessed from detected light emissions by luminescent labels during interactions of reagents with amino acids. In some embodiments, the data may be arranged as multiple series of numbers of photons and/or frames as described above with reference to FIG. 9B. In some embodiments, portions of the data collected by the protein sequencing device 1220 may be provided as a series of inputs to the model 1200. As an example, the model 1200 may be configured to receive a first 2×400 input specifying numbers of photons detected in two time intervals after each of 400 light pulses.

In the example embodiment of FIG. 12, the model 1200 includes a feature extractor 1204. In some embodiments, the feature extractor may be an encoder of a trained autoencoder. The autoencoder may be trained, and the decoder from the autoencoder may be implemented as the feature extractor 1204. The encoder may be configured to encode the input as values of one or more features 1206.

In the example embodiment of FIG. 12, the feature values 1206 determined by the feature extractor 1204 are input into a predictor 1208 which outputs a probability matrix 1210 indicating a series of probability values for each possible class. In the example embodiment of FIG. 12, the classes include amino acids that reagents can bind with (e.g., twenty common amino acids, and blank (-)). As an example, the predictor 1208 may output a 21×50 matrix indicating a series of 50 probability values for each of the classes. The probability matrix 1210 may be used to generate an output 1230 identifying an amino acid sequence corresponding to data collected by protein sequencing device 1220. In some embodiments, the amino acid sequence may be determined from the probability matrix 1210. As an example, a beam search may be performed to obtain the output 1230 of an amino acid sequence. In some embodiments, the output may be matched to one of multiple sequences of amino acids specifying respective proteins (e.g., as performed at block 618 of process 610). As an example, the output may be used to generate a hidden Markov model (HMM) that is used to select an amino acid sequence, from a set of multiple amino acid sequences, that aligns most closely with the HMM of the multiple sequences of proteins.

In some embodiments, the feature extractor 1204 may be trained separately from the predictor 1208. As an example, the feature extractor 1204 may be obtained by training an autoencoder. The encoder from the autoencoder may then be used as the feature extractor 1204. In some embodiments, the predictor 1208 may be separately trained using the CTC loss function 1212. The CTC loss function 1212 may train the predictor 1208 to generate an output that can be used to generate the output 1230.

In some embodiments, multiple probability matrices may be combined. A second input may be accessed from data obtained by the protein sequencing device 1220. The second input may be a second portion of the data obtained by the protein sequencing device 1220. In some embodiments, the second input may be obtained by shifting by a number of points in the data obtained by the protein sequencing device 1220. As an example, the second input may be a second 400×2 input matrix obtained by shifting 8 points in the data obtained from the sequencer 420. A probability matrix corresponding to the second input may be obtained from the predictor 1208, and combined with a first probability matrix corresponding to a first input. As an example, the second probability matrix may be added to the first probability matrix. As another example, the second probability matrix may be shifted and added to the first probability matrix. The combined probability matrices may then be used to obtain the output 1230 identifying an amino acid sequence.

In some embodiments, the feature extractor 1204 may be a neural network. In some embodiments, the neural network may be a convolutional neural network (CNN). In some embodiments, the CNN may include one or more convolutional layers and one or more pooling layers. The CNN may include a first convolutional layer in which the input from the protein sequencing device 1220 is convolved with a set of filters. As an example, the input may be convolved with a set of 16 10×2 filters using a stride of 1×1 to generate a 16×400×2 output. An activation function may be applied to the output of the first convolutional layer. As an example, an ReLU activation function may be applied to the output of the first convolutional layer. In some embodiments, the CNN may include a first pooling layer after the first convolutional layer. In some embodiments, the CNN may apply a maxpool operation on the output of the first convolutional layer. As an example, a 2×2 filter with a 1×1 stride may be applied to a 16×400×2 output to obtain a 200×1 output.

In some embodiments, the CNN may include a second convolutional layer. The second convolutional layer may receive the output of the first pooling layer as an input. As an example, the second convolutional layer may receive the 200×1 output of the first pooling layer as input. The second convolutional layer may involve convolution with a second set of filters. As an example, in the second convolutional layer, the 200×1 input may be convolved with a second set of 16 10×1 filters with a stride of 1×1 to generate a 16×200 output. An activation function may be applied to the output of the second convolutional layer. As an example, an ReLU activation function may be applied to the output of the second convolutional layer. In some embodiments, the CNN may include a second pooling layer after the second convolutional layer. In some embodiments, the CNN may apply a maxpool operation on the output of the second convolution layer. As an example, a 4×1 filter with a 4×1 stride may be applied to the 16×200 output of the second convolutional layer to obtain a 16×50 output.

In some embodiments, the feature extractor 1204 may be a recurrent neural network (RNN). As an example, the feature extractor 1204 may be an RNN trained to encode data received from the protein sequencing device 1220 as values of one or more features. In some embodiments, the feature extractor 1204 may be a long short-term memory (LSTM) network. In some embodiments, the feature extractor 1204 may be a gated recurrent unit (GRU) network.

In some embodiments, the predictor 1208 may be a neural network. In some embodiments the neural network may be a GRU network. In some embodiments, the GRU network may be bidirectional. As an example, the GRU network may receive the 16×50 output of the feature extractor 1204 which is provided as input to the GRU network. As an example, the GRU network may have 64 hidden layers that generate a 50×128 output. In some embodiments, GRU network may use a tanh activation function. In some embodiments, predictor 1208 may include a fully connected layer. The output of the GRU network may be provided as input to the fully connected layer, which generates a 21×50 output matrix. The 21×50 matrix may include a series of values for each possible output class. In some embodiments, the predictor 1208 may be configured to apply a softmax function on the output of the fully connected layer to obtain the probability matrix 1210.

As discussed above in relation to FIG. 7, portions of a signal trace may be identified in order to identify values to be input into a trained machine learning model. Each portion, or region of interest (ROI), may be associated with a particular luminescent reagent in that characteristics of the signal produced in the ROI are indicative of the reagent. For example, in FIG. 3, three ROIs denoted K, F and Q are identified between cleavage events. Identifying these ROIs may therefore represent an initial step of selecting portions of data, as in the method of FIG. 7, prior to extracting features from each ROI for input to the trained machine learning model.

An illustrative approach for identifying ROIs is illustrated in FIGS. 14A-14C. For purposes of explanation, FIG. 14A depicts an illustrative signal trace that comprises a large number of pulses (measured light emissions) as described above. In general, such a signal trace may include a number of ROIs that each correspond to pulses produced by a particular affinity reagent. In the approach to be described further below, a wavelet transformation may be applied to some or all of the signal trace to generate a plurality of wavelet coefficients, which are depicted in FIG. 14B. These wavelet coefficients represent properties of the original signal trace, as may be noted by comparing the positions of the various features in FIG. 14B with corresponding changes in the pulses in FIG. 14A.

As shown in FIG. 14C, the wavelet coefficients may be analyzed to identify candidate ROIs. The dark vertical bars in FIG. 14C represent a measurement of the wavelet coefficients that indicates a beginning or an end of an ROI may be present at that position. In some cases, as discussed below, the candidate ROIs may be further analyzed to exclude some candidate ROIs based on a measure of confidence of how likely the candidate is to be a real ROI.

FIG. 15 is a flowchart of a method of identifying ROIs using the wavelet approach outlined above, according to some embodiments. Method 1500 may for instance be utilized in block 702 in method 700 of FIG. 7, in which portions (ROIs) of the data are identified prior to providing data to the machine learning model for each portion.

Method 1500 begins in act 1502 in which a wavelet decomposition is performed of some or all of a signal trace comprising pulses. In some embodiments, the wavelet decomposition may include a discrete wavelet transformation (DWT), which may be performed to any suitable level of decomposition. In some embodiments, act 1502 may comprise generating coefficients with a decomposition level of at least 10, or between 10 and 20, or between 15 and 20, or between 17 and 18. In some embodiments, the decomposition level may be selected dynamically based on one or more properties of the signal trace (e.g., frame duration, inter-pulse duration, etc.).

According to some embodiments, the wavelet decomposition performed in act 1502 may be performed using any suitable discrete wavelet and/or wavelet family, including but not limited to Haar, Daubechies, biorthogonal, coiflet, or symlet.

Since the wavelet transformation may produce a fewer number of coefficients than there are measurements (frames) in the signal trace, one or more operations may be performed in act 1502 to generate additional data values in between the generated wavelet coefficients so that there are the same number of values to be compared between the wavelet coefficients and the signal trace. For instance, data values may be generated by interpolation between the wavelet coefficients via any suitable interpolation method or methods. For example, data values may be generated via nearest-neighbor interpolation, via linear interpolation, via polynomial interpolation, via spline interpolation, or via combinations thereof.

Irrespective of how the wavelet coefficients are calculated in act 1502, and irrespective of whether or not additional data values are generated as described above, in act 1504 edges are detected based on the wavelet coefficients. In the subsequent description, act 1504 will be described as comprising operations performed based on the wavelet coefficients, although it will be appreciated that this description is applicable to both only a set of wavelet coefficients produced from the wavelet transformation in act 1502, and to a combination of wavelet coefficients combined with interpolated data values.

In some embodiments, edges may be detected by measuring the slope of the wavelet coefficients in act 1504. For instance, an average slope over one or more neighboring values within the coefficients may be calculated and an edge detected when the average slope is above a suitable threshold value. In some embodiments, the threshold value may be zero—that is, when the slope of the coefficients goes from zero to above zero, an edge may be detected, and when the slope of the coefficients is negative and rises to zero, an edge may also be detected. This may allow for leading and falling edges of an ROI to be detected.

In some embodiments, a magnitude of a detected edge may be calculated in act 1504. The magnitude may for instance be the size of the slope of the wavelet coefficients immediately adjacent to the detected edge. Thus, an edge that rises quickly may be identified as having a different magnitude from an edge that rises more slowly.

In act 1506, one or more candidate ROIs may be identified within the signal trace based on the edges detected in act 1504. In some embodiments, candidate ROIs may be identified as a region between starting and ending edges. For instance, in the example of FIG. 14C, the initial two edges identified may be considered to be the start and end of the first ROI, thereby allowing the region 1405 to be identified as a candidate ROI.

According to some embodiments, act 1506 may comprise a significance test to determine if a significant change in pulse duration of the pulses occurs within a candidate ROI. If a change in pulse duration is found to be significant by some measure, the candidate ROI may be split into two or more ROIs that each exhibit different pulse durations. For instance, a time position and/or pulse position within the candidate ROI may be identified as a point at which to split the ROI into two new ROIs (thus, the first new ROI may end at the split point and the second new ROI may begin at the split point). This process may be recursive in that an ROI may be split, then the new ROIs generated by splitting the initial ROI examined and split again, etc. It will also be appreciated that any pulse characteristic or characteristics may be examined to determine whether to split a candidate ROI, as this approach is not limited to use of only the pulse duration.

Irrespective of how the candidate ROIs are identified from the detected edges in act 1506, in act 1508 the candidate ROIs may optionally be scored and low-scoring ROIs excluded from consideration. Act 1508 may thereby allow for culling of spurious ROIs that are identified in act 1506 but that are unlikely to represent an actual ROI.

According to some embodiments, a value of a scoring function may be calculated for each ROI in act 1508. The scoring function may be a function of several variables, including but not limited to: the mean slope of the wavelet coefficients at the leading and/or trailing edges of the candidate ROI; the mean or median amplitude of the wavelet coefficients within the ROI; the pulse rate within the ROI; an estimate of the noise level within the entire signal trace; the pulse rate within the entire signal trace; or combinations thereof.

According to some embodiments, the scoring function may take the following form to calculate the confidence score for the i′th candidate ROI Cᵢ:

$$- {C}_{i} = \frac{{E}_{i} \times {M}_{i} \times P {r}_{i}}{Nt \times P R}$$

wherein Eᵢ is the mean of the slope of the wavelet coefficients at the leading and trailing edges of the candidate ROI, Mᵢ is the median amplitude of the wavelet coefficients within the ROI, Prᵢ is the pulse rate within the ROI, Nt is an estimate of the noise level within the entire signal trace (e.g., the full wavelet entropy of the signal trace), and PR is the pulse rate within the entire signal trace.

According to some embodiments, act 1508 may comprise excluding any ROIs that have a calculated score below a threshold value. For instance, in the case where the score is given by the equation above, candidate ROIs scoring below some threshold value may be excluded from subsequent consideration.

As discussed above in relation to FIG. 7, values for input to the machine learning model may include any parameters derived from a portion of data, including parameters that describe a distribution fit to pulse parameters. Moreover, during training of the machine learning model, data produced from known affinity reagents may be fit to a suitable distribution so that the machine learning model is trained to recognize affinity reagents based on the parameters of the distribution they exhibit.

FIGS. 16A-16B depict two illustrative approaches that may be applied in this manner, according to some embodiments. In the example of FIG. 16A, pulse durations for a portion of a signal trace corresponding to an affinity reagent associated with a known amino acid are fit to a power law distribution. The dark line 1601 represents the distribution of pulse durations exhibited by the relevant signal trace data and the light line 1602 represents a line described by the power law Cx^{a}, where C and a are constants and x is the pulse duration. By training the machine learning model in this manner, each affinity reagent may be associated with its own values (or own distributions of values) of C and a.

The approach illustrated by FIG. 16A and the subsequent discussion is based on the possibility that a single pulse duration value (or other pulse parameter) may not fully represent the types of measurements produced by a particular affinity reagent. Rather, each affinity reagent may naturally produce a range of pulse parameter values. But, the characteristics of the range may be different for each affinity reagent—hence, the distributions are characteristic of the reagents rather than a particular value.

FIG. 16B is an example of using a sum of exponential functions (also referred to as exponential states) to represent the data produced by a given affinity reagent. As shown in FIG. 16B, pulse durations for a portion of a signal trace corresponding to an affinity reagent associated with a known amino acid are fit to a sum of exponential functions. The dark line 1611 represents the distribution of pulse durations exhibited by the relevant signal trace data and the mid-grey line 1612 represents a line described by a sum of exponential functions. These exponential functions are illustrated as light grey lines 1615 and 1616. Mathematically, the sum of exponential functions may be given by:

Σbᵢe^{a}^{ᵢ}^{x}

where aᵢ and bᵢ are values for the i′th exponential function. In the case depicted in FIG. 16B, therefore, the values that may be fit to the data 1611 are a₁, a₂, b₁, and b₂.

FIGS. 17A-17B depict an approach in which pulse duration values are fit to a sum of three exponential functions, wherein each fitted distribution includes a common exponential function, according to some embodiments. In the example of FIGS. 17A-17B, a sum of three exponential functions is fit to the pulse duration distribution for each of two illustrative dipeptides FA and YA. The sum of exponential functions may be given as in the above equation, wherein the same values of a₀ and b₀ are used to fit each of the distributions, with the remaining values a₁, a₂, b₁, and b₂ being fit for each distribution separately. In particular, FIG. 17A depicts data 1701 being fit to a sum 1702 of exponential functions 1705, 1715 and 1716, with function 1705 being the common exponential function. FIG. 17B depicts data 1711 being fit to a sum 1712 of exponential functions 1705, 1718 and 1719.

The approach of FIGS. 17A-17B may have an advantage that the common state represented by the values a₀ and b₀ may represent a common component of the distributions that is present for all dipeptides. This common component may for instance represent noise inherent to the measurement device and/or noise inherent to use of affinity reagents to produce the signal traces.

According to some embodiments, training the machine learning model using this approach may comprise the following. First, model the dynamics of the system as a three-component system that is a function of pulse durations:

$${G}^{( n )} ( x ) = A \frac{{e}^{- x / α}}{α} + B \frac{{e}^{- x / {β}_{0}}}{{β}_{0}} + C \frac{{e}^{- x / {β}_{1}}}{{β}_{1}}$$

where the value of a is shared over all dipeptides, but the remaining parameters A, B, C, β₀ and β₁ are specific to a particular dipeptide referenced by the index n.

The function G (x) may be constrained to sum to unity over the range of pulse durations observed:

$${\int }_{{d}_{0}}^{{d}_{1}} {G}^{( n )} ( x ) d x = 1$$

where d₀ and d₁ are the lower and upper range of the possible pulse durations observed.

During training of the machine learning model, the parameters of G (x) may be determined by minimizing the negative log likelihood of the model. That is, minimizing:

− [symbol] ln(p⁽ⁿ⁾)

[symbol]

where p⁽ⁿ⁾ is the probability of observing the data given the model parameters:

p⁽ⁿ⁾=(X⁽ⁿ⁾;α,βₖ⁽ⁿ⁾)

with X⁽ⁿ⁾ being the set of pulse durations observed for the training data.

When performing protein identification, this model may be applied by calculating p⁽ⁿ⁾ over all n. The model prediction is then the dipeptide represented by the n with the largest values of Σ ln(p⁽ⁿ⁾).

It will be appreciated that the above-described example of modeling the distribution of pulse durations using a sum of exponential functions is provided as one example of describing the pulse characteristics of data produced by a particular affinity reagent and/or dipeptide. Other approaches may rely on multiple distributions of different pulse characteristics and may apply various machine learning techniques to train the machine learning model to identify proteins based on parameters from the multiple distributions.

In some embodiments, distributions may be based on probabilities of measuring a particular pulse characteristic or characteristics given a particular affinity reagent interacting with the protein to produce the observed pulses. In some embodiments, distributions may be based on probabilities of measuring a particular pulse characteristic or characteristics given a particular terminal dipeptide being present when the observed pulses were observed. The above two cases are not necessary identical, since a particular affinity reagent may produce a different distribution of pulse characteristics when interacting with one dipeptide versus another. Similarly, the same dipeptide may cause different pulse characteristics to be produced when interacting with one affinity reagent versus another.

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Further, though advantages of the present invention are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

For instance, techniques are described herein for sequencing biological polymers, such as peptides, polypeptides and/or proteins. It will be appreciated that the techniques described may be applied to any suitable polymer of amino acids, and that any references herein to sequencing, identifying an amino acid, etc., should not be viewed as being limiting with respect to the particular polymer. As such, any references to proteins, polypeptides, peptides, etc. herein are, unless indicated otherwise, provided as illustrative examples and it will be understood that such references may equally apply to other polymers of amino acids not expressly identified. Furthermore, any biological polymer may be sequenced using the techniques described herein, including but not limited to DNA and/or RNA.

Furthermore, as used herein, “sequencing,” “sequence determination,” “determining a sequence,” and like terms, in reference to a polypeptide or protein includes determination of partial sequence information as well as full sequence information of the polypeptide or protein. That is, the terminology includes sequence comparisons, fingerprinting, probabilistic fingerprinting, and like levels of information about a target molecule, as well as the express identification and ordering of each amino acid of the target molecule within a region of interest. In some embodiments, the terminology includes identifying a single amino acid of a polypeptide. In yet other embodiments, more than one amino acid of a polypeptide is identified. As used herein, in some embodiments, “identifying,” “determining the identity,” and like terms, in reference to an amino acid includes determination of an express identity of an amino acid as well as determination of a probability of an express identity of an amino acid. For example, in some embodiments, an amino acid is identified by determining a probability (e.g., from 0% to 100%) that the amino acid is of a specific type, or by determining a probability for each of a plurality of specific types. Accordingly, in some embodiments, the terms “amino acid sequence,” “polypeptide sequence,” and “protein sequence” as used herein may refer to the polypeptide or protein material itself and is not restricted to the specific sequence information (e.g., the succession of letters representing the order of amino acids from one terminus to another terminus) that biochemically characterizes a specific polypeptide or protein.

According to some aspects, a method is provided of training a machine learning model for identifying amino acids of polypeptides, the method comprising using at least one computer hardware processor to perform accessing training data obtained for binding interactions of one or more reagents with amino acids and training the machine learning model using the training data to obtain a trained machine learning model for identifying amino acids of polypeptides.

According to some embodiments, the machine learning model comprises a mixture model.

According to some embodiments, the mixture model comprises a Gaussian Mixture Model (GMM).

According to some embodiments, the machine learning model comprises a deep learning model.

According to some embodiments, the deep learning model comprises a convolutional neural network.

According to some embodiments, the deep learning model comprises a connectionist temporal classification (CTC)-fitted neural network.

According to some embodiments, training the machine learning model using the training data comprises applying a supervised training algorithm to the training data.

According to some embodiments, training the machine learning model using the training data comprises applying a semi-supervised training algorithm to the training data.

According to some embodiments, training the machine learning model using the training data comprises applying an unsupervised training algorithm to the training data.

According to some embodiments, the machine learning model comprises a clustering model and training the machine learning model comprises identifying a plurality of clusters of the clustering model, each of the plurality of clusters associated with one or more amino acids.

According to some embodiments, the data for binding interactions of one or more reagents with amino acids comprises one or more parameters describing a distribution of at least one property of signal pulses detected for a binding interaction.

According to some embodiments, the data for binding interactions of one or more reagents with amino acids comprises one or more parameters derived from at least one property of signal pulses detected for a binding interaction.

According to some embodiments, the data for binding interactions of one or more reagents with amino acids comprises pulse duration values, each pulse duration value indicating a duration of a signal pulse detected for a binding interaction.

According to some embodiments, the data obtained for binding interactions of one or more reagents with amino acids comprises inter-pulse duration values, each inter-pulse duration value indicating a duration of time between consecutive signal pulses detected for a binding interaction.

According to some embodiments, the data obtained for binding interactions of one or more reagents with amino acids comprises one or more pulse duration values, and one or more inter-pulse duration values.

According to some embodiments, the method further comprises training the machine learning model to output, for each of a plurality of locations in a polypeptide, one or more likelihoods that one or more respective amino acids is present at the location.

According to some embodiments, training the machine learning model comprises identifying a plurality of portions of the data, each portion corresponding to a respective one of the binding interactions, providing each one of the plurality of portions as input to the machine learning model to obtain an output corresponding to the each one portion of data, and training the machine learning model using outputs corresponding to the plurality of portions.

According to some embodiments, the output corresponding to the portion of data indicates one or more likelihoods that one or more respective amino acids is present at a respective one of a plurality of locations.

According to some embodiments, identifying the plurality of portions of the data comprises identifying one or more points in the data corresponding to cleavage of one or more of the amino acids, and identifying the plurality of portions of the data based on the identified one or more points corresponding to the cleavage of the one or more amino acids.

According to some embodiments, identifying the plurality of portions of the data comprises determining, from the data, a value of a summary statistic for at least one property of the binding interactions, identifying one or more points in the data at which a value of the at least one property deviates from the value of the summary statistic by a threshold amount, and identifying the plurality of portions of the data based on the identified one or more points.

According to some embodiments, the data for binding interactions of one or more reagents with amino acids comprises data obtained from detected light emissions by one or more luminescent labels.

According to some embodiments, the data obtained from detected light emissions by the one or more luminescent labels comprises luminescence lifetime values.

According to some embodiments, the data obtained from detected light emissions by the one or more luminescent labels comprises luminescence intensity values.

According to some embodiments, the data obtained from detected light emissions by the one or more luminescent labels comprises wavelength values, each wavelength value indicating a wavelength of light emitted during a binding interaction.

According to some embodiments, the light emissions are responsive to a series of light pulses, and the data includes, for each of at least some of the light pulses, a respective number of photons detected in each of a plurality of time intervals which are part of a time period after the light pulse.

According to some embodiments, training the machine learning model comprises providing the data as input to the machine learning model by arranging the data into a data structure having columns wherein a first column holds a respective number of photons in each of a first and second time interval which are part of a first time period after a first light pulse in the series of light pulses, and a second column holds a respective number of photons in each of a first and second time interval which are part of a second time period after a second light pulse in the series of light pulses.

According to some embodiments, training the machine learning model comprises providing the data as input to the machine learning model by arranging the data into a data structure having rows wherein each of the rows holds numbers of photons in a respective time interval corresponding to the at least some light pulses.

According to some embodiments, providing the data as input to the machine learning model comprises arranging the data in an image, wherein a first pixel of the image specifies a first number of photons detected in a first time interval of a first time period after a first pulse of the at least some pulses.

According to some embodiments, a second pixel of the image specifies a second number of photons detected in a second time interval of the first time period after a the first pulse of the at least some pulses.

According to some embodiments, a second pixel of the image specifies a second number of photons in a first time interval of a second time period after a second pulse of the at least some pulses.

According to some embodiments, providing the data as input to the trained machine learning model comprises arranging the data in an image, wherein each pixel of the image specifies a number of photons detected in a respective time interval of a time period after a pulse of the at least some pulses.

According to some embodiments, the one or more luminescent labels are associated with at least one of the one or more reagents.

According to some embodiments, the luminescent labels are associated with at least some of the amino acids.

According to some embodiments, the training data represents binding interactions of the one or more reagents with amino acids of a single molecule.

According to some embodiments, the training data represents binding interactions of the one or more reagents with amino acids of a plurality of molecules.

According to some aspects, a system is provided for training a machine learning model for identifying amino acids of polypeptides, the system comprising at least one processor, and at least one non-transitory computer-readable storage medium storing instructions that, when executed by the at least one processor, cause the at least one processor to perform accessing training data obtained for binding interactions of one or more reagents with amino acids, and training the machine learning model using the training data to obtain a trained machine learning model for identifying amino acids of polypeptides.

According to some embodiments, the machine learning model comprises a mixture model.

According to some embodiments, the mixture model comprises a Gaussian Mixture Model (GMM).

According to some embodiments, the machine learning model comprises a deep learning model.

According to some embodiments, the deep learning model comprises a convolutional neural network.

According to some embodiments, the deep learning model comprises a connectionist temporal classification (CTC)-fitted neural network.

According to some embodiments, training the machine learning model using the training data comprises applying a supervised training algorithm to the training data.

According to some embodiments, training the machine learning model using the training data comprises applying a semi-supervised training algorithm to the training data.

According to some embodiments, training the machine learning model using the training data comprises applying an unsupervised training algorithm to the training data.

According to some embodiments, the machine learning model comprises a clustering model and training the machine learning model comprises identifying a plurality of clusters of the clustering model, each of the plurality of clusters associated with one or more amino acids.

According to some embodiments, the data for binding interactions of one or more reagents with amino acids comprises pulse duration values, each pulse duration value indicating a duration of a signal pulse detected for a binding interaction.

According to some embodiments, the data for binding interactions of one or more reagents with amino acids comprises one or more parameters describing a distribution of at least one property of signal pulses detected for a binding interaction.

According to some embodiments, the data for binding interactions of one or more reagents with amino acids comprises one or more parameters derived from at least one property of signal pulses detected for a binding interaction.

According to some embodiments, the data obtained for binding interactions of one or more reagents with amino acids comprises inter-pulse duration values, each inter-pulse duration value indicating a duration of time between consecutive signal pulses detected for a binding interaction.

According to some embodiments, the data obtained for binding interactions of one or more reagents with amino acids comprises one or more pulse duration values, and one or more inter-pulse duration values.

According to some embodiments, the instructions, when executed by the at least one processor, further cause the at least one processor to perform training the machine learning model to output, for each of a plurality of locations in a polypeptide, one or more likelihoods that one or more respective amino acids is present at the location.

According to some embodiments, training the machine learning model comprises identifying a plurality of portions of the data, each portion corresponding to a respective one of the binding interactions, providing each one of the plurality of portions as input to the machine learning model to obtain an output corresponding to the each one portion of data, and training the machine learning model using outputs corresponding to the plurality of portions.

According to some embodiments, the output corresponding to the portion of data indicates one or more likelihoods that one or more respective amino acids is present at a respective one of a plurality of locations.

According to some embodiments, identifying the plurality of portions of the data comprises identifying one or more points in the data corresponding to cleavage of one or more of the amino acids, and identifying the plurality of portions of the data based on the identified one or more points corresponding to the cleavage of the one or more amino acids.

According to some embodiments, identifying the plurality of portions of the data comprises determining, from the data, a value of a summary statistic for at least one property of the binding interactions, identifying one or more points in the data at which a value of the at least one property deviates from the value of the summary statistic by a threshold amount, and identifying the plurality of portions of the data based on the identified one or more points.

According to some embodiments, the data for binding interactions of one or more reagents with amino acids comprises data obtained from detected light emissions by one or more luminescent labels.

According to some embodiments, the data obtained from detected light emissions by the one or more luminescent labels comprises luminescence lifetime values.

According to some embodiments, the data obtained from detected light emissions by the one or more luminescent labels comprises luminescence intensity values.

According to some embodiments, the data obtained from detected light emissions by the one or more luminescent labels comprises wavelength values, each wavelength value indicating a wavelength of light emitted during a binding interaction.

According to some embodiments, the light emissions are responsive to a series of light pulses, and the data includes, for each of at least some of the light pulses, a respective number of photons detected in each of a plurality of time intervals which are part of a time period after the light pulse.

According to some embodiments, training the machine learning model comprises providing the data as input to the machine learning model by arranging the data into a data structure having columns wherein a first column holds a respective number of photons in each of a first and second time interval which are part of a first time period after a first light pulse in the series of light pulses, and a second column holds a respective number of photons in each of a first and second time interval which are part of a second time period after a second light pulse in the series of light pulses.

According to some embodiments, training the machine learning model comprises providing the data as input to the machine learning model by arranging the data into a data structure having rows wherein each of the rows holds numbers of photons in a respective time interval corresponding to the at least some light pulses.

According to some embodiments, providing the data as input to the machine learning model comprises arranging the data in an image, wherein a first pixel of the image specifies a first number of photons detected in a first time interval of a first time period after a first pulse of the at least some pulses.

According to some embodiments, a second pixel of the image specifies a second number of photons detected in a second time interval of the first time period after a the first pulse of the at least some pulses.

According to some embodiments, a second pixel of the image specifies a second number of photons in a first time interval of a second time period after a second pulse of the at least some pulses.

According to some embodiments, providing the data as input to the trained machine learning model comprises arranging the data in an image, wherein each pixel of the image specifies a number of photons detected in a respective time interval of a time period after a pulse of the at least some pulses.

According to some embodiments, the one or more luminescent labels are associated with at least one of the one or more reagents.

According to some embodiments, the luminescent labels are associated with at least some of the amino acids.

According to some embodiments, the training data represents binding interactions of the one or more reagents with amino acids of a single molecule.

According to some embodiments, the training data represents binding interactions of the one or more reagents with amino acids of a plurality of molecules.

According to some aspects, at least one non-transitory computer-readable storage medium is provided storing instructions that, when executed by at least one processor, cause the at least one processor to perform accessing training data obtained for binding interactions of one or more reagents with amino acids, and training a machine learning model using the training data to obtain a trained machine learning model for identifying amino acids of polypeptides.

According to some embodiments, the machine learning model comprises a mixture model.

According to some embodiments, the mixture model comprises a Gaussian Mixture Model (GMM).

According to some embodiments, the machine learning model comprises a deep learning model.

According to some embodiments, the deep learning model comprises a convolutional neural network.

According to some embodiments, the deep learning model comprises a connectionist temporal classification (CTC)-fitted neural network.

According to some embodiments, training the machine learning model using the training data comprises applying a supervised training algorithm to the training data.

According to some embodiments, training the machine learning model using the training data comprises applying a semi-supervised training algorithm to the training data.

According to some embodiments, training the machine learning model using the training data comprises applying an unsupervised training algorithm to the training data.

According to some embodiments, the machine learning model comprises a clustering model and training the machine learning model comprises identifying a plurality of clusters of the clustering model, each of the plurality of clusters associated with one or more amino acids.

According to some embodiments, the data for binding interactions of one or more reagents with amino acids comprises one or more parameters describing a distribution of at least one property of signal pulses detected for a binding interaction.

According to some embodiments, the data for binding interactions of one or more reagents with amino acids comprises one or more parameters derived from at least one property of signal pulses detected for a binding interaction.

According to some embodiments, the data for binding interactions of one or more reagents with amino acids comprises pulse duration values, each pulse duration value indicating a duration of a signal pulse detected for a binding interaction.

According to some embodiments, the data obtained for binding interactions of one or more reagents with amino acids comprises inter-pulse duration values, each inter-pulse duration value indicating a duration of time between consecutive signal pulses detected for a binding interaction.

According to some embodiments, the data obtained for binding interactions of one or more reagents with amino acids comprises one or more pulse duration values, and one or more inter-pulse duration values.

According to some embodiments, the instructions, when executed by at least one processor, further cause the at least one processor to perform training the machine learning model to output, for each of a plurality of locations in a polypeptide, one or more likelihoods that one or more respective amino acids is present at the location.

According to some embodiments, training the machine learning model comprises identifying a plurality of portions of the data, each portion corresponding to a respective one of the binding interactions, providing each one of the plurality of portions as input to the machine learning model to obtain an output corresponding to the each one portion of data, and training the machine learning model using outputs corresponding to the plurality of portions.

According to some embodiments, the output corresponding to the portion of data indicates one or more likelihoods that one or more respective amino acids is present at a respective one of a plurality of locations.

According to some embodiments, identifying the plurality of portions of the data comprises identifying one or more points in the data corresponding to cleavage of one or more of the amino acids, and identifying the plurality of portions of the data based on the identified one or more points corresponding to the cleavage of the one or more amino acids.

According to some embodiments, identifying the plurality of portions of the data comprises determining, from the data, a value of a summary statistic for at least one property of the binding interactions, identifying one or more points in the data at which a value of the at least one property deviates from the value of the summary statistic by a threshold amount, and identifying the plurality of portions of the data based on the identified one or more points.

According to some embodiments, the data for binding interactions of one or more reagents with amino acids comprises data obtained from detected light emissions by one or more luminescent labels.

According to some embodiments, the data obtained from detected light emissions by the one or more luminescent labels comprises luminescence lifetime values.

According to some embodiments, the data obtained from detected light emissions by the one or more luminescent labels comprises luminescence intensity values.

According to some embodiments, the data obtained from detected light emissions by the one or more luminescent labels comprises wavelength values, each wavelength value indicating a wavelength of light emitted during a binding interaction.

According to some embodiments, the light emissions are responsive to a series of light pulses, and the data includes, for each of at least some of the light pulses, a respective number of photons detected in each of a plurality of time intervals which are part of a time period after the light pulse.

According to some embodiments, training the machine learning model comprises providing the data as input to the machine learning model by arranging the data into a data structure having columns wherein a first column holds a respective number of photons in each of a first and second time interval which are part of a first time period after a first light pulse in the series of light pulses, and a second column holds a respective number of photons in each of a first and second time interval which are part of a second time period after a second light pulse in the series of light pulses.

According to some embodiments, training the machine learning model comprises providing the data as input to the machine learning model by arranging the data into a data structure having rows wherein each of the rows holds numbers of photons in a respective time interval corresponding to the at least some light pulses.

According to some embodiments, providing the data as input to the machine learning model comprises arranging the data in an image, wherein a first pixel of the image specifies a first number of photons detected in a first time interval of a first time period after a first pulse of the at least some pulses.

According to some embodiments, a second pixel of the image specifies a second number of photons detected in a second time interval of the first time period after a the first pulse of the at least some pulses.

According to some embodiments, a second pixel of the image specifies a second number of photons in a first time interval of a second time period after a second pulse of the at least some pulses.

According to some embodiments, providing the data as input to the trained machine learning model comprises arranging the data in an image, wherein each pixel of the image specifies a number of photons detected in a respective time interval of a time period after a pulse of the at least some pulses.

According to some embodiments, the one or more luminescent labels are associated with at least one of the one or more reagents.

According to some embodiments, the luminescent labels are associated with at least some of the amino acids.

According to some embodiments, the training data represents binding interactions of the one or more reagents with amino acids of a single molecule.

According to some embodiments, the training data represents binding interactions of the one or more reagents with amino acids of a plurality of molecules.

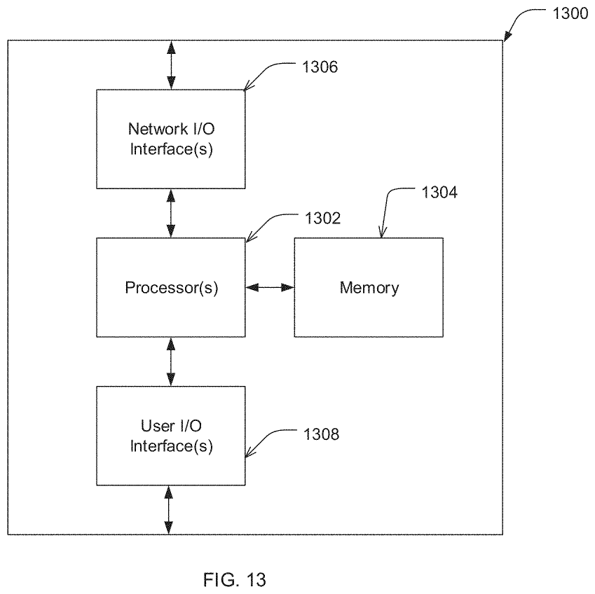

In some embodiments, systems and techniques described herein may be implemented using one or more computing devices. Embodiments are not, however, limited to operating with any particular type of computing device. By way of further illustration, FIG. 13 is a block diagram of an illustrative computing device 1300. Computing device 1300 may include one or more processors 1302 and one or more tangible, non-transitory computer-readable storage media (e.g., memory 1304). Memory 1304 may store, in a tangible non-transitory computer-recordable medium, computer program instructions that, when executed, implement any of the above-described functionality. Processor(s) 1302 may be coupled to memory 1304 and may execute such computer program instructions to cause the functionality to be realized and performed.

Computing device 1300 may also include a network input/output (I/O) interface 1306 via which the computing device may communicate with other computing devices (e.g., over a network), and may also include one or more user I/O interfaces 1308, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. As an example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. As an example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different from illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

What is claimed is:

1. A method for identifying a polypeptide, the method comprising:
using at least one computer hardware processor to perform:
accessing data for binding interactions of one or more reagents with amino acids of the polypeptide;
providing the data as input to a trained machine learning model to obtain output indicating, for each of a plurality of locations in the polypeptide, one or more likelihoods that one or more respective amino acids is present at the location; and
identifying the polypeptide based on the output obtained from the trained machine learning model.

2. The method of claim 1, wherein the one or more likelihoods that the one or more respective amino acids is present at the location include:
a first likelihood that a first amino acid is present at the location; and
a second likelihood that a second amino acid is present at the location.

3. The method of claim 1, wherein identifying the polypeptide comprises matching the obtained output to one of a plurality of amino acid sequences associated with respective proteins.

4. The method of claim 3, wherein matching the obtained output to the one of the plurality of amino acid sequences specifying respective proteins comprises:
generating a hidden Markov model (HMM) based on the obtained output; and
matching the HMM to the one of the plurality of amino acid sequences.

5. The method of claim 1, wherein the machine learning model comprises a Gaussian Mixture Model (GMM).

6. The method of claim 1, wherein the machine learning model comprises a clustering model comprising multiple clusters, each of the clusters being associated with one or more amino acids.

7. The method of claim 1, wherein the machine learning model comprises a deep learning model.

8. The method of claim 1, wherein the machine learning model comprises a convolutional neural network.

9. The method of claim 7, wherein the deep learning model comprises a connectionist temporal classification (CTC)-fitted neural network.

10. The method of claim 1, wherein the trained machine learning model is generated by applying a supervised training algorithm to training data.

11. The method of claim 1, wherein the trained machine learning model is generated by a applying a semi-supervised training algorithm to training data.

12. The method of claim 1, wherein the trained machine learning model is generated by applying an unsupervised training algorithm.

13. The method of claim 1, wherein the trained machine learning model is configured to output, for each of at least some of the plurality of locations in the polypeptide:
a probability distribution indicating, for each of multiple amino acids, a probability that the amino acid is present at the location.

14. The method of claim 1, wherein the data for binding interactions of one or more reagents with amino acids of the polypeptide comprises pulse duration values, each pulse duration value indicating a duration of a signal pulse detected for a binding interaction.

15. The method of claim 1, wherein the data for binding interactions of one or more reagents with amino acids of the polypeptide comprises inter-pulse duration values, each inter-pulse duration value indicating a duration of time between consecutive signal pulses detected for a binding interaction.

16. The method of claim 1, wherein the data for binding interactions of one or more reagents with amino acids of the polypeptide comprises one or more pulse duration values, and one or more inter-pulse duration values.

17. The method of claim 1, wherein providing the data as input to the trained machine learning model further comprises:
identifying a plurality of portions of the data, each portion corresponding to a respective one of the binding interactions; and
providing each one of the plurality of portions as input to the trained machine learning model to obtain an output corresponding to the each one portion of data.

18. The method of claim 17, wherein the output corresponding to the portion of data indicates one or more likelihoods that one or more respective amino acids is present at a respective one of the plurality of locations.

19. The method of claim 17, wherein identifying the plurality of portions of the data comprises:
identifying one or more points in the data corresponding to cleavage of one or more of the amino acids; and
identifying the plurality of portions of the data based on the identified one or more points corresponding to the cleavage of the one or more amino acids.

20. The method of claim 17, wherein identifying the plurality of portions of the data comprises generating a discrete wavelet transformation of the data.

21. The method of claim 17, wherein identifying the plurality of portions of the data comprises:
determining, from the data, a value of a summary statistic for at least one property of the binding interactions;
identifying one or more points in the data at which a value of the at least one property deviates from the value of the statistic by a threshold amount; and
identifying the plurality of portions of the data based on the identified one or more points.

22. The method of claim 1, wherein the data for binding interactions of one or more reagents with amino acids of the polypeptide comprises data obtained from detected light emissions by one or more luminescent labels.

23. The method of claim 22, wherein the data obtained from detected light emissions by the one or more luminescent labels comprises wavelength values, each wavelength value indicating a wavelength of light emitted during a binding interaction.

24. The method of claim 22, wherein the data obtained from detected light emissions by the one or more luminescent labels comprises luminescence lifetime values.

25. The method of claim 22, wherein the data detected light emissions by the one or more luminescent labels comprises luminescence intensity values.

26. The method of claim 22, wherein the light emissions are responsive to a series of light pulses, and the data includes, for each of at least some of the light pulses, a respective number of photons detected in each of a plurality of time intervals which are part of a time period after the light pulse.

27. The method of claim 26, wherein providing the data as input to the trained machine learning model comprises arranging the data into a data structure having columns, wherein:
a first column holds a respective number of photons in each of a first and second time interval which are part of a first time period after a first light pulse in the series of light pulses; and
a second column holds a respective number of photons in each of a first and second time interval which are part of a second time period after a second light pulse in the series of light pulses.

28. The method of claim 22, wherein the one or more luminescent labels are associated with at least one of the one or more reagents.

29. The method of claim 22, wherein the one or more luminescent labels are associated with at least some of the amino acids of the polypeptide.

30. The method of claim 1, wherein the plurality of locations include at least one relative location within the polypeptide.

31. A system for identifying a polypeptide, the system comprising:
at least one processor; and
at least one non-transitory computer-readable storage medium storing instructions that, when executed by the at least one processor, cause the at least one processor to perform a method comprising:
accessing data for binding interactions of one or more reagents with amino acids of the polypeptide;
providing the data as input to a trained machine learning model to obtain output indicating, for each of a plurality of locations in the polypeptide, one or more likelihoods that one or more respective amino acids is present at the location; and
identifying the polypeptide based on the output obtained from the trained machine learning model.

32. At least one non-transitory computer-readable storage medium storing instructions that, when executed by at least one processor, cause the at least one processor to perform a method, the method comprising:
accessing data for binding interactions of one or more reagents with amino acids of a polypeptide;
providing the data as input to a trained machine learning model to obtain output indicating, for each of a plurality of locations in the polypeptide, one or more likelihoods that one or more respective amino acids is present at the location; and
identifying the polypeptide based on the output obtained from the trained machine learning model.
